(19) United States
(12) Patent Application Publication
(10) Pub. No.: US 2024/0004293 A1
(43) Pub. Date: Jan. 4, 2024

(54) ACTINIC RAY-SENSITIVE OR RADIATION-SENSITIVE RESIN COMPOSITION, ACTINIC RAY-SENSITIVE OR RADIATION-SENSITIVE FILM, PATTERN FORMING METHOD, AND METHOD FOR MANUFACTURING ELECTRONIC DEVICE

(71) Applicant: FUJIFILM Corporation, Tokyo (JP)

(72) Inventors: Naoya HATAKEYAMA, Haibara-gun (JP); Akiyoshi GOTO, Haibara-gun (JP); Hideyuki ISHIHARA, Haibara-gun (JP); Michihiro SHIRAKAWA, Haibara-gun (JP); Yosuke BEKKI, Haibara-gun (JP)

(73) Assignee: FUJIFILM Corporation, Tokyo (JP)

(21) Appl. No.: 18/468,245

(22) Filed: Sep. 15, 2023

Related U.S. Application Data
(63) Continuation of application No. PCT/JP2022/010426, filed on Mar. 9, 2022.

(30) Foreign Application Priority Data
Mar. 22, 2021 (JP) 2021-047995
Jul. 30, 2021 (JP) 2021-126332

(51) Int. Cl. G03F 7/039; G03F 7/004; G03F 7/038; C08F 220/18; C08F 220/38; C08F 220/28; C08F 220/36; C08F 212/14

(52) U.S. Cl. CPC G03F 7/039; C08F 220/282; G03F 7/038; C08F 220/1809; C08F 220/382; C08F 220/281; C08F 220/1808; C08F 220/283; C08F 220/365; C08F 220/1807; C08F 220/1806; C08F 220/1811; C08F 212/22; C08F 220/1818; G03F 7/0045

(57) ABSTRACT

An actinic ray-sensitive or radiation-sensitive resin composition including: (A) a resin which is decomposed by action of acid to increase polarity; and (B) a compound which generates an acid by irradiation with an actinic ray or a radiation, in which the resin (A) and the acid generated from the compound (B) form a bond by the actinic ray or the radiation or by the action of acid.

### CROSS REFERENCE TO RELATED APPLICATION

This is a continuation of International Application No. PCT/JP2022/010426 filed on Mar. 9, 2022, and claims priority from Japanese Patent Application No. 2021-047995 filed on Mar. 22, 2021, and Japanese Patent Application No. 2021-126332 filed on Jul. 30, 2021, the entire disclosure of which are incorporated herein by reference.

### BACKGROUND OF THE INVENTION

### 1. Field of the Invention

The present invention relates to an actinic ray-sensitive or radiation-sensitive resin composition, an actinic ray-sensitive or radiation-sensitive film, a pattern forming method, and a method for manufacturing an electronic device.

### 2. Description of the Related Art

In processes for manufacturing semiconductor devices such as an integrated circuit (IC) and a large scale integrated circuit (LSI), nanofabrication by lithography using a photosensitive composition has been performed.

Examples of the lithographic method include a method in which a resist film is formed with the photosensitive composition, and then the obtained film is exposed and then developed. In particular, in recent years, studies have been conducted on the use of electron beam (EB) and extreme ultraviolet (EUV) light in addition to an ArF excimer laser during exposure, and an actinic ray-sensitive or radiation-sensitive resin composition suitable for EUV exposure has been developed.

In a formation of a resist pattern using EUV (wavelength: 13.5 nm) for the purpose of forming a fine pattern or using electron beam, requirements for various performances are stricter than a case of using ArF (wavelength: 193 nm) light or the like in the related art.

For example, JP2019-14704A discloses a resist composition that contains a resin which has an acid generator containing a salt represented by a specific structure and a resin and has an acid unstable group.

### SUMMARY OF THE INVENTION

In recent years, miniaturization of a pattern formed by using EUV light or electron beam has been promoted, and further improvement is required in a resolution performance or the like of the pattern. However, a resolution recently demanded, for example, a line width or space width of 20 nm or less, is an extremely high degree of fineness, and it is very difficult to achieve this resolution with the resist composition in the related art.

Accordingly, an object of the present invention is to provide an actinic ray-sensitive or radiation-sensitive resin composition in which, in a formation of an extremely fine pattern (for example, line-and-space pattern having a line width or space width of 20 nm or less, hole pattern of a pore diameter of 20 nm or less, and the like), a resolution is extremely excellent.

In addition, another object of the present invention is to provide an actinic ray-sensitive or radiation-sensitive film formed of the actinic ray-sensitive or radiation-sensitive resin composition, a pattern forming method, and a method for manufacturing an electronic device.

The present inventors have found that the above-described objects can be achieved by the following configurations.

[1]

An actinic ray-sensitive or radiation-sensitive resin composition comprising:
(A) a resin which is decomposed by action of acid to increase polarity; and
(B) a compound which generates an acid by irradiation with an actinic ray or a radiation, in which the resin (A) and the acid generated from the compound (B) form a bond by the actinic ray or the radiation or by the action of acid.

[2]

The actinic ray-sensitive or radiation-sensitive resin composition according to [1],
in which the resin (A) is a resin having a reactive moiety (1),
the compound (B) is an ionic compound having a reactive moiety (2) in an anionic moiety, and
a reactive species generated from one of the reactive moiety (1) or the reactive moiety (2) by the actinic ray or the radiation or by the action of acid reacts with the other of the reactive moiety (1) and the reactive moiety (2) to form the bond.

[3]

The actinic ray-sensitive or radiation-sensitive resin composition according to [2],
in which the compound (B) is an ionic compound having, as the reactive moiety (2) in the anionic moiety, a partial structure represented by any of General Formula (1), (2), or (3).

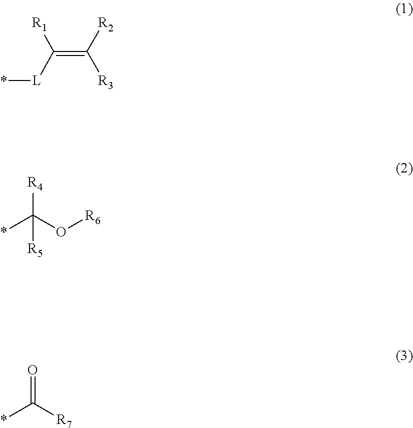

In the General Formula (1), R₁ to R₃ each independently represent a hydrogen atom or a substituent, L represents a single bond or a divalent linking group, and * represents a bonding position.

In the General Formula (2), R₄ to R₆ each independently represent a hydrogen atom or a substituent, and * represents a bonding position.

In the General Formula (3), R₇ represents a hydrogen atom or a substituent, and * represents a bonding position.

[4]

The actinic ray-sensitive or radiation-sensitive resin composition according to [3], wherein the partial structure is the partial structure represented by the General Formula (1) or the General Formula (3).

The actinic ray-sensitive or radiation-sensitive resin composition according to [3], in which the partial structure is a partial structure selected from the following structures.

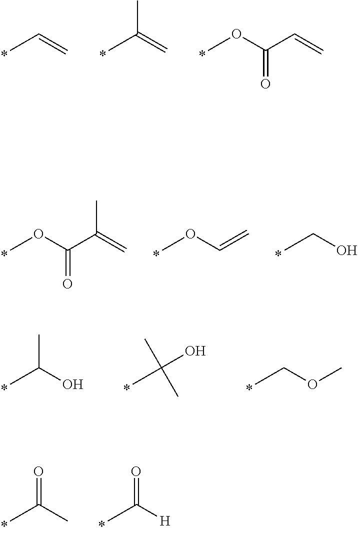

* represents a bonding position.

[6]

The actinic ray-sensitive or radiation-sensitive resin composition according to [5], in which the partial structure is a partial structure selected from the following structures.

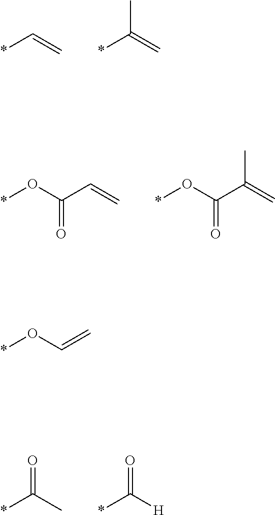

* represents a bonding position.

[7]

An actinic ray-sensitive or radiation-sensitive resin composition comprising:
(A) a resin which is decomposed by action of acid to increase polarity; and
(B) a compound which generates an acid by irradiation with an actinic ray or a radiation, in which the resin (A) is a resin having an acid group, an alcoholic hydroxyl group, or an acid-decomposable group, and
the compound (B) is an ionic compound having a partial structure represented by any of General Formula (1), (2), or (3) in an anionic moiety.

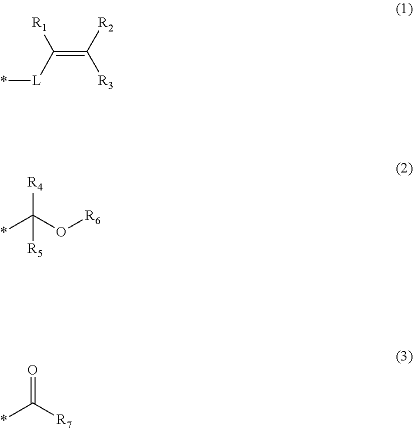

In the General Formula (1), R₁ to R₃ each independently represent a hydrogen atom or a substituent, L represents a single bond or a divalent linking group, and * represents a bonding position.

In the General Formula (2), R₄ to R₆ each independently represent a hydrogen atom or a substituent, and * represents a bonding position.

In the General Formula (3), R₇ represents a hydrogen atom or a substituent, and * represents a bonding position.

[8]

The actinic ray-sensitive or radiation-sensitive resin composition according to [7],
in which the partial structure is the partial structure represented by the General Formula (1) or the General Formula (3).

[8]

The actinic ray-sensitive or radiation-sensitive resin composition according to [7],
in which the partial structure is a partial structure selected from the following structures.

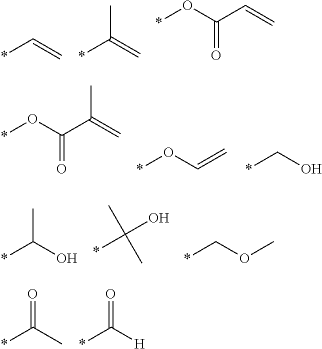

* represents a bonding position.

[10]

The actinic ray-sensitive or radiation-sensitive resin composition according to [9],
in which the partial structure is a partial structure selected from the following structures.

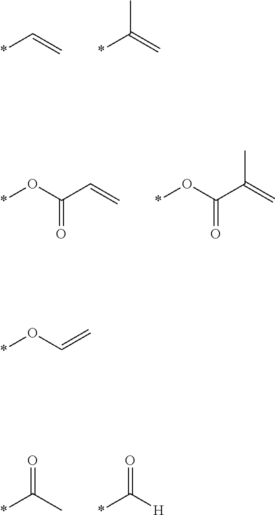

* represents a bonding position.

[11]

The actinic ray-sensitive or radiation-sensitive resin composition according to any one of [2] to [10],
in which the compound (B) has no partial structure represented by any of General Formula (11), (12), (13), or (14) in the anionic moiety.

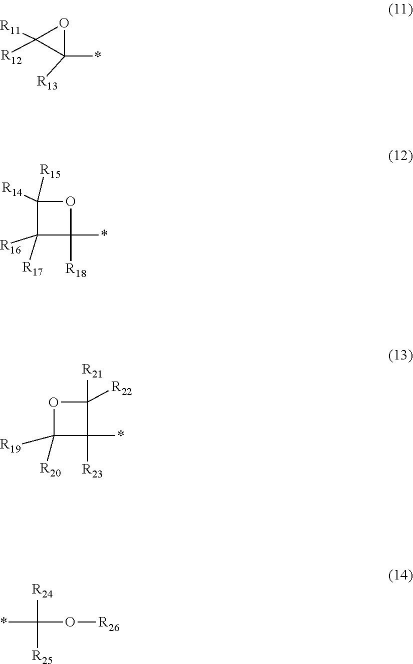

In the General Formula (11), R₁ to R₁₃ each independently represent a hydrogen atom or a substituent, and * represents a bonding position.

In the General Formula (12), R₁₄ to R₁₅ each independently represent a hydrogen atom or a substituent, and * represents a bonding position.

In the General Formula (13), R₁₉ to R₂₃ each independently represent a hydrogen atom or a substituent, and * represents a bonding position.

In the General Formula (14), R₂₄ to R₂₆ each independently represent a hydrogen atom or a substituent, and * represents a bonding position.

[12]

The actinic ray-sensitive or radiation-sensitive resin composition according to any one of [1] to [11],
in which the resin (A) has a dissociable hydrogen atom.

[13]

The actinic ray-sensitive or radiation-sensitive resin composition according to any one of [1] to [12],
in which the resin (A) has a phenolic hydroxyl group.

[14]

The actinic ray-sensitive or radiation-sensitive resin composition according to any one of [1] to [13],
in which the acid generated from the compound (B) includes an aromatic ring.

[15]

An actinic ray-sensitive or radiation-sensitive film formed of the actinic ray-sensitive or radiation-sensitive resin composition according to any one of [1] to [14].

[16]

A pattern forming method comprising:
forming an actinic ray-sensitive or radiation-sensitive film on a substrate using the actinic ray-sensitive or radiation-sensitive resin composition according to any one of [1] to [14];
exposing the actinic ray-sensitive or radiation-sensitive film; and
developing the exposed actinic ray-sensitive or radiation-sensitive film with a developer to form a pattern.

[17]

A method for manufacturing an electronic device, comprising:
the pattern forming method according to [16].

According to the present invention, it is possible to provide an actinic ray-sensitive or radiation-sensitive resin composition in which, in a formation of an extremely fine pattern (for example, line-and-space pattern having a line width or space width of 20 nm or less, hole pattern of a pore diameter of 20 nm or less, and the like), a resolution is extremely excellent.

In addition, according to the present invention, it is possible to provide an actinic ray-sensitive or radiation-sensitive film formed of the actinic ray-sensitive or radiation-sensitive resin composition, a pattern forming method, and a method for manufacturing an electronic device.

### DESCRIPTION OF THE PREFERRED EMBODIMENTS

Hereinafter, the present invention will be described in detail.

Description of configuration requirements described below may be made on the basis of representative embodiments of the present invention in some cases, but the present invention is not limited to such embodiments.

In notations for a group (atomic group) in the present specification, in a case where the group is cited without specifying that it is substituted or unsubstituted, the group includes both a group having no substituent and a group having a substituent as long as it does not impair the spirit of the present invention. For example, an “alkyl group” includes not only an alkyl group having no substituent (unsubstituted alkyl group), but also an alkyl group having a substituent (substituted alkyl group). In addition, an “organic group” in the present specification refers to a group including at least one carbon atom.

A substituent is preferably a monovalent substituent unless otherwise specified.

“Actinic ray” or “radiation” in the present specification means, for example, a bright line spectrum of a mercury lamp, far ultraviolet rays typified by an excimer laser, extreme ultraviolet rays (EUV light), X-rays, or electron beams (EB).

“Light” in the present specification means actinic ray or radiation.

Unless otherwise specified, “exposure” in the present specification encompasses not only exposure by a bright line spectrum of a mercury lamp, far ultraviolet rays typified by an excimer laser, extreme ultraviolet rays (EUV rights), X-rays, or the like, but also drawing by corpuscular beams such as electron beams and ion beams.

In the present specification, a numerical range expressed using “to” is used in a meaning of a range that includes the preceding and succeeding numerical values of “to” as the lower limit value and the upper limit value, respectively.

A bonding direction of divalent groups cited in the present specification is not limited unless otherwise specified. For example, in a case where Y in a compound represented by Formula “X—Y—Z” is —CO—O—, Y may be —CO—O— or —O—CO—. In addition, the above-described compound may be “X—CO—O—Z” or “X—O—CO—Z”.

In the present specification, (meth)acrylate represents acrylate and methacrylate, and (meth)acryl represents acryl and methacryl.

In the present specification, a weight-average molecular weight (Mw), a number-average molecular weight (Mn), and a dispersity (hereinafter, also referred to as “molecular weight distribution”) (Mw/Mn) are defined as values expressed in terms of polystyrene by means of gel permeation chromatography (GPC) measurement (solvent: tetrahydrofuran, flow amount (amount of a sample injected): 10 μL, columns: TSK gel Multipore HXL-M manufactured by Tosoh Corporation, column temperature: 40° C., flow rate: 1.0 mL/min, and detector: differential refractive index detector) using a GPC apparatus (HLC-8120GPC manufactured by Tosoh Corporation).

In the present specification, an acid dissociation constant (pKa) represents a pKa in an aqueous solution, and is specifically a value determined by computation from a value based on a Hammett's substituent constant and database of publicly known literature values, using the following software package 1.

Software Package 1: Advanced Chemistry Development (ACD/Labs) Software V 8.14 for Solaris (1994-2007 ACD/Labs).

In addition, the pKa can also be determined by a molecular orbital computation method. Examples of a specific method therefor include a method for performing calculation by computing H⁺ dissociation free energy in an aqueous solution based on a thermodynamic cycle. With regard to a computation method for H⁺ dissociation free energy, the H⁺ dissociation free energy can be computed by, for example, density functional theory (DFT), but various other methods have been reported in literature and the like, and are not limited thereto. There are a plurality of software applications capable of performing DFT, and examples thereof include Gaussian 16.

As described above, the pKa in the present specification refers to a value determined by computation from a value based on a Hammett's substituent constant and database of publicly known literature values, using the software package 1, but in a case where the pKa cannot be calculated by the method, a value obtained by Gaussian 16 based on density functional theory (DFT) shall be adopted.

In addition, the pKa in the present specification refers to a “pKa in an aqueous solution” as described above, but in a case where the pKa in an aqueous solution cannot be calculated, a “pKa in a dimethyl sulfoxide (DMSO) solution” shall be adopted.

“Solid content” is intended to be components which form an actinic ray-sensitive or radiation-sensitive film, and does not include a solvent. In addition, even in a case where a component is liquid, the component is included in the solid content as long as the component forms the actinic ray-sensitive or radiation-sensitive film.

In addition, in the present specification, the types of substituents, the positions of substituents, and the number of substituents in a case where it is described that “a substituent may be contained” are not particularly limited. The number of substituents may be, for example, one, two, three, or more. Examples of the substituent include a monovalent non-metal atomic group from which a hydrogen atom has been excluded, and the substituent can be selected from the following substituent T, for example.

(Substituent T)

Examples of the substituent T include halogen atoms such as a fluorine atom, a chlorine atom, a bromine atom, and an iodine atom; alkoxy groups such as a methoxy group, an ethoxy group, and a tert-butoxy group; aryloxy groups such as a phenoxy group and a p-tolyloxy group; alkoxycarbonyl groups such as a methoxycarbonyl group, a butoxycarbonyl group, and a phenoxycarbonyl group; acyloxy groups such as an acetoxy group, a propionyloxy group, and a benzoyloxy group; acyl groups such as an acetyl group, a benzoyl group, an isobutyryl group, an acryloyl group, a methacryloyl group, and a methoxalyl group; alkylsulfanyl groups such as a methylsulfanyl group and a tert-butylsulfanyl group; arylsulfanyl groups such as a phenylsulfanyl group and a p-tolylsulfanyl group; an alkyl group; an alkenyl group; a cycloalkyl group; an aryl group; a heteroaryl group; a hydroxyl group; a carboxy group; a formyl group; a sulfo group; a cyano group; an alkylaminocarbonyl group; an arylaminocarbonyl group; a sulfonamide group; a silyl group; an amino group; a monoalkylamino group; a dialkylamino group; an arylamino group; and a combination thereof.

[Actinic Ray-Sensitive or Radiation-Sensitive Resin Composition]

The actinic ray-sensitive or radiation-sensitive resin composition according to the embodiment of the present invention is an actinic ray-sensitive or radiation-sensitive resin composition containing (A) a resin which is decomposed by action of acid to increase polarity and (B) a compound which generates an acid by irradiation with an actinic ray or a radiation, in which the resin (A) and the acid generated from the compound (B) form a bond by the actinic ray or the radiation or by the action of acid.

In addition, the actinic ray-sensitive or radiation-sensitive resin composition according to the embodiment of the present invention is an actinic ray-sensitive or radiation-sensitive resin composition containing (A) a resin which is decomposed by action of acid to increase polarity and (B) a compound which generates an acid by irradiation with an actinic ray or a radiation, in which the resin (A) is a resin having an acid group, an alcoholic hydroxyl group, or an acid-decomposable group, and the compound (B) is an ionic compound having a partial structure represented by any of General Formula (1), (2), or (3) in an anionic moiety.

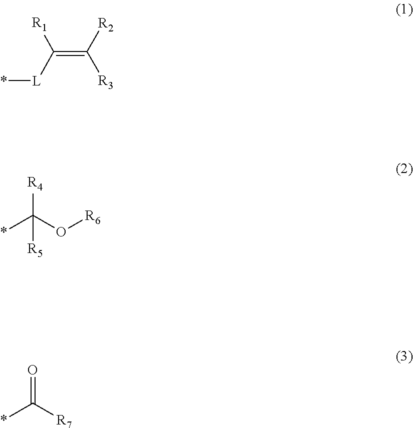

In General Formula (1), R₁ to R₃ each independently represent a hydrogen atom or a substituent, L represents a single bond or a divalent linking group, and * represents a bonding position.

In General Formula (2), R₄ to R₆ each independently represent a hydrogen atom or a substituent, and * represents a bonding position.

In General Formula (3), R₇ represents a hydrogen atom or a substituent, and * represents a bonding position.

A mechanism by which the objects of the present invention can be achieved through such configurations is not always clear, but is considered to be as follows by the present inventors.

As described above, the composition according to the embodiment of the present invention contains the resin (A), and the compound (B) as a photoacid generator, and in an exposed portion, the resin (A) and the acid generated from the compound (B) form a bond by actinic ray or radiation or by action of acid.

In addition, as described above, the composition according to the embodiment of the present invention contains the resin (A), and the compound (B) as a photoacid generator, the resin (A) is a resin having an acid group, an alcoholic hydroxyl group, or an acid-decomposable group, and the compound (B) is an ionic compound having a partial structure represented by any of General Formula (1), (2), or (3) in an anionic moiety. With such a configuration, as described above, in the exposed portion, it is considered that the acid generated from the compound (B) reacts with the resin (A) by actinic ray or radiation or by action of acid, and forms a bond with the resin (A).

As a result, in particular in the formation of the above-described extremely fine pattern, more precise reaction control is required in each region, and in the resist film according to the present invention, it is considered that unintended movement of the acid generated from the compound (B) in the exposed portion after exposure is suppressed with high accuracy. Therefore, it is considered that the desired reaction easily proceeds only in the desired region, and the extremely fine pattern can be obtained.

Accordingly, in the formation of the extremely fine pattern (for example, line-and-space pattern having a line width or space width of 20 nm or less, hole pattern of a pore diameter of 20 nm or less, and the like), it is considered that a pattern with extremely excellent resolution is formed.

[Components of Actinic Ray-Sensitive or Radiation-Sensitive Resin Composition]

Hereinafter, components which can be contained in the actinic ray-sensitive or radiation-sensitive resin composition (hereinafter, also referred to as a composition according to the embodiment of the present invention) will be described in detail.

The actinic ray-sensitive or radiation-sensitive resin composition according to the embodiment of the present invention is typically a resist composition, and may be a positive tone resist composition or a negative tone resist composition. In addition, the resist composition may be either a resist composition for alkali development or a resist composition for organic solvent development. The composition according to the embodiment of the present invention is typically a chemically amplified resist composition.

As described above, the above-described resin (A) and the acid generated from the above-described compound (B) form a bond by actinic ray or radiation or by action of acid.

The acid in “by actinic ray or radiation or by action of acid” is not particularly limited as long as it con form the above-described bond, but is typically the acid generated from the above-described compound (B).

In addition, examples of the above-described bond include a covalent bond. The above-described bond is formed in the exposed portion.

It is preferable that the above-described resin (A) is a resin having a reactive moiety (1), the above-described compound (B) is an ionic compound having a reactive moiety (2) in an anionic moiety, and a reactive species generated from one of the reactive moiety (1) or the reactive moiety (2) by the actinic ray or the radiation or by the action of acid reacts with the other of the reactive moiety (1) and the reactive moiety (2) to form the above-described bond.

The above-described reactive moiety (1) is not particularly limited, and examples thereof include an acid group, an alcoholic hydroxyl group, and an acid-decomposable group.

Details of Each Group Will be Described Later.

The above-described reactive moiety (2) is not particularly limited, and examples thereof include a partial structure represented by any of General Formula (1), (2), or (3).

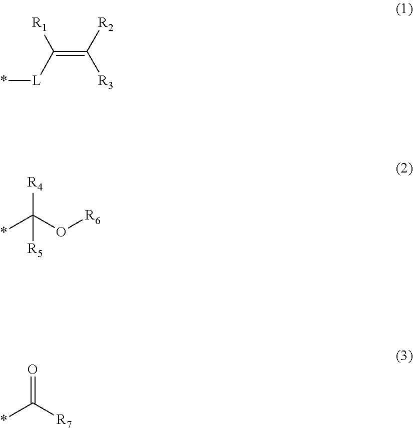

In General Formula (1), R₁ to R₃ each independently represent a hydrogen atom or a substituent, L represents a single bond or a divalent linking group, and * represents a bonding position.

In General Formula (2), R₄ to R₆ each independently represent a hydrogen atom or a substituent, and * represents a bonding position.

In General Formula (3), R₇ represents a hydrogen atom or a substituent, and * represents a bonding position.

Details of Each Group Will be Described Later.

It is preferable that the above-described compound (B) has the above-described partial structure as the above-described reactive moiety (2) in the anionic moiety.

The reactive species generated by actinic ray or radiation or by action of acid typically originates from one of the above-described reactive moiety (1) or the above-described reactive moiety (2).

The above-described reactive species is not particularly limited as long as it can react with the other of the above-described reactive moiety (1) or the above-described reactive moiety (2) to form a bond.

The reactive species generated from the above-described reactive moiety (2) is not particularly limited, and examples thereof include a moiety which is to be a carbocation in a case where the acid generated from the compound (B) attacks another compound (B) at the above-described partial structure represented by any of General Formula (1), (2), or (3).

In addition, examples of the reactive species also include a carbon atom charged with δ⁺, which is not to be the carbocation.

Examples of an aspect in which the reactive species generated from the above-described reactive moiety (2) reacts with the above-described reactive moiety (1) to form a bond include the following.

As described above, in a case where the acid generated from the compound (B) is involved in the reaction, the above-described carbocation or the above-described carbon atom charged with δ⁺ is subjected to nucleophilic attack by the acid group, alcoholic hydroxyl group, or acid-decomposable group as the reactive moiety (1) of the resin (A), and a bond between the above-described resin (A) and the above-described compound (B) is formed.

The reactive species generated from the above-described reactive moiety (1) is not particularly limited, and examples thereof include, in a case of using a resin including a phenolic hydroxyl group as the acid group, a radical of an oxygen atom in which a hydrogen atom is eliminated from the hydroxyl group by actinic ray or radiation, and a radical generated by elimination of a hydrogen atom on an aromatic ring which is at an ortho position to the hydroxyl group.

Examples of an aspect in which the reactive species generated from the above-described reactive moiety (1) reacts with the above-described partial structure represented by any of General Formula (1), (2), or (3) as the above-described reactive moiety (2) to form a bond include the following.

As described above, in a case where actinic ray or radiation is involved in the reaction, the radical moiety in the above-described resin (A) attacks the above-described partial structure represented by any of General Formula (1), (2), or (3) as the above-described reactive moiety (2) of the compound (B), and a bond between the above-described resin (A) and the above-described compound (B) is formed.

The above-described reactive moieties (1) and the above-described reactive moieties (2) may be one or a plurality of each.

<(A) Resin>

The actinic ray-sensitive or radiation-sensitive resin composition (hereinafter, also referred to as “composition”) contains a resin (A) which is decomposed by action of acid to increase polarity (hereinafter, also referred to as “resin (A)”).

The resin (A) is typically an acid-decomposable resin, and usually includes a repeating unit having a group having a polarity that increases through decomposition by action of acid (hereinafter, also referred to as “acid-decomposable group”), preferably includes a repeating unit having an acid-decomposable group.

Therefore, in the pattern forming method according to the embodiment of the present invention, typically, in a case where an alkali developer is adopted as a developer, a positive tone pattern is suitably formed, and in a case where an organic developer is adopted as a developer, a negative tone pattern is suitably formed.

As the repeating unit having an acid-decomposable group, in addition to (repeating Unit having an acid-decomposable group) described below, (repeating unit having an acid-decomposable group including an unsaturated bond) is preferable.

As a preferred aspect, the above-described resin (A) has a reactive moiety (1).

The above-described reactive moiety (1) is not particularly limited, and examples thereof include an acid group, an alcoholic hydroxyl group, and an acid-decomposable group.

The acid group is not particularly limited, but an acid group having a pKa of 13 or less is preferable. An acid dissociation constant of the above-described acid group is preferably 13 or less, more preferably 3 to 13, and still more preferably 5 to 10.

As the acid group, for example, a carboxyl group, a phenolic hydroxyl group, a fluoroalcohol group, a sulfonic acid group, or a sulfonamide group is preferable, and a phenolic hydroxyl group or a fluoroalcohol group is more preferable. The phenolic hydroxyl group represents a hydroxyl group directly bonded to an aromatic ring. The fluoroalcohol group is preferably a hexafluoroisopropanol group.

In addition, in the above-described hexafluoroisopropanol group, one or more (preferably one or two) fluorine atoms may be substituted with a group (an alkoxycarbonyl group and the like) other than a fluorine atom. —C(CF₃)(OH)—CF₂— formed as above is also preferable as the acid group. In addition, one or more fluorine atoms may be substituted with a group other than a fluorine atom to form a ring including —C(CF₃)(OH)—CF₂—.

The alcoholic hydroxyl group is a hydroxyl group bonded to a hydrocarbon group and refers to a hydroxyl group other than a hydroxyl group (phenolic hydroxyl group) directly bonded to an aromatic ring, and an aliphatic alcohol group (for example, a hexafluoroisopropanol group and the like) having an α-position substituted with an electron withdrawing group such as a fluorine atom is excluded as the hydroxyl group. The alcoholic hydroxyl group is preferably a hydroxyl group having an acid dissociation constant (pKa) from 12 to 20.

The acid-decomposable group is a group which is decomposed by action of acid to form a polar group. The acid-decomposable group preferably has a structure in which a polar group is protected by a leaving group which is eliminated by action of acid. That is, the resin (A) has a repeating unit having a group which is decomposed by action of acid to generate a polar group. The resin having this repeating unit has an increased polarity by action of acid, an increased solubility in an alkali developer, and a decreased solubility in an organic solvent.

The polar group is preferably an alkali-soluble group, examples thereof include an acidic group (typically, a group which dissociates in a 2.38%-by-mass tetramethylammonium hydroxide aqueous solution), such as a carboxyl group, a phenolic hydroxyl group, a fluorinated alcohol group, a sulfonic acid group, a phosphoric acid group, a sulfonamide group, a sulfonylimide group, an (alkylsulfonyl)(alkylcarbonyl)methylene group, an (alkylsulfonyl)(alkylcarbonyl)imide group, a bis(alkylcarbonyl)methylene group, a bis(alkylcarbonyl)imide group, a bis(alkylsulfonyl)methylene group, a bis(alkylsulfonyl)imide group, a tris(alkylcarbonyl)methylene group, and a tris(alkylsulfonyl)methylene group; and an alcoholic hydroxyl group.

Among those, as the polar group, a carboxyl group, a phenolic hydroxyl group, a fluorinated alcohol group (preferably, a hexafluoroisopropanol group), or a sulfonic acid group is preferable.

Examples of the leaving group which is eliminated by action of acid include groups represented by Formulae (Y1) to (Y4).

—C(Rx₁)(Rx₂)(Rx₃) Formula (Y1):

—C(═O)OC(Rx₁)(Rx₂)(Rx₃) Formula (Y2):

—C(R₃₆)(R₃₇)(OR₃₈) Formula (Y3):

—C(Rn)(H)(Ar) Formula (Y4):

In Formula (Y1) and Formula (Y2), Rx₁ to Rx₃ each independently represent a (linear or branched) alkyl group, a (monocyclic or polycyclic) cycloalkyl group, an (linear or branched) alkenyl group, or a (monocyclic or polycyclic) aryl group. In a case where all of Rx₁ to Rx₃ are (linear or branched) alkyl groups, it is preferable that at least two of Rx₁ to Rx₃ are methyl groups.

Among these, it is preferable that Rx₁ to Rx₃ each independently represent a linear or branched alkyl group, and it is more preferable that Rx₁ to Rx₃ each independently represent a linear alkyl group.

Two of Rx₁ to Rx₃ may be bonded to each other to form a monocycle or a polycycle.

As the alkyl group of Rx₁ to Rx₃, an alkyl group having 1 to 5 carbon atoms, such as a methyl group, an ethyl group, an n-propyl group, an isopropyl group, an n-butyl group, an isobutyl group, and a t-butyl group, is preferable.

As the cycloalkyl group of Rx₁ to Rx₃, a monocyclic cycloalkyl group such as a cyclopentyl group and a cyclohexyl group, or a polycyclic cycloalkyl group such as a norbornyl group, a tetracyclodecanyl group, a tetracyclododecanyl group, and an adamantyl group is preferable.

As the aryl group of Rx₁ to Rx₃, an aryl group having 6 to 10 carbon atoms is preferable, and examples thereof include a phenyl group, a naphthyl group, and an anthryl group.

As the alkenyl group of Rx₁ to Rx₃, a vinyl group is preferable.

A cycloalkyl group is preferable as the ring formed by the bonding of two of Rx₁ to Rx₃. As a cycloalkyl group formed by the bonding of two of Rx₁ to Rx₃, a monocyclic cycloalkyl group such as a cyclopentyl group and a cyclohexyl group, or a polycyclic cycloalkyl group such as a norbornyl group, a tetracyclodecanyl group, a tetracyclododecanyl group, and an adamantyl group is preferable, and a monocyclic cycloalkyl group having 5 or 6 carbon atoms is more preferable.

In the cycloalkyl group formed by the bonding of two of Rx₁ to Rx₃, one of methylene groups constituting the ring may be replaced with a heteroatom such as an oxygen atom, with a group including a heteroatom, such as a carbonyl group, or with a vinylidene group. In addition, in the cycloalkyl group, one or more of the ethylene groups constituting the cycloalkane ring may be replaced with a vinylene group.

With regard to the group represented by Formula (Y1) or Formula (Y2), for example, an aspect in which Rx₁ is a methyl group or an ethyl group and Rx₂ and Rx₃ are bonded to each other to form the above-described cycloalkyl group is preferable.

For example, in a case where the composition according to the embodiment of the present invention is an actinic ray-sensitive or radiation-sensitive resin composition for EUV exposure, it is also preferable that the alkyl group, cycloalkyl group, alkenyl group, and aryl group represented by Rx₁ to Rx₃ and the ring formed by bonding two of Rx₁ to Rx₃ further have a fluorine atom or an iodine atom as a substituent.

In Formula (Y3), R₃₆ to R₃₈ each independently represent a hydrogen atom or a monovalent organic group. R₃₇ and R₃₈ may be bonded to each other to form a ring. Examples of the monovalent organic group include an alkyl group, a cycloalkyl group, an aryl group, an aralkyl group, and an alkenyl group. It is also preferable that R₃₆ is a hydrogen atom.

The alkyl group, the cycloalkyl group, the aryl group, and the aralkyl group may include a heteroatom such as an oxygen atom, and/or a group including a heteroatom, such as a carbonyl group. For example, in the above-described alkyl group, cycloalkyl group, aryl group, and aralkyl group, one or more of methylene groups may be replaced with a heteroatom such as an oxygen atom and/or with a group including a heteroatom, such as a carbonyl group.

In addition, R₃₈ and another substituent included in the main chain of the repeating unit may be bonded to each other to form a ring. A group formed by the mutual bonding of R₃₈ and another substituent on the main chain of the repeating unit is preferably an alkylene group such as a methylene group.

For example, in a case where the composition according to the embodiment of the present invention is an actinic ray-sensitive or radiation-sensitive resin composition for EUV exposure, it is also preferable that the monovalent organic group represented by R₃₆ to R₃₈ and the ring formed by bonding R₃₇ and R₃₈ with each other further have a fluorine atom or an iodine atom as a substituent.

As Formula (Y3), a group represented by Formula (Y3-1) is preferable.

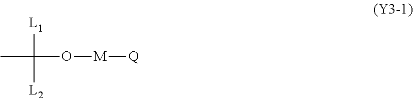

Here, L₁ and L₂ each independently represent a hydrogen atom, an alkyl group, a cycloalkyl group, an aryl group, or a group formed by a combination thereof (for example, a group formed by a combination of an alkyl group and an aryl group).

M represents a single bond or a divalent linking group.

Q represents an alkyl group which may include a heteroatom, a cycloalkyl group which may include a heteroatom, an aryl group which may include a heteroatom, an amino group, an ammonium group, a mercapto group, a cyano group, an aldehyde group, or a group formed by a combination thereof (for example, a group formed by a combination of an alkyl group and a cycloalkyl group).

In the alkyl group and the cycloalkyl group, for example, one of methylene groups may be replaced with a heteroatom such as an oxygen atom or with a group including a heteroatom, such as a carbonyl group.

It is preferable that one of L₁ or L₂ is a hydrogen atom, and the other is an alkyl group, a cycloalkyl group, an aryl group, or a group formed by a combination of an alkylene group and an aryl group.

At least two of Q, M, or L₁ may be bonded to each other to form a ring (preferably a 5- or 6-membered ring).

From the viewpoint of pattern miniaturization, L₂ is preferably a secondary or tertiary alkyl group, and more preferably a tertiary alkyl group. Examples of the secondary alkyl group include an isopropyl group, a cyclohexyl group, and a norbornyl group, and examples of the tertiary alkyl group include a tert-butyl group and an adamantane group. In these aspects, since a glass transition temperature (Tg) and an activation energy are increased, it is possible to suppress fogging in addition to ensuring a film hardness.

For example, in a case where the composition according to the embodiment of the present invention is an actinic ray-sensitive or radiation-sensitive resin composition for EUV exposure, it is also preferable that the alkyl group, cycloalkyl group, aryl group, and combination of these groups represented by L₁ and L₂ further have a fluorine atom or an iodine atom as a substituent. In addition, it is also preferable that the above-described alkyl group, cycloalkyl group, aryl group, and aralkyl group include a heteroatom such as an oxygen atom in addition to the fluorine atom and the iodine atom (that is, in the above-described alkyl group, cycloalkyl group, aryl group, and aralkyl group, for example, one of methylene groups may be replaced with a heteroatom such as an oxygen atom or with a group including a heteroatom, such as a carbonyl group).

In addition, for example, in a case where the composition according to the embodiment of the present invention is an actinic ray-sensitive or radiation-sensitive resin composition for EUV exposure, in the alkyl group which may include a heteroatom, the cycloalkyl group which may include a heteroatom, the aryl group which may include a heteroatom, the amino group, the ammonium group, the mercapto group, the cyano group, the aldehyde group, and the group formed by a combination thereof, which are represented by Q, the heteroatom is also preferably a heteroatom selected from the group consisting of a fluorine atom, an iodine atom, and an oxygen atom.

In Formula (Y4), Ar represents an aromatic ring group. Rn represents an alkyl group, a cycloalkyl group, or an aryl group. Rn and Ar may be bonded to each other to form a non-aromatic ring. Ar is preferably an aryl group.

For example, in a case where the composition according to the embodiment of the present invention is a composition for EUV exposure, it is also preferable that the aromatic ring group represented by Ar and the alkyl group, cycloalkyl group, and aryl group represented by Rn further have a fluorine atom or an iodine atom as a substituent.

From the viewpoint that the acid decomposability of the repeating unit is excellent, in a case where a non-aromatic ring is directly bonded to a polar group (or a residue thereof) in a leaving group which protects the polar group, it is also preferable that a ring member atom adjacent to the ring member atom directly bonded to the polar group (or a residue thereof) in the non-aromatic ring has no halogen atom such as a fluorine atom as a substituent.

In addition, the leaving group which is eliminated by action of acid may be a 2-cyclopentenyl group having a substituent (an alkyl group and the like), such as a 3-methyl-2-cyclopentenyl group, and a cyclohexyl group having a substituent (an alkyl group and the like), such as a 1,1,4,4-tetramethylcyclohexyl group.

As described above, the acid-decomposable group can be the reactive moiety (1), and specifically, it is as follows.

In the exposed portion, the leaving group is eliminated from the acid-decomposable group by acid, and a polar group is generated. Such a polar group can serve as the reactive moiety (1).

The above-described acid is typically the acid generated from the compound (B).

The reactive species generated from the above-described reactive moiety (1) is not particularly limited, and examples thereof include, in a case of using a resin including a phenolic hydroxyl group as the acid group, a radical of an oxygen atom in which a hydrogen atom is eliminated from the hydroxyl group by actinic ray or radiation, and a radical generated by elimination of a hydrogen atom on an aromatic ring which is at an ortho position to the hydroxyl group.

As a preferred aspect, the above-described resin (A) has an acid group, an alcoholic hydroxyl group, or an acid-decomposable group.

Each of the acid group, the alcoholic hydroxyl group, or the acid-decomposable group is as described above.

Each of the acid group, the alcoholic hydroxyl group, or the acid-decomposable group can be the reactive moiety (1). The reactive moiety (1) may be a moiety where a reactive species is generated by actinic ray or radiation or by action of acid.

The above-described resin (A) preferably has a dissociable hydrogen atom. Examples of the dissociable hydrogen atom include a hydrogen atom in an OH group of the above-described acid group, and a hydrogen atom in the above-described alcoholic hydroxyl group.

As a preferred aspect, a moiety having the dissociable hydrogen atom may be a phenolic hydroxyl group.

That is, the above-described resin (A) preferably has a phenolic hydroxyl group. In addition, the above-described resin (A) preferably has a repeating unit having a phenolic hydroxyl group.

The resin (A) may have the acid group, the alcoholic hydroxyl group, or the acid-decomposable group, may have two of the acid group, the alcoholic hydroxyl group, or the acid-decomposable group, or may have all of the acid group, the alcoholic hydroxyl group, and the acid-decomposable group.

(Repeating Unit (a2) Having Acid-Decomposable Group)

The resin (A) may include a repeating unit having an acid-decomposable group (also referred to as a “repeating unit (a2)”).

The acid-decomposable group is as described above.

As the repeating unit having an acid-decomposable group, a repeating unit represented by Formula (A) is also preferable.

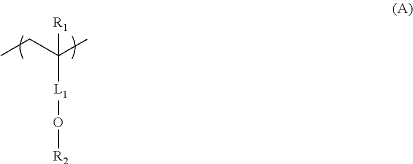

L₁ represents a divalent linking group which may have a fluorine atom or an iodine atom, R₁ represents a hydrogen atom, a fluorine atom, an iodine atom, a fluorine atom, an alkyl group which may have an iodine atom, or an aryl group which may have a fluorine atom or an iodine atom, and R₂ represents a leaving group which is eliminated by action of acid and may have a fluorine atom or an iodine atom. However, at least one of L₁, R₁, or R₂ has a fluorine atom or an iodine atom.

L₁ represents a divalent linking group which may have a fluorine atom or an iodine atom. Examples of the divalent linking group which may have a fluorine atom or an iodine atom include —CO—, —O—, —S—, —SO—, —SO₂—, a hydrocarbon group which may have a fluorine atom or an iodine atom (for example, an alkylene group, a cycloalkylene group, an alkenylene group, an arylene group, and the like), and a linking group formed by the linking of a plurality of these groups. Among these, L₁ is preferably —CO—, an arylene group, or -arylene group-alkylene group having a fluorine atom or an iodine atom-, and more preferably —CO— or -arylene group-alkylene group having a fluorine atom or an iodine atom-.

As the arylene group, a phenylene group is preferable.

The alkylene group may be linear or branched. The number of carbon atoms in the alkylene group is not particularly limited, but is preferably 1 to 10 and more preferably 1 to 3.

The total number of fluorine atoms and iodine atoms included in the alkylene group having a fluorine atom or an iodine atom is not particularly limited, but is preferably 2 or more, more preferably 2 to 10, and still more preferably 3 to 6.

R₁ represents a hydrogen atom, a fluorine atom, an iodine atom, an alkyl group which may have a fluorine atom or an iodine atom, or an aryl group which may have a fluorine atom or an iodine atom.

The alkyl group may be linear or branched. The number of carbon atoms in the alkyl group is not particularly limited, but is preferably 1 to 10 and more preferably 1 to 3.

The total number of fluorine atoms and iodine atoms included in the alkyl group having a fluorine atom or an iodine atom is not particularly limited, but is preferably 1 or more, more preferably 1 to 5, and still more preferably 1 to 3.

The above-described alkyl group may include a heteroatom such as an oxygen atom other than a halogen atom.

R₂ represents a leaving group which is eliminated by action of acid and may have a fluorine atom or an iodine atom. Examples of the leaving group which may have a fluorine atom or an iodine atom include leaving groups which are represented by Formulae (Y1) to (Y4) described above and have a fluorine atom or an iodine atom.

As the repeating unit having an acid-decomposable group, a repeating unit represented by Formula (AI) is also preferable.

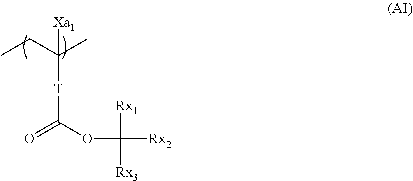

In Formula (AI),

Xa₁ represents a hydrogen atom, or an alkyl group which may have a substituent,

T represents a single bond or a divalent linking group, and

Rx₁ to Rx₃ each independently represent a (linear or branched) alkyl group, a (monocyclic or polycyclic) cycloalkyl group, a (linear or branched) alkenyl group, or a (monocyclic or polycyclic) aryl group, where, in a case where all of Rx₁ to Rx₃ are (linear or branched) alkyl groups, it is preferable that at least two of Rx₁, Rx₂, or Rx₃ are methyl groups, and

two of Rx₁ to Rx₃ may be bonded to each other to form a monocyclic ring or a polycyclic ring (a monocyclic or polycyclic cycloalkyl group and the like).

Examples of the alkyl group which may have a substituent, represented by Xa₁, include a methyl group and a group represented by —CH₂—R₁₁. R₁₁ represents a halogen atom (a fluorine atom or the like), a hydroxyl group, or a monovalent organic group, examples thereof include an alkyl group having 5 or less carbon atoms, which may be substituted with a halogen atom, an acyl group having 5 or less carbon atoms, which may be substituted with a halogen atom, and an alkoxy group having 5 or less carbon atoms, which may be substituted with a halogen atom. Among these, an alkyl group having 3 or less carbon atoms is preferable, and a methyl group is more preferable. Xa₁ is preferably a hydrogen atom, a methyl group, a trifluoromethyl group, or a hydroxymethyl group.

Examples of the divalent linking group of T include an alkylene group, an aromatic ring group, a —COO—Rt- group, and an —O—Rt- group. In the formulae, Rt represents an alkylene group or a cycloalkylene group.

T is preferably a single bond or a —COO—Rt- group. In a case where T represents a —COO—Rt-group, Rt is preferably an alkylene group having 1 to 5 carbon atoms, and more preferably a —CH₂— group, a —(CH₂)₂— group, or a —(CH₂)₃— group.

As the alkyl group of Rx₁ to Rx₃, an alkyl group having 1 to 4 carbon atoms, such as a methyl group, an ethyl group, an n-propyl group, an isopropyl group, an n-butyl group, an isobutyl group, and a t-butyl group, is preferable.

As the cycloalkyl group of Rx₁ to Rx₃, a monocyclic cycloalkyl group such as a cyclopentyl group and a cyclohexyl group, or a polycyclic cycloalkyl group such as a norbornyl group, a tetracyclodecanyl group, a tetracyclododecanyl group, and an adamantyl group is preferable.

As the aryl group of Rx₁ to Rx₃, an aryl group having 6 to 14 carbon atoms is preferable and an aryl group having 6 to 10 carbon atoms is more preferable, and examples thereof include a phenyl group, a naphthyl group, and an anthryl group.

As the alkenyl group of Rx₁ to Rx₃, a vinyl group is preferable.

As the cycloalkyl group formed by bonding two of Rx₁ to Rx₃, a monocyclic cycloalkyl group such as a cyclopentyl group and a cyclohexyl group is preferable. In addition, a polycyclic cycloalkyl group such as a norbornyl group, a tetracyclodecanyl group, a tetracyclododecanyl group, and an adamantyl group is preferable. Among these, a monocyclic cycloalkyl group having 5 or 6 carbon atoms is preferable.

In the cycloalkyl group formed by the bonding of two of Rx₁ to Rx₃, for example, one of methylene groups constituting the ring may be replaced with a heteroatom such as an oxygen atom, with a group including a heteroatom, such as a carbonyl group, or with a vinylidene group. In addition, in the cycloalkyl group, one or more of the ethylene groups constituting the cycloalkane ring may be replaced with a vinylene group.

With regard to the repeating unit represented by Formula (AI), for example, an aspect in which Rx₁ is a methyl group or an ethyl group and Rx₂ and Rx₃ are bonded to each other to form the above-described cycloalkyl group is preferable.

In a case where each of the above-described groups has a substituent, examples of the substituent include an alkyl group (having 1 to 4 carbon atoms), a halogen atom, a hydroxyl group, an alkoxy group (having 1 to 4 carbon atoms), a carboxyl group, and an alkoxycarbonyl group (having 2 to 6 carbon atoms). The substituent preferably has 8 or less carbon atoms.

As a preferred aspect, it is preferable that two of Rx₁ to Rx₃ are to each other to form a monocyclic ring or a polycyclic ring (a monocyclic or polycyclic cycloalkyl group and the like).

The repeating unit represented by Formula (AI) is preferably an acid-decomposable tertiary alkyl (meth)acrylate ester-based repeating unit (a repeating unit in which Xa₁ represents a hydrogen atom or a methyl group and T represents a single bond).

Specific examples of the repeating unit having an acid-decomposable group are shown below, but the present invention is not limited thereto. In the formulae, Xa₁ represents H, CH₃, CF₃, or CH₂OH, and Rxa and Rxb each independently represent a linear or branched alkyl group having 1 to 5 carbon atoms.

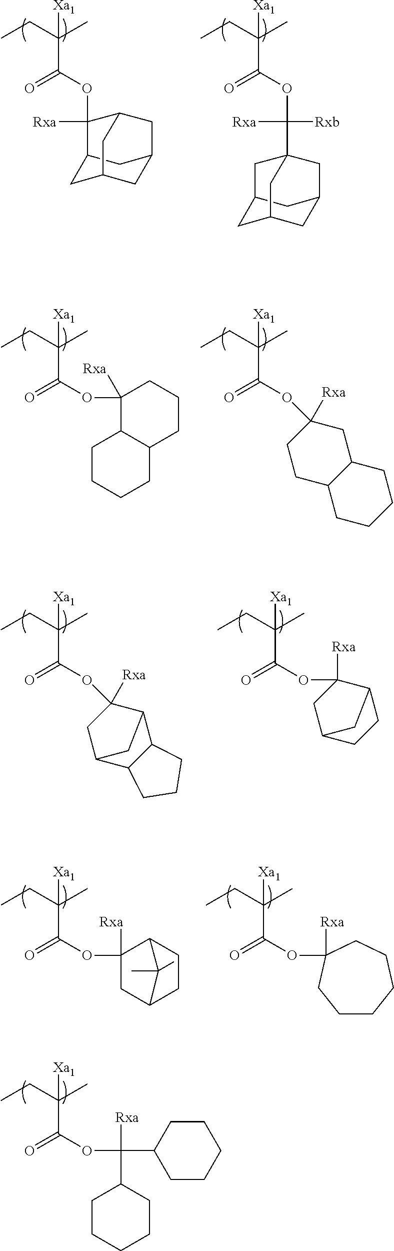

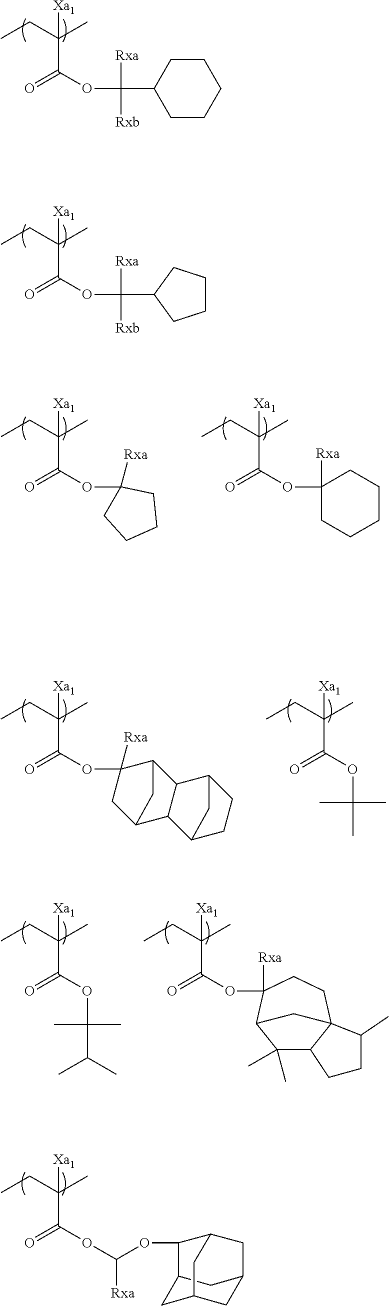

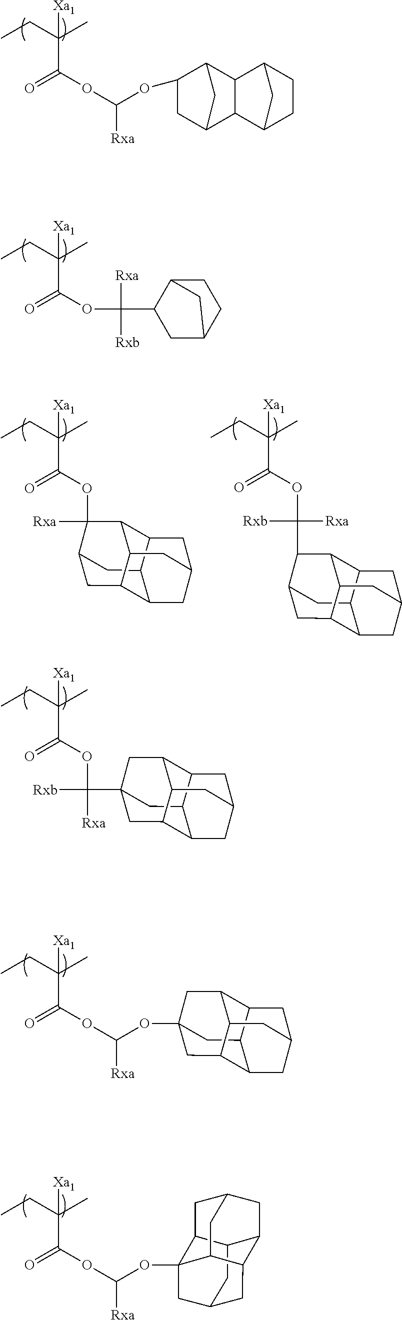

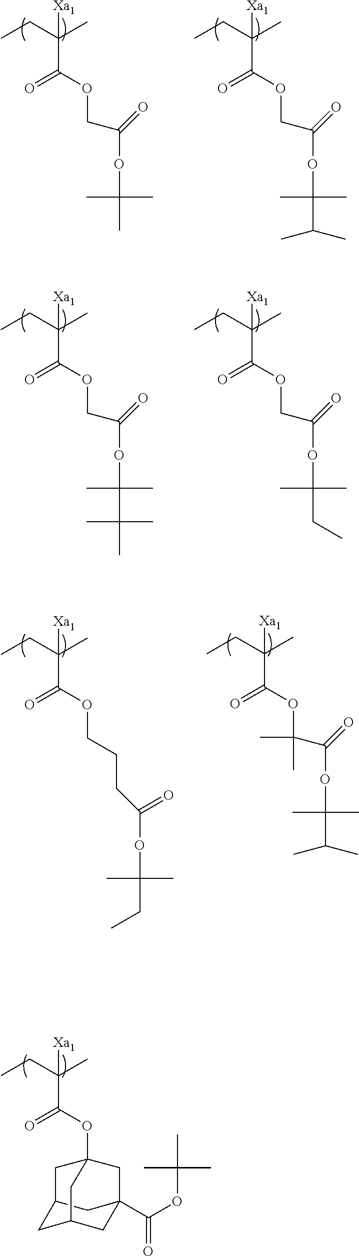

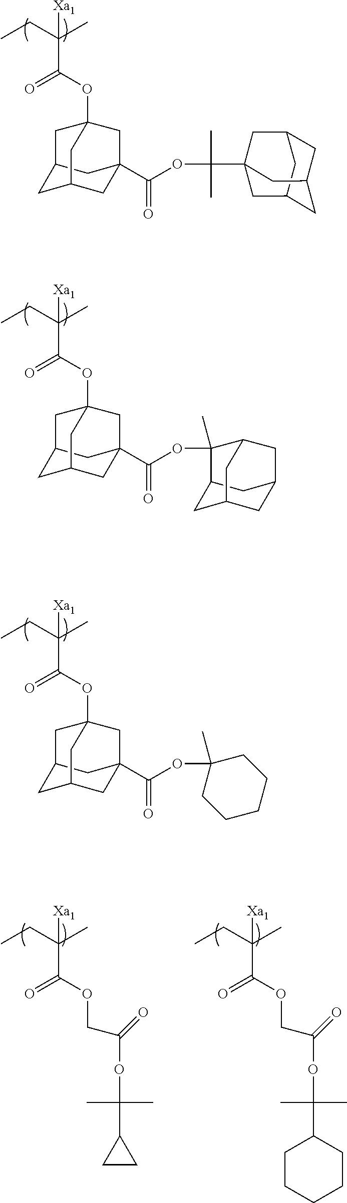

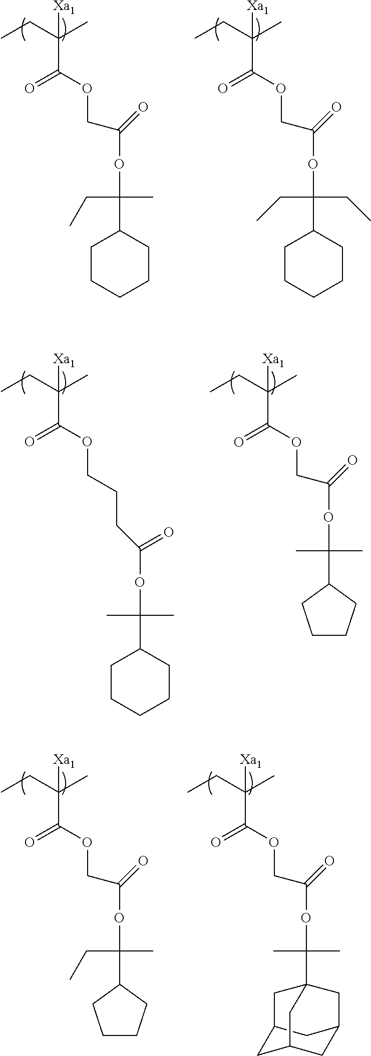

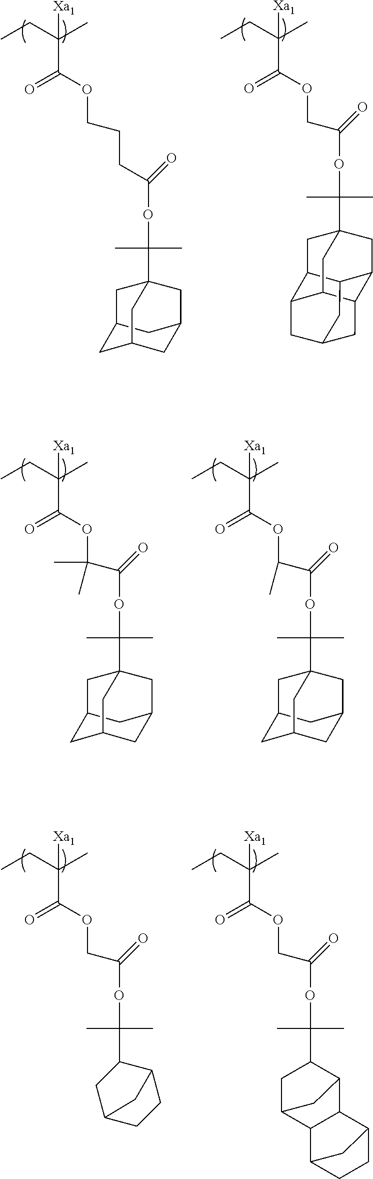

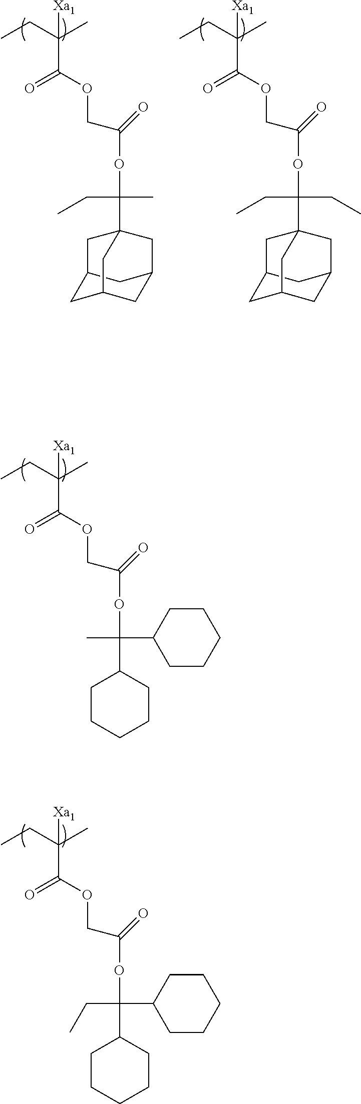

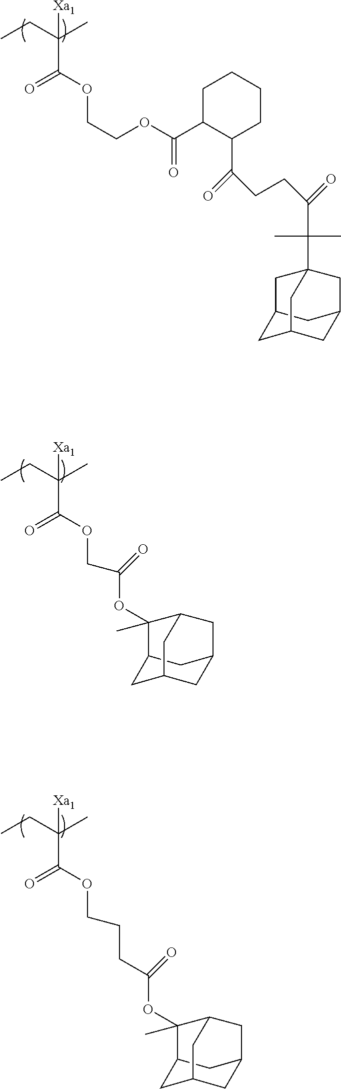

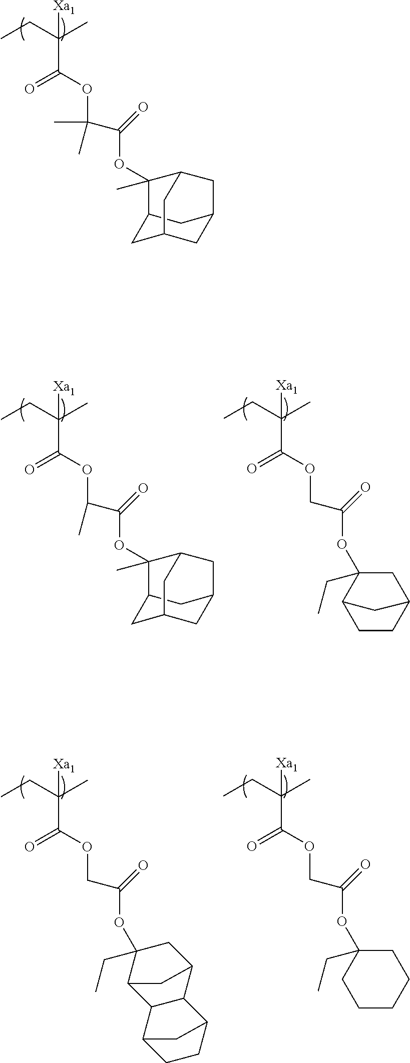

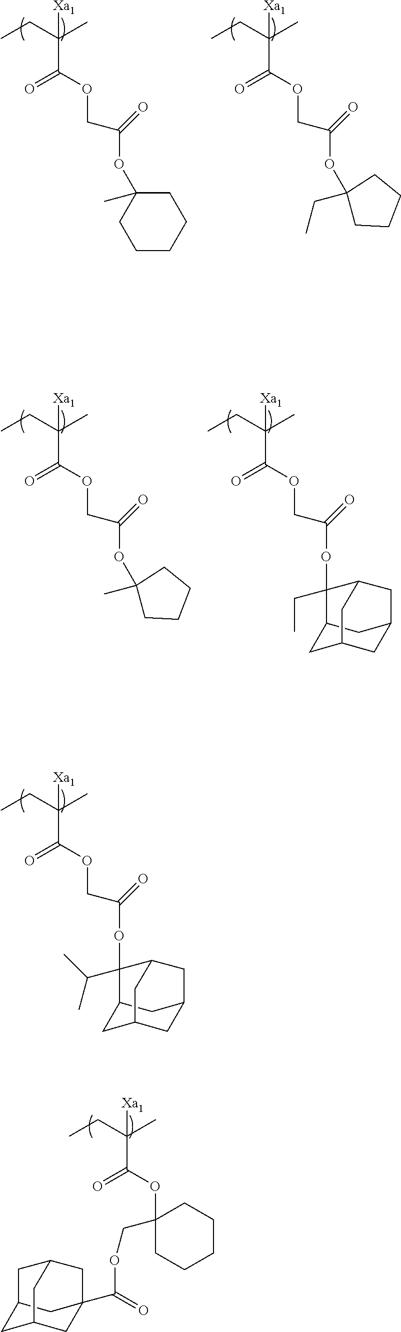

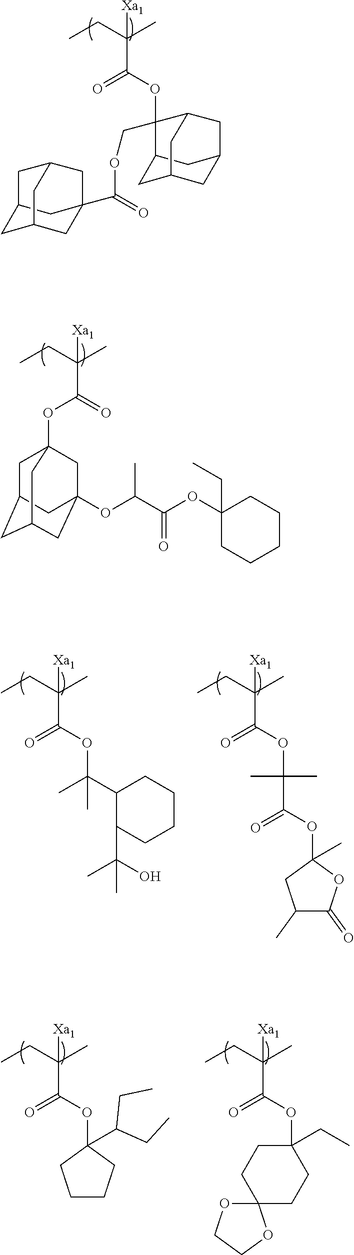

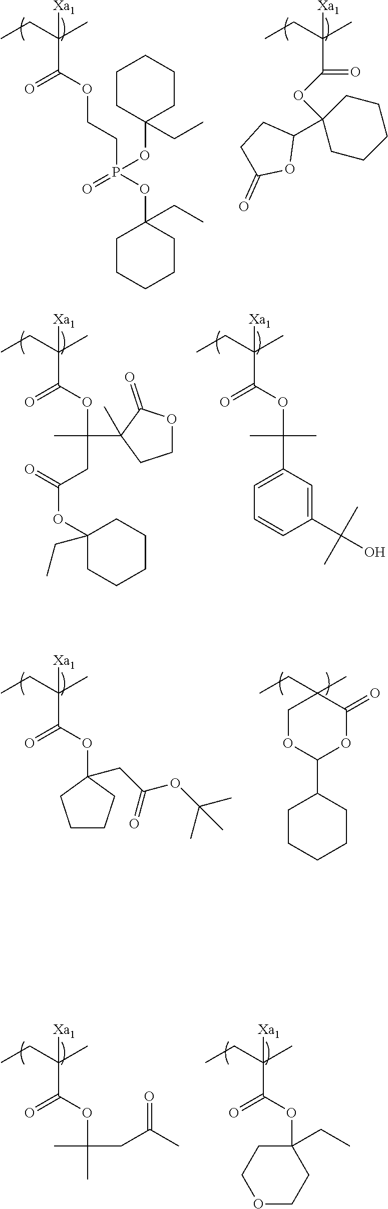

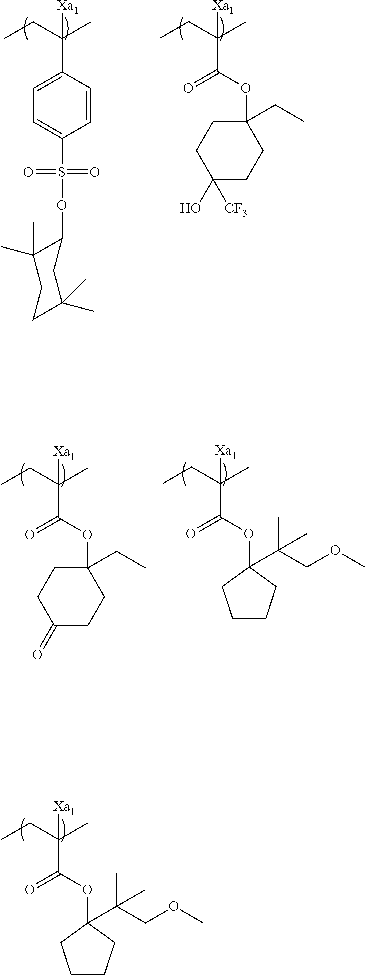

The resin (A) may have, as the repeating unit having an acid-decomposable group, a repeating unit which has an acid-decomposable group including an unsaturated bond.

As the repeating unit having an acid-decomposable group including an unsaturated bond, a repeating unit represented by Formula (B) is preferable.

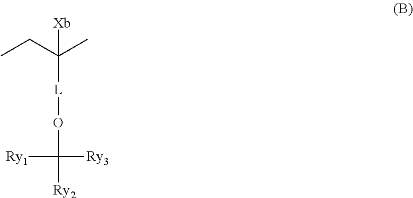

In Formula (B), Xb represents a hydrogen atom, a halogen atom, or an alkyl group which may have a substituent, L represents a single bond or a divalent linking group which may have a substituent, and Ry₁ to Ry₃ each independently represent a linear or branched alkyl group, a monocyclic or polycyclic cycloalkyl group, an alkenyl group, an alkynyl group, or a monocyclic or polycyclic aryl group, where, at least one of Ry₁, Ry₂, or Ry₃ represents an alkenyl group, an alkynyl group, a monocyclic or polycyclic cycloalkenyl group, or a monocyclic or polycyclic aryl group, and

two of Ry₁ to Ry₃ may be bonded to each other to form a monocycle or a polycycle (monocyclic or polycyclic cycloalkyl group, cycloalkenyl group, or the like).

Examples of the alkyl group which may have a substituent, represented by Xb, include a methyl group and a group represented by —CH₂—R₁₁. R₁₁ represents a halogen atom (a fluorine atom or the like), a hydroxyl group, or a monovalent organic group, examples thereof include an alkyl group having 5 or less carbon atoms, which may be substituted with a halogen atom, an acyl group having 5 or less carbon atoms, which may be substituted with a halogen atom, and an alkoxy group having 5 or less carbon atoms, which may be substituted with a halogen atom. Among these, an alkyl group having 3 or less carbon atoms is preferable, and a methyl group is more preferable. Xb is preferably a hydrogen atom, a fluorine atom, a methyl group, a trifluoromethyl group, or a hydroxymethyl group.

Examples of the divalent linking group of L include a —Rt- group, a —CO— group, a —COO—Rt- group, a —COO—Rt-CO— group, a —Rt-CO— group, and an —O—Rt- group. In the formulae, Rt represents an alkylene group, a cycloalkylene group, or an aromatic ring group, and an aromatic ring group is preferable.
L is preferably a -Rt- group, a —CO— group, a —COO-Rt-CO— group, or a —Rt-CO— group. Rt is preferably an aromatic group which may have a substituent such as a halogen atom, a hydroxyl group, and an alkoxy group.

As the alkyl group of Ry₁ to Ry₃, an alkyl group having 1 to 4 carbon atoms, such as a methyl group, an ethyl group, an n-propyl group, an isopropyl group, an n-butyl group, an isobutyl group, and a t-butyl group, is preferable.

As the cycloalkyl group of Ry₁ to Ry₃, a monocyclic cycloalkyl group such as a cyclopentyl group and a cyclohexyl group, or a polycyclic cycloalkyl group such as a norbornyl group, a tetracyclodecanyl group, a tetracyclododecanyl group, and an adamantyl group is preferable.

As the aryl group of Ry₁ to Ry₃, an aryl group having 6 to 10 carbon atoms is preferable, and examples thereof include a phenyl group, a naphthyl group, and an anthryl group.

As the alkenyl group of Ry₁ to Ry₃, a vinyl group is preferable.

As the alkynyl group of Ry₁ to Ry₃, an ethynyl group is preferable.

As the cycloalkenyl group of Ry₁ to Ry₃, a structure in which a part of a monocyclic cycloalkyl group such as a cyclopentyl group and a cyclohexyl group includes a double bond is preferable.

As the cycloalkyl group formed by the bonding of two of Ry₁ to Ry₃, a monocyclic cycloalkyl group such as a cyclopentyl group and a cyclohexyl group, or a polycyclic cycloalkyl group such as a norbornyl group, a tetracyclodecanyl group, a tetracyclododecanyl group, and an adamantyl group is preferable. Among these, a monocyclic cycloalkyl group having 5 or 6 carbon atoms is more preferable.

In the cycloalkyl group or cycloalkenyl group formed by the bonding of two of Ry₁ to Ry₃, for example, one of methylene groups constituting the ring may be replaced with a heteroatom such as an oxygen atom, with a group including a heteroatom, such as a carbonyl group, a —SO₂— group, and a —SO₃— group, with a vinylidene group, or with a combination of these groups. In addition, in the cycloalkyl group or cycloalkenyl group, one or more of the ethylene groups constituting the cycloalkane ring or cycloalkene ring may be replaced with a vinylene group.

With regard to the repeating unit represented by Formula (B), for example, an aspect in which Ry₁ is a methyl group, an ethyl group, a vinyl group, an allyl group, or an aryl group and Ry₂ and Ry₃ are bonded to each other to form the above-described cycloalkyl group or cycloalkenyl group is preferable.

In a case where each of the above-described groups has a substituent, examples of the substituent include an alkyl group (having 1 to 4 carbon atoms), a halogen atom, a hydroxyl group, an alkoxy group (having 1 to 4 carbon atoms), a carboxyl group, and an alkoxycarbonyl group (having 2 to 6 carbon atoms). The substituent preferably has 8 or less carbon atoms.

The repeating unit represented by Formula (B) is preferably an acid-decomposable (meth)acrylic acid tertiary ester-based repeating unit (repeating unit in which Xb represents a hydrogen atom or a methyl group and L represents a —CO— group), an acid-decomposable hydroxystyrene tertiary alkyl ether-based repeating unit (repeating unit in which Xb represents a hydrogen atom or a methyl group and L represents a phenylene group), or an acid-decomposable styrene carboxylic acid tertiary ester-based repeating unit (repeating unit in which Xb represents a hydrogen atom or a methyl group and L represents a —Rt-CO— group (Rt is an aromatic group)).

A content of the repeating unit having an acid-decomposable group including an unsaturated bond is preferably 15% by mole or more, more preferably 20% by mole or more, and still more preferably 30% by mole or more with respect to all repeating units in the resin (A). In addition, the upper limit value thereof is preferably 80% by mole or less, more preferably 70% by mole or less, and particularly preferably 60% by mole or less with respect to all repeating units in the resin (A).

Specific examples of the repeating unit having an acid-decomposable group including an unsaturated bond are shown below, but the present invention is not limited thereto. In the formulae, Xb and L₁ represent any of the above-described substituents or linking groups; Ar represents an aromatic group; R represents a substituent such as a hydrogen atom, an alkyl group, a cycloalkyl group, an aryl group, an aralkyl group, an alkenyl group, a hydroxyl group, an alkoxy group, an acyloxy group, a cyano group, a nitro group, an amino group, a halogen atom, an ester group (—OCOR′″ or —COOR′″; R′″ is an alkyl group or fluorinated alkyl group having 1 to 20 carbon atoms), and a carboxyl group; R′ represents a linear or branched alkyl group, a monocyclic or polycyclic cycloalkyl group, an alkenyl group, an alkynyl group, or a monocyclic or polycyclic aryl group; Q represents a heteroatom such as an oxygen atom, a group containing a heteroatom, such as a carbonyl group, —SO₂— group, and —SO₃— group, a vinylidene group, or a combination thereof, and n and m represent an integer of 0 or more.

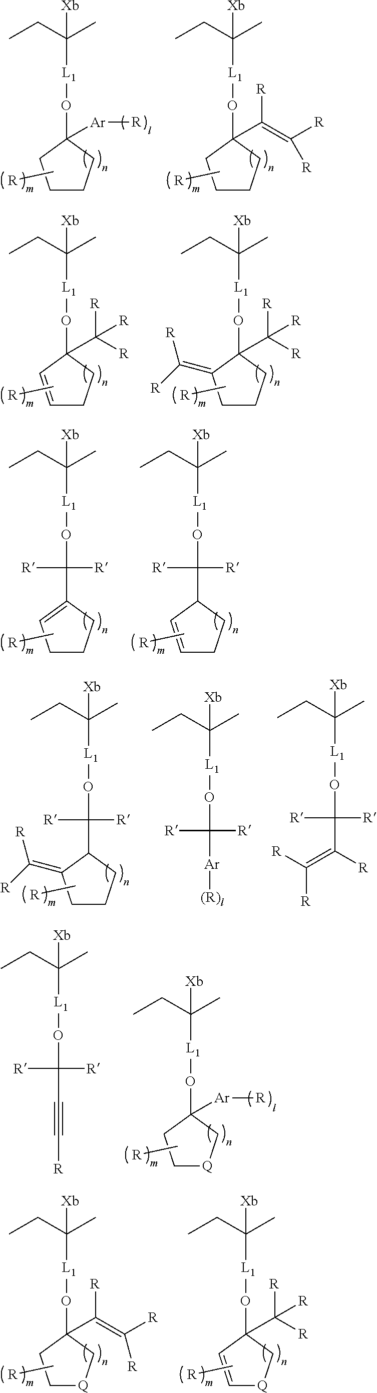

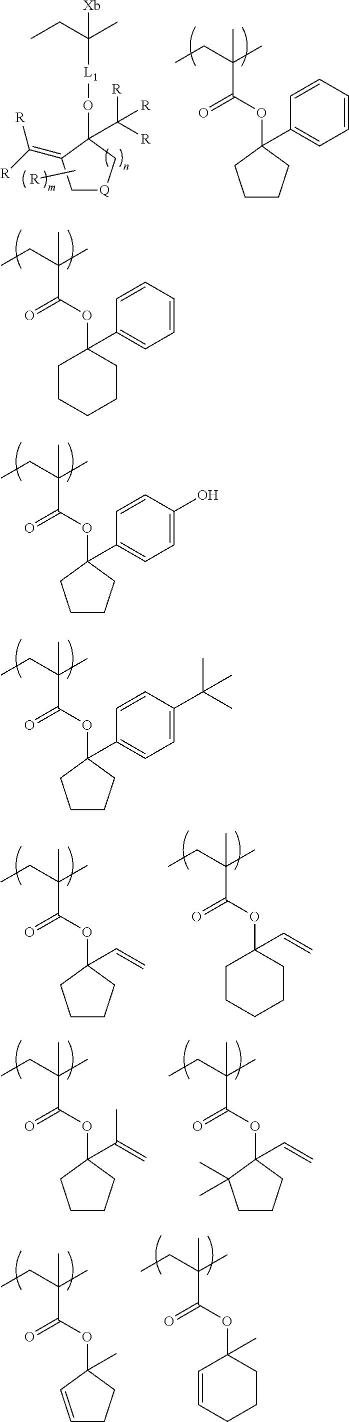

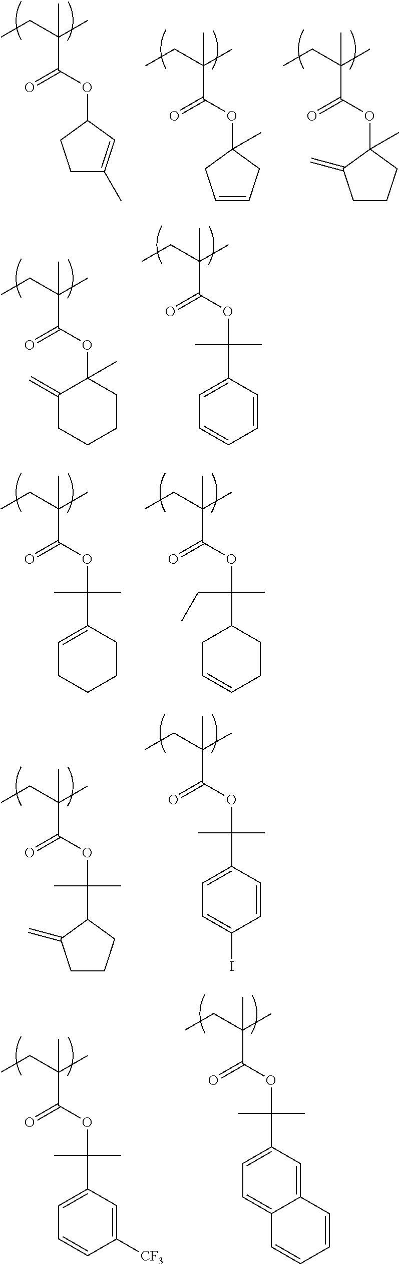

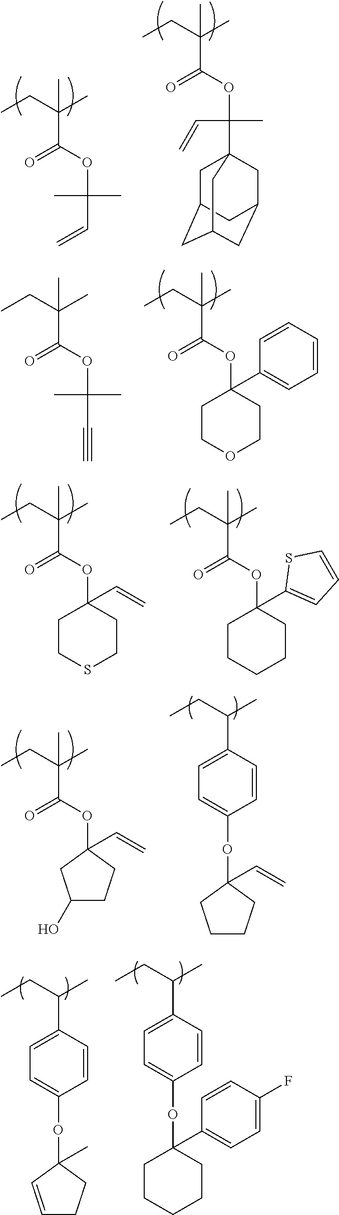

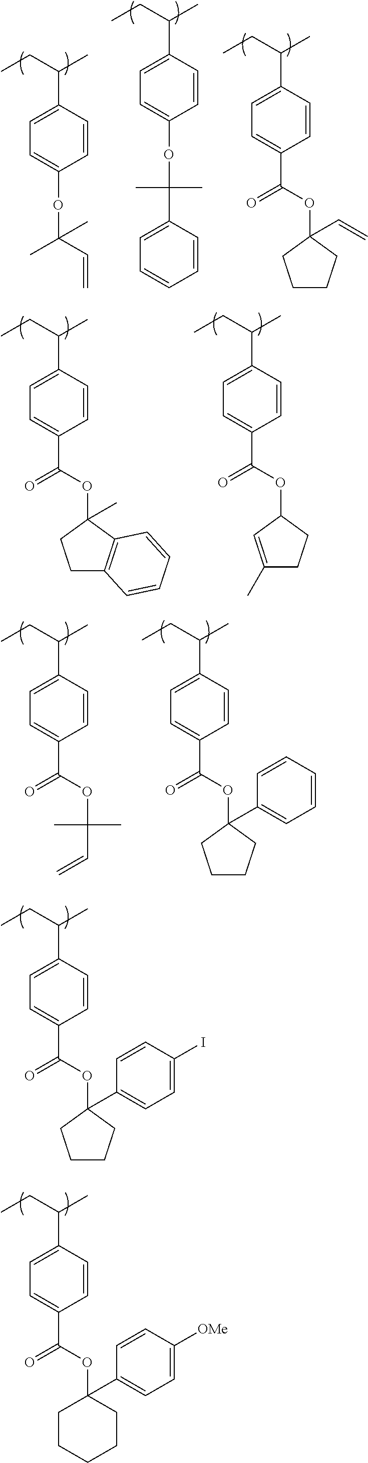

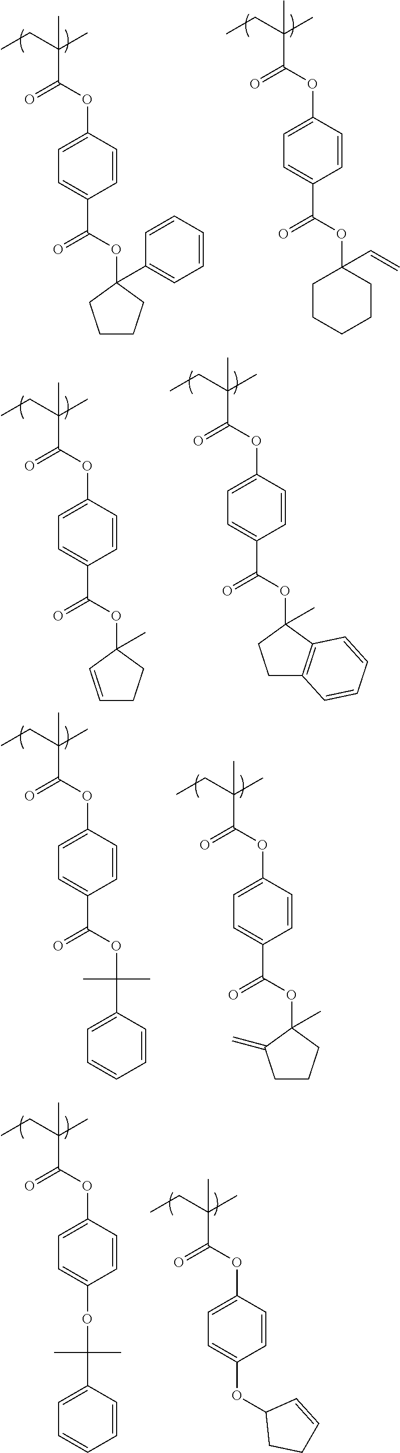

A content of the repeating unit having an acid-decomposable group is preferably 15% by mole or more, more preferably 20% by mole or more, and still more preferably 30% by mole or more with respect to all repeating units in the resin (A). In addition, the upper limit value thereof is preferably 90% by mole or less, more preferably 80% by mole or less, still more preferably 70% by mole or less, and particularly preferably 60% by mole or less with respect to all repeating units in the resin (A).

The resin (A) may include at least one repeating unit selected from the group consisting of the following group A and/or at least one repeating unit selected from the group consisting of the following group B.

Group A: group consisting of the following repeating units (20) to (29)
(20) repeating unit having an acid group, which will be described later
(21) repeating unit having neither an acid-decomposable group nor an acid group and having a fluorine atom, a bromine atom, or an iodine atom, which will be described later
(22) repeating unit having a lactone group, a sultone group, or a carbonate group, which will be described later
(23) repeating unit having a photoacid generating group, which will be described later
(24) repeating unit represented by Formula (V-1) or Formula (V-2), which will be described later
(25) repeating unit represented by Formula (A), which will be described later
(26) repeating unit represented by Formula (B), which will be described later
(27) repeating unit represented by Formula (C), which will be described later
(28) repeating unit represented by Formula (D), which will be described later
(29) repeating unit represented by Formula (E), which will be described later

Group B: group consisting of the following repeating units (30) to (32)
(30) repeating unit having at least one group selected from a lactone group, a sultone group, a carbonate group, a hydroxyl group, a cyano group, or an alkali-soluble group, which will be described later
(31) repeating unit having an alicyclic hydrocarbon structure and not exhibiting acid decomposability, which will be described later
(32) repeating unit represented by Formula (III) having neither a hydroxyl group nor a cyano group, which will be described later

The resin (A) preferably has an acid group, and preferably includes a repeating unit having an acid group. The definition of the acid group will be described later together with a suitable aspect of the repeating unit having an acid group. In a case where the resin (A) has an acid group, interaction the resin (A) and the acid generated from the photoacid generator is more excellent. As a result, diffusion of the acid is further suppressed, and a cross-sectional shape of the formed pattern can be more rectangular.

In a case where the composition according to the embodiment of the present invention is used as an actinic ray-sensitive or radiation-sensitive resin composition with EUV, it is preferable that the resin (A) has at least one repeating unit selected from the group consisting of the above-described group A.

In addition, in a case where the composition according to the embodiment of the present invention is used as an actinic ray-sensitive or radiation-sensitive resin composition with EUV, it is preferable that the resin (A) includes at least one of a fluorine atom or an iodine atom. In a case where the resin (A) includes both a fluorine atom and an iodine atom, the resin (A) may have one repeating unit including both a fluorine atom and an iodine atom, and the resin (A) may include two kinds of repeating units, that is, a repeating unit having a fluorine atom and a repeating unit having an iodine atom.

In addition, in a case where the composition according to the embodiment of the present invention is used as an actinic ray-sensitive or radiation-sensitive resin composition with EUV, it is also preferable that the resin (A) has a repeating unit having an aromatic group.

In a case where the composition according to the embodiment of the present invention is used as an actinic ray-sensitive or radiation-sensitive resin composition with ArF, it is preferable that the resin (A) has at least one repeating unit selected from the group consisting of the above-described group B.

In a case where the composition according to the embodiment of the present invention is used as an actinic ray-sensitive or radiation-sensitive resin composition with ArF, it is preferable that the resin (A) does not include a fluorine atom and a silicon atom.

In addition, in a case where the composition according to the embodiment of the present invention is used as an actinic ray-sensitive or radiation-sensitive resin composition with ArF, it is preferable that the resin (A) does not have an aromatic group.

(Repeating Unit Having Acid Group)

The resin (A) may have a repeating unit having an acid group.

The acid group is as described above.

In a case where the resin (A) has an acid group having a pKa of 13 or less, a content of the acid group in the resin (A) is not particularly limited, but is usually 0.2 to 6.0 mmol/g. Among these, 0.8 to 6.0 mmol/g is preferable, 1.2 to 5.0 mmol/g is more preferable, and 1.6 to 4.0 mmol/g is still more preferable. In a case where the content of the acid group is within the above-described range, the development proceeds satisfactorily, the formed pattern shape is excellent, and the resolution is also excellent.

The repeating unit having an acid group is preferably a repeating unit different from the repeating unit having the structure in which a polar group is protected by the leaving group which is eliminated by action of acid as described above, and the repeating unit having a lactone group, a sultone group, or a carbonate group, which will be described later.

The repeating unit having an acid group may have a fluorine atom or an iodine atom.

Examples of the repeating unit having an acid group include the following repeating units.

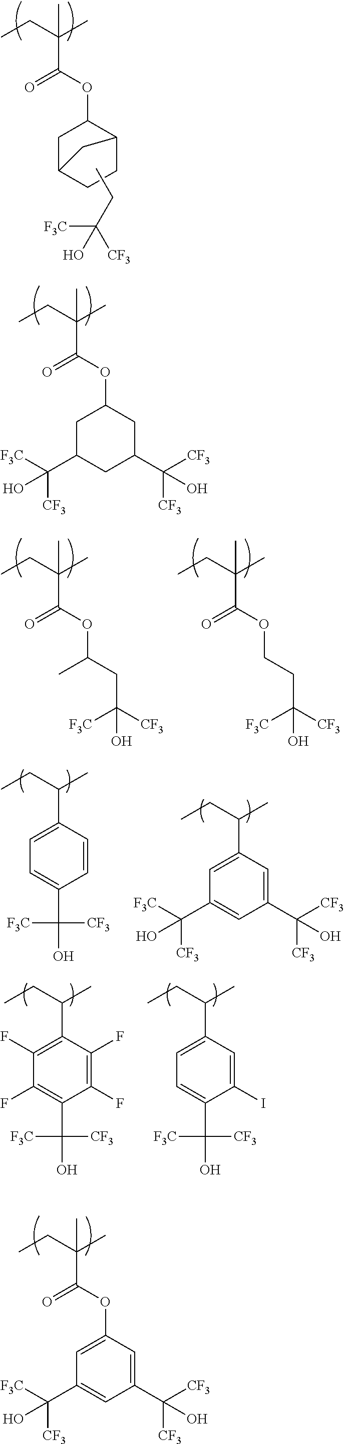

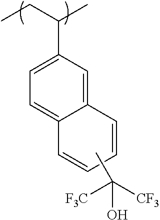

As the repeating unit having an acid group, a repeating unit represented by Formula (1) is preferable.

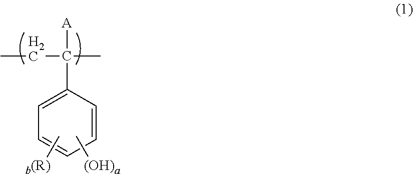

In Formula (1), A represents a hydrogen atom, an alkyl group, a cycloalkyl group, a halogen atom, or a cyano group, R represents a halogen atom, an alkyl group, a cycloalkyl group, an aryl group, an alkenyl group, an aralkyl group, an alkoxy group, an alkylcarbonyloxy group, an alkylsulfonyloxy group, an alkyloxycarbonyl group, or an aryloxycarbonyl group, where in a case of a plurality of R's, the plurality of R's may be the same or different from each other, in a case where a plurality of R's are present, the plurality of R's may be combined with each other to form a ring, R is preferably a hydrogen atom. a represents an integer of 1 to 3, and b represents an integer of 0 to (5-a).

The repeating unit having an acid group is exemplified below. In the formulae, a represents an integer of 1 to 3.

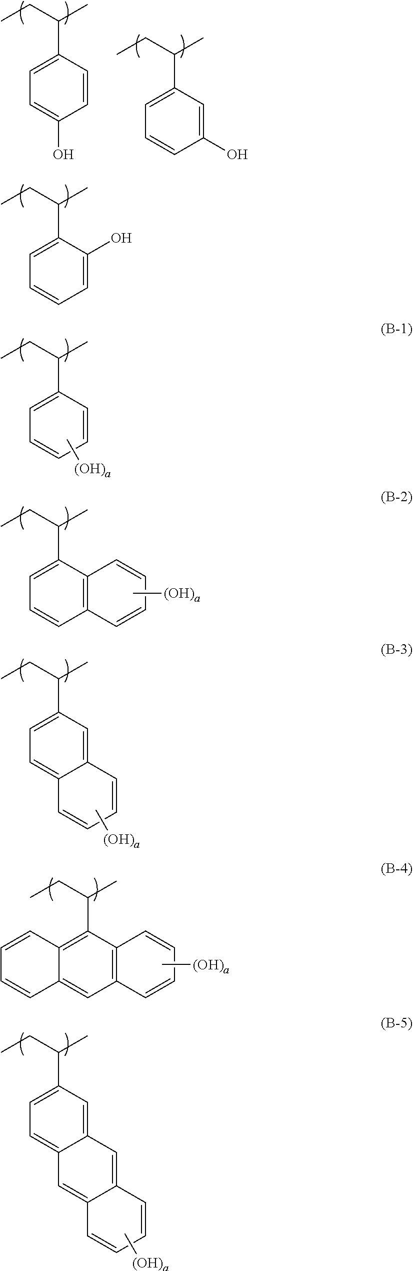

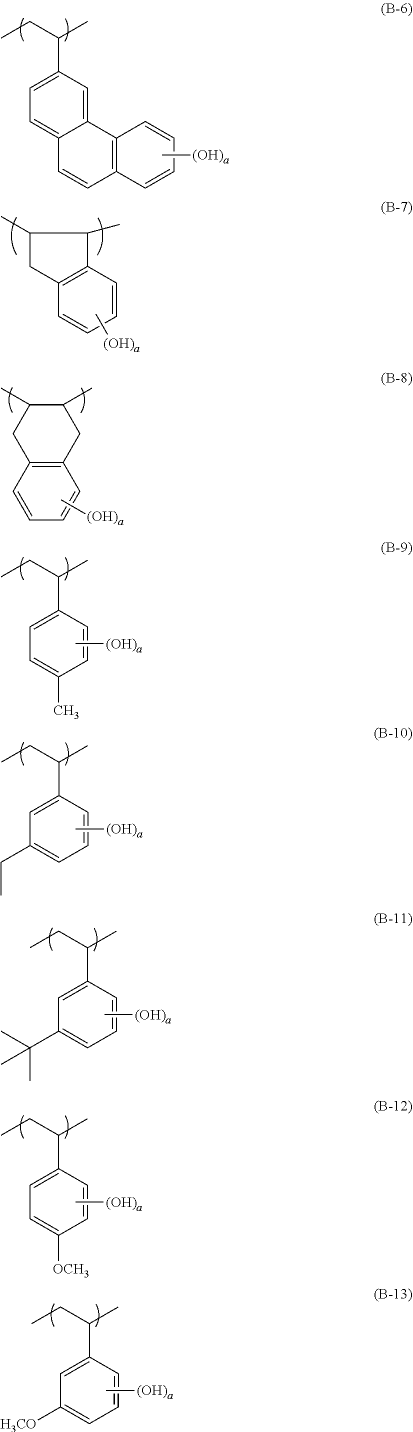

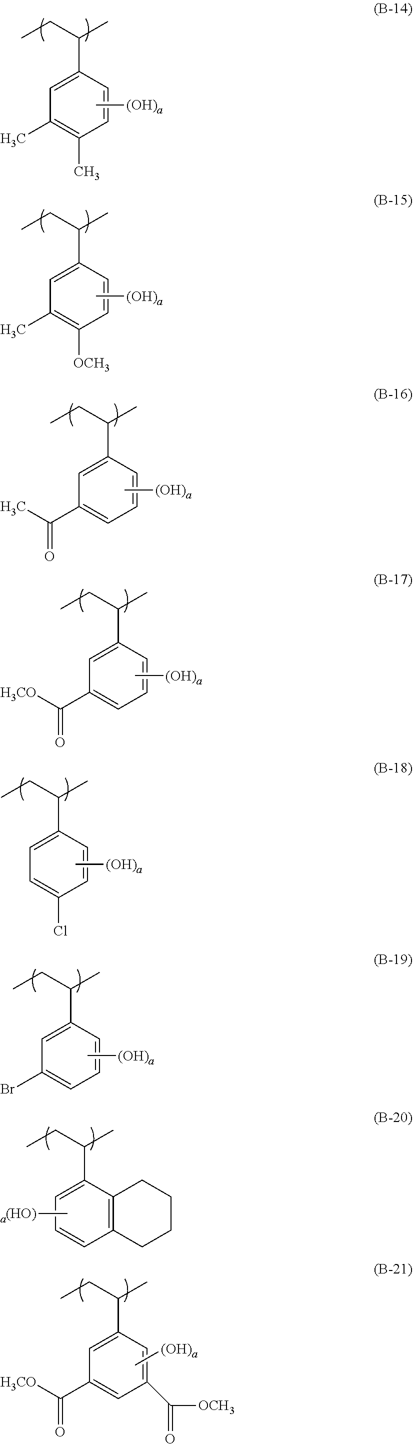

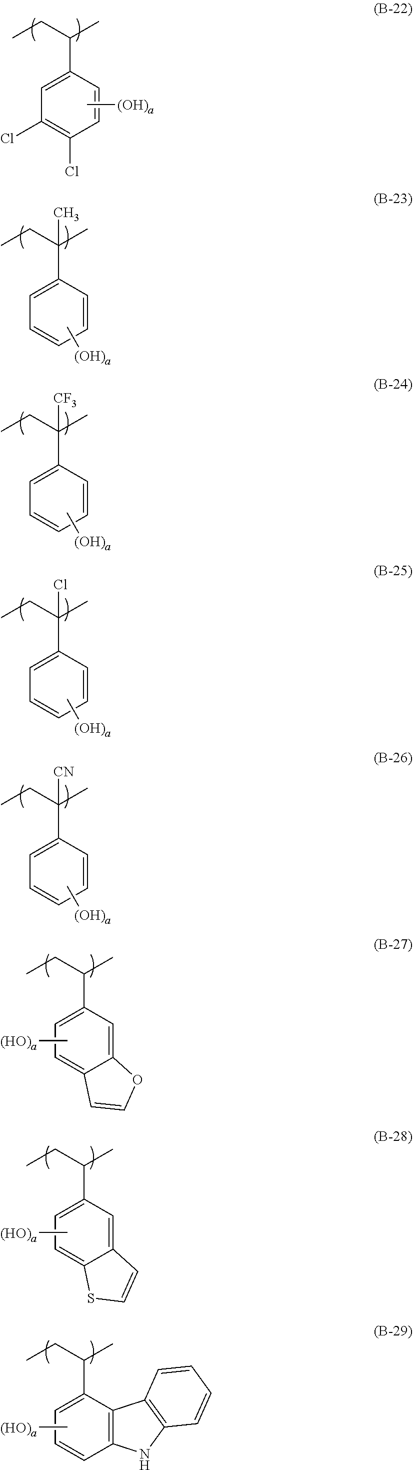

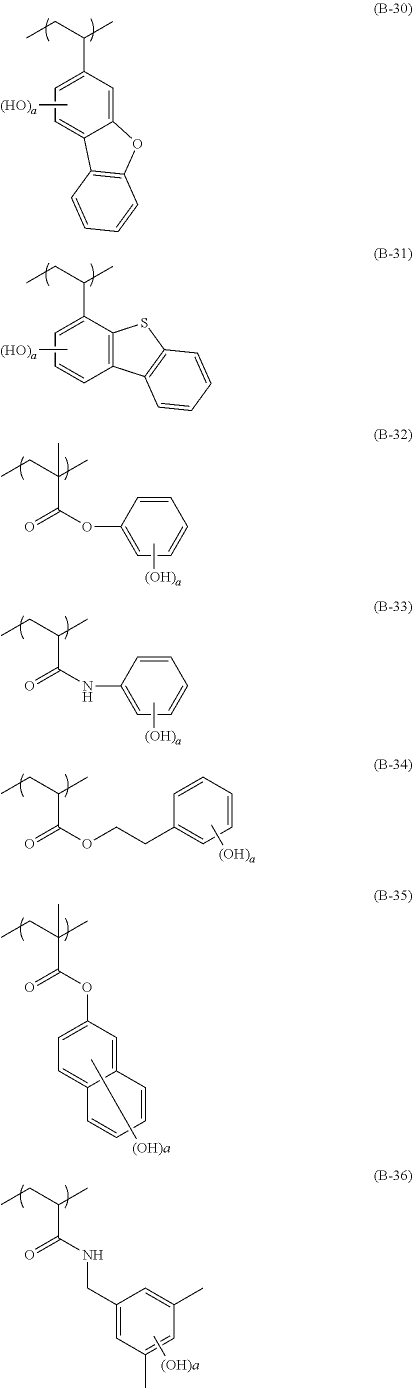

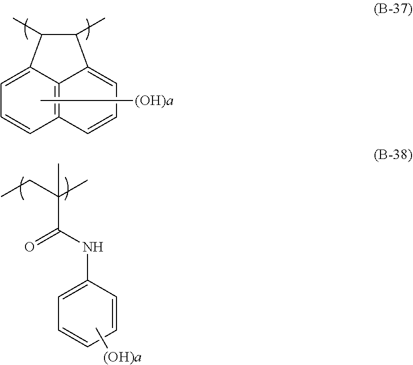

Among the above-described repeating units, repeating units specifically shown below are preferable. In the formulae, R represents a hydrogen atom or a methyl group, and a represents an integer of 1 to 3.

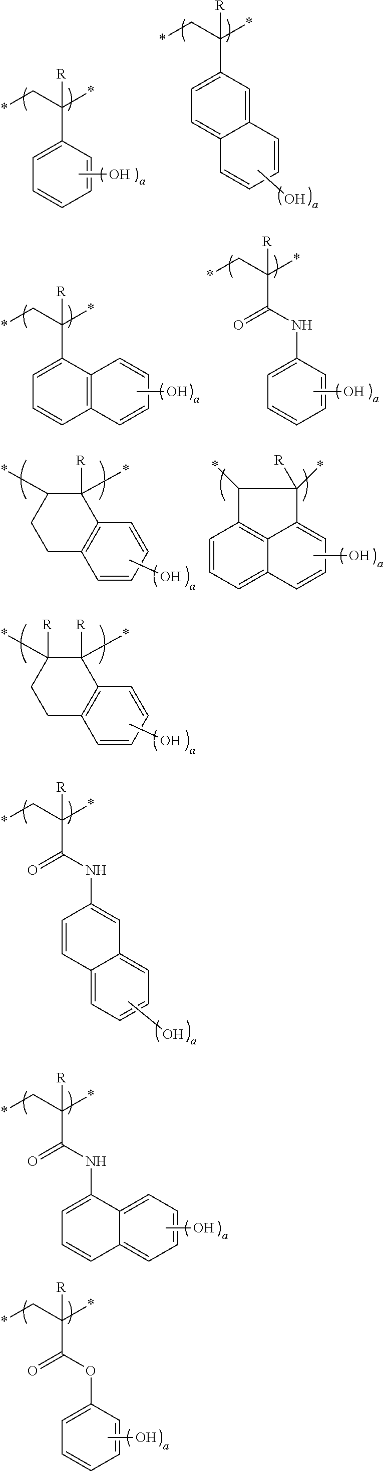

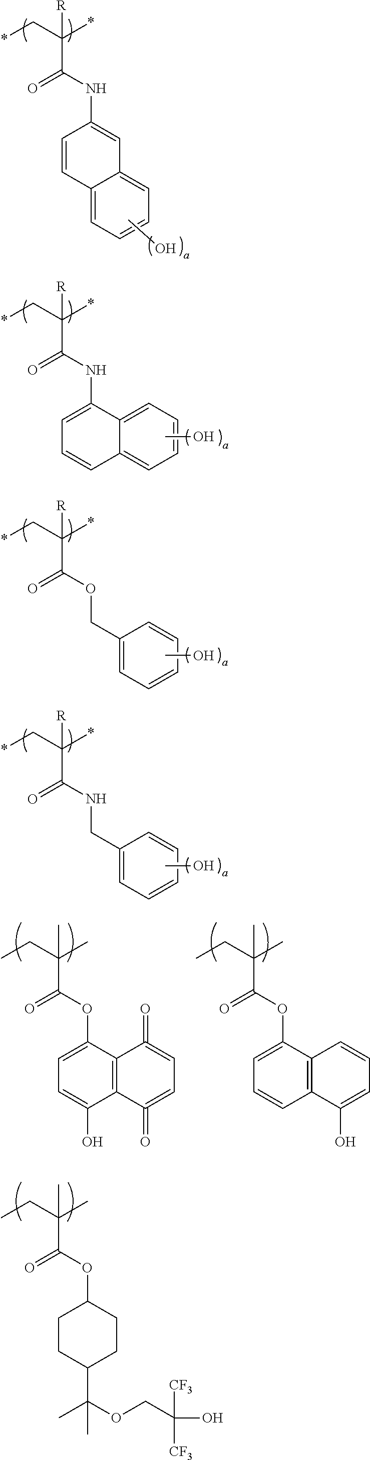

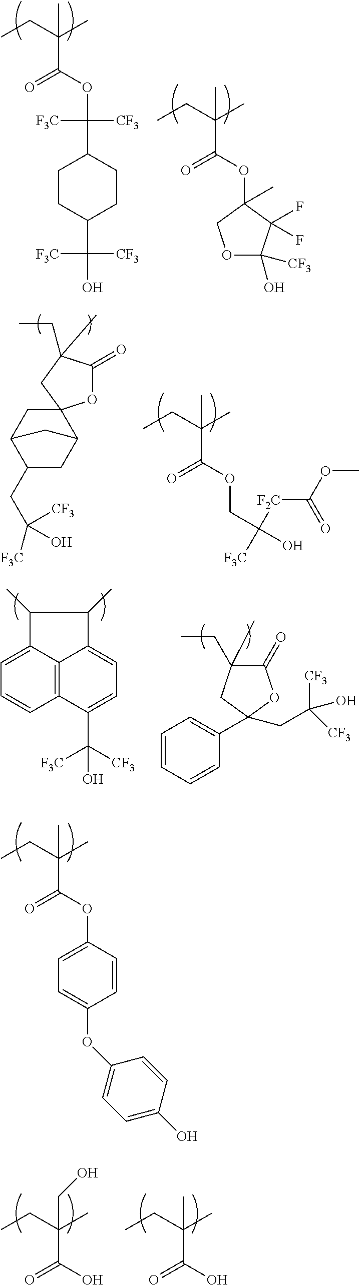

A content of the repeating unit having an acid group is preferably 10% by mole or more, and more preferably 15% by mole or more with respect to all repeating units in the resin (A). In addition, the upper limit value thereof is preferably 70% by mole or less, more preferably 65% by mole or less, and still more preferably 60% by mole or less with respect to all repeating units in the resin (A).

(Repeating Unit Having Neither Acid-Decomposable Group Nor Acid Group and Having Fluorine Atom, Bromine Atom, or Iodine Atom)

The resin (A) may have a repeating unit having neither an acid-decomposable group nor an acid group and having a fluorine atom, a bromine atom, or an iodine atom (hereinafter, also referred to as a unit X), in addition to the above-described <repeating unit having acid-decomposable group> and <repeating unit having acid group>. In addition, it is preferable that the <repeating unit having neither an acid-decomposable group nor an acid group and having a fluorine atom, a bromine atom, or an iodine atom> herein is different from other types of the repeating units belonging to the group A, such as <repeating unit having lactone group, sultone group, or carbonate group> and <repeating unit having photoacid generating group> described later.

As the unit X, a repeating unit represented by Formula (C) is preferable.

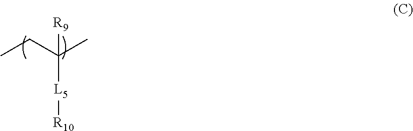

L₅ represents a single bond or an ester group. R₉ represents a hydrogen atom or an alkyl group which may have a fluorine atom or an iodine atom. R₁₀ represents a hydrogen atom, an alkyl group which may have a fluorine atom or an iodine atom, a cycloalkyl group which may have a fluorine atom or an iodine atom, an aryl group which may have a fluorine atom or an iodine atom, or a group formed by a combination thereof.

The repeating unit having a fluorine atom, a bromine atom, or an iodine atom is exemplified below.

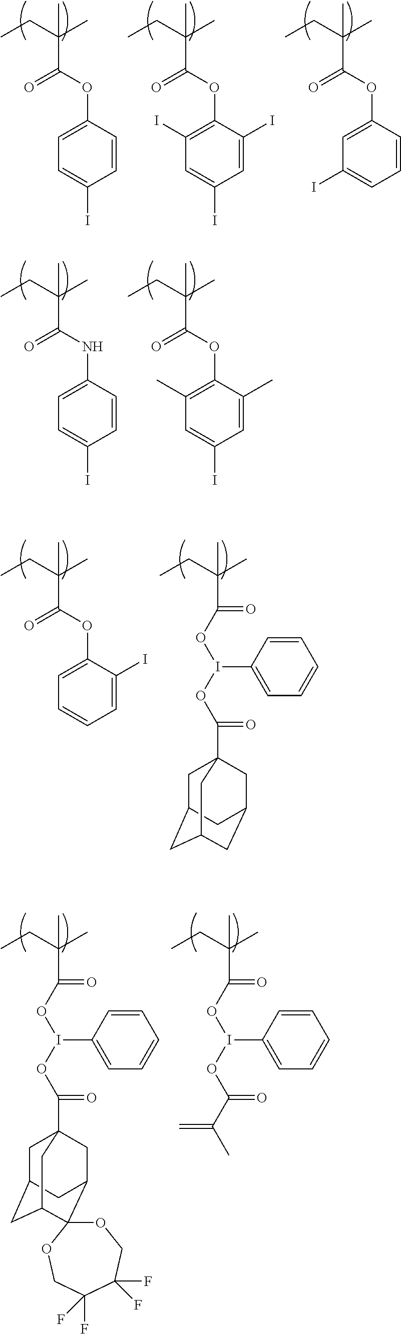

A content of the unit X a is preferably 0% by mole or more, more preferably 5% by mole or more, and still more preferably 10% by mole or more with respect to all repeating units in the resin (A). In addition, the upper limit value thereof is preferably 50% by mole or less, more preferably 45% by mole or less, and still more preferably 40% by mole or less with respect to all repeating units in the resin (A).

The total content of the repeating unit including at least any one of a fluorine atom, a bromine atom, or an iodine atom in the repeating units of the resin (A) is preferably 10% by mole or more, more preferably 20% by mole or more, still more preferably 30% by mole or more, and particularly preferably 40% by mole or more with respect to all repeating units of the resin (A). The upper limit value thereof is not particularly limited, and is, for example, 100% by mole or less with respect to all repeating units of the resin (A).

Examples of the repeating unit including at least any one of a fluorine atom, a bromine atom, or an iodine atom include a repeating unit having a fluorine atom, a bromine atom, or an iodine atom and having an acid-decomposable group, a repeating unit having a fluorine atom, a bromine atom, or an iodine atom and having an acid group, and a repeating unit having a fluorine atom, a bromine atom, or an iodine atom.

(Repeating Unit Having Lactone Group, Sultone Group, or Carbonate Group)

The resin (A) may have a repeating unit having at least one selected from the group consisting of a lactone group, a sultone group, and a carbonate group (hereinafter, also referred to as “unit Y”).

It is also preferable that the unit Y has no hydroxyl group and acid group such as a hexafluoroisopropanol group.

The lactone group or the sultone group may have a lactone structure or a sultone structure. The lactone structure or the sultone structure is preferably a 5- to 7-membered ring lactone structure or a 5- to 7-membered ring sultone structure. Among these, the structure is more preferably a 5- to 7-membered ring lactone structure with which another ring structure is fused so as to form a bicyclo structure or a spiro structure or a 5- to 7-membered ring sultone structure with which another ring structure is fused so as to form a bicyclo structure or a spiro structure.

The resin (A) preferably has a repeating unit having a lactone group or a sultone group, formed by extracting one or more hydrogen atoms from a ring member atom of a lactone structure represented by any of Formulae (LC1-1) to (LC1-21) or a sultone structure represented by any of Formulae (SL1-1) to (SL1-3).

In addition, the lactone group or the sultone group may be bonded directly to the main chain. For example, a ring member atom of the lactone group or the sultone group may constitute the main chain of the resin (A).

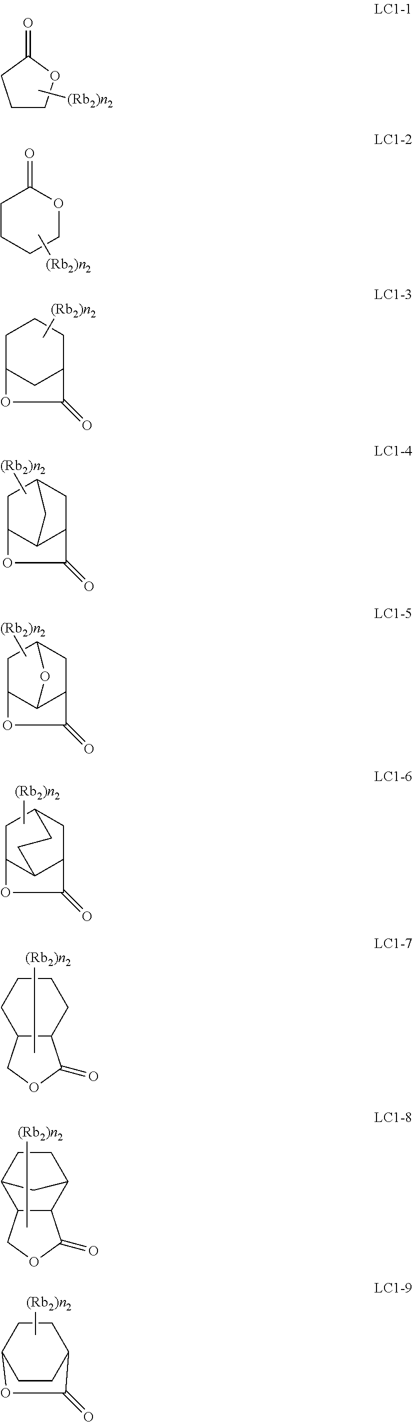

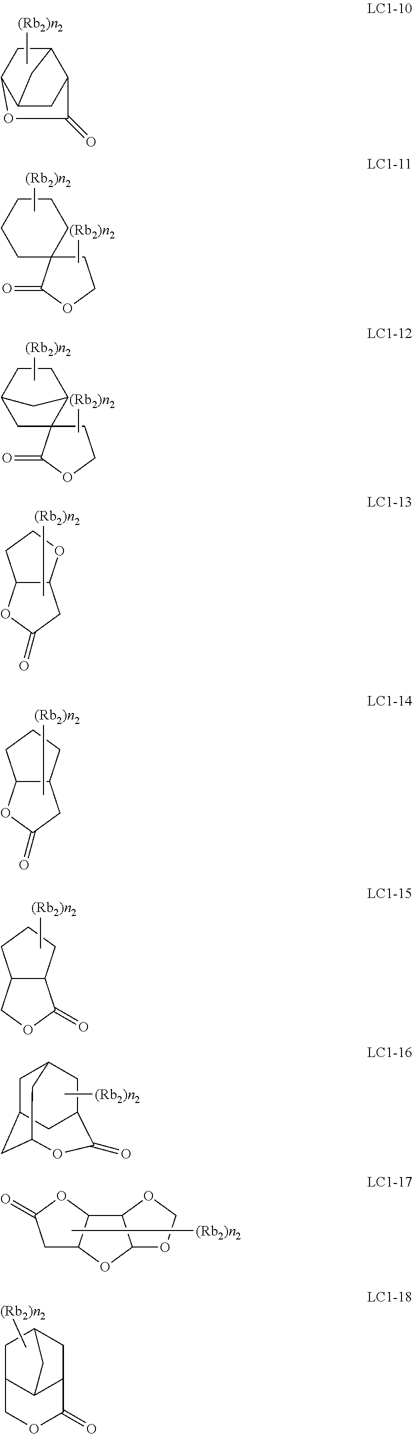

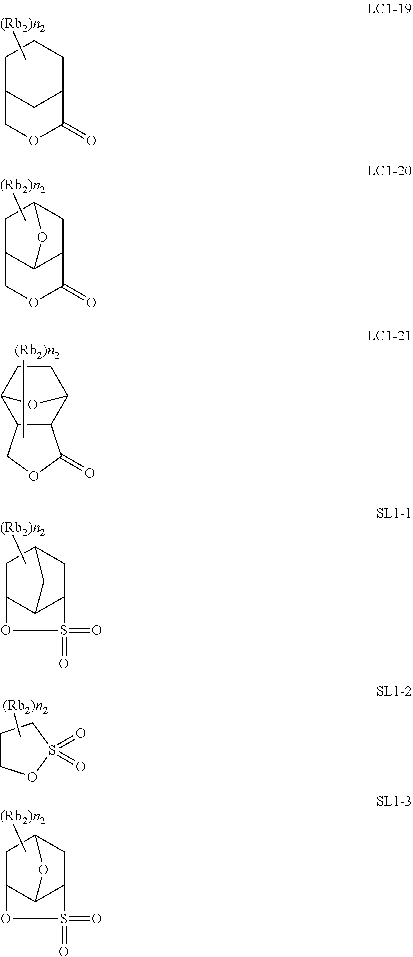

The above-described lactone structure or sultone structure may have a substituent (Rb₂). Preferred examples of the substituent (Rb₂) include an alkyl group having 1 to 8 carbon atoms, a cycloalkyl group having 4 to 7 carbon atoms, an alkoxy group having 1 to 8 carbon atoms, an alkoxycarbonyl group having 1 to 8 carbon atoms, a carboxyl group, a halogen atom, a cyano group, and an acid-decomposable group. n2 represents an integer of 0 to 4. In a case where n2 is 2 or more, a plurality of Rb₂'s may be different from each other, and the plurality of Rb₂'s may be bonded to each other to form a ring.

Examples of the repeating unit having a group including the lactone structure represented by any of Formulae (LC1-1) to (LC1-21) or the sultone structure represented by any of Formulae (SL1-1) to (SL1-3) include a repeating unit represented by Formula (AI).

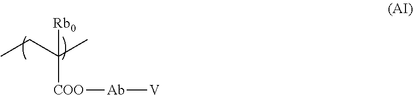

In Formula (AI), Rb₀ represents a hydrogen atom, a halogen atom, or an alkyl group having 1 to 4 carbon atoms. Preferred examples of the substituent which may be contained in the alkyl group of Rb₀ include a hydroxyl group and a halogen atom.

Examples of the halogen atom of Rb₀ include a fluorine atom, a chlorine atom, a bromine atom, and an iodine atom. Rb₀ is preferably a hydrogen atom or a methyl group.

Ab represents a single bond, an alkylene group, a divalent linking group having a monocyclic or polycyclic alicyclic hydrocarbon structure, an ether group, an ester group, a carbonyl group, a carboxyl group, or a divalent group formed by a combination thereof. Among these, as Ab, a single bond or a linking group represented by -Ab₁-CO₂— is preferable. Ab₁ is a linear or branched alkylene group, or a monocyclic or polycyclic cycloalkylene group, and is preferably a methylene group, an ethylene group, a cyclohexylene group, an adamantylene group, or a norbornylene group.

V represents a group formed by extracting one hydrogen atom from a ring member atom of the lactone structure represented by any of Formulae (LC1-1) to (LC1-21) or a group formed by extracting one hydrogen atom from a ring member atom of the sultone structure represented by any of Formulae (SL1-1) to (SL1-3).

In a case where an optical isomer is present in the repeating unit having a lactone group or a sultone group, any of optical isomers may be used. In addition, one optical isomer may be used alone or a mixture of a plurality of the optical isomers may be used. In a case where one kind of optical isomers is mainly used, an optical purity (ee) thereof is preferably 90 or more, and more preferably 95 or more.

As the carbonate group, a cyclic carbonate ester group is preferable.

As the repeating unit having a cyclic carbonate ester group, a repeating unit represented by Formula (A-1) is preferable.

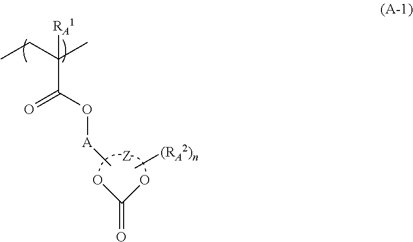

In Formula (A-1), R_{A}¹ represents a hydrogen atom, a halogen atom, or a monovalent organic group (preferably a methyl group). n represents an integer of 0 or more. R_{A}² represents a substituent. In a case where n is 2 or more, a plurality of R_{A}²'s may be the same or different from each other. A represents a single bond or a divalent linking group, As the above-described divalent linking group, an alkylene group, a divalent linking group having a monocyclic or polycyclic alicyclic hydrocarbon structure, an ether group, an ester group, a carbonyl group, a carboxyl group, or a divalent group formed by a combination thereof is preferable. Z represents an atomic group which forms a monocycle or polycycle with a group represented by —O—CO—O— in the formula.

The unit Y is shown below.

(in the formulae, Rx is H, CH₃, CH₂OH, or CF₃)

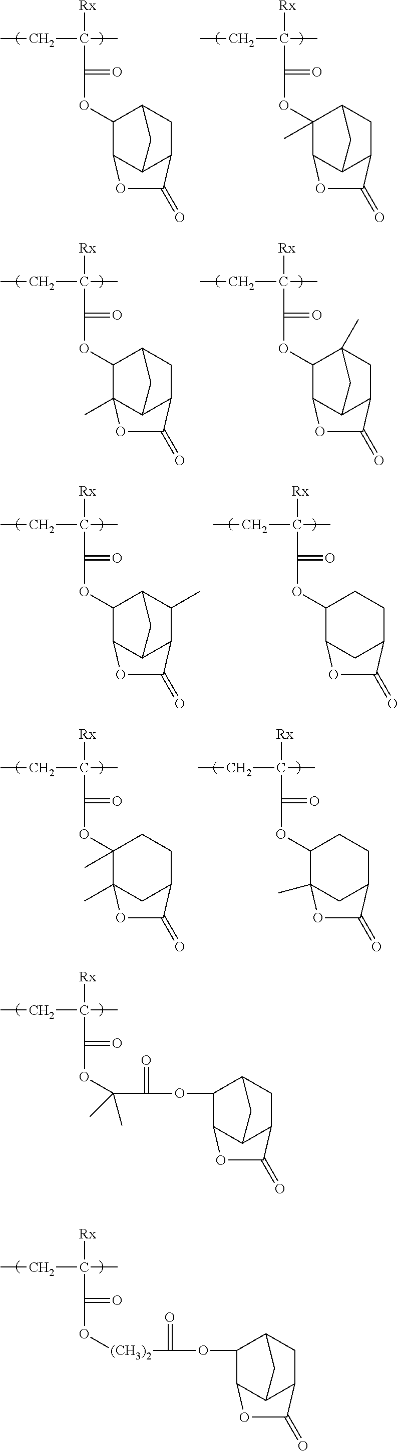

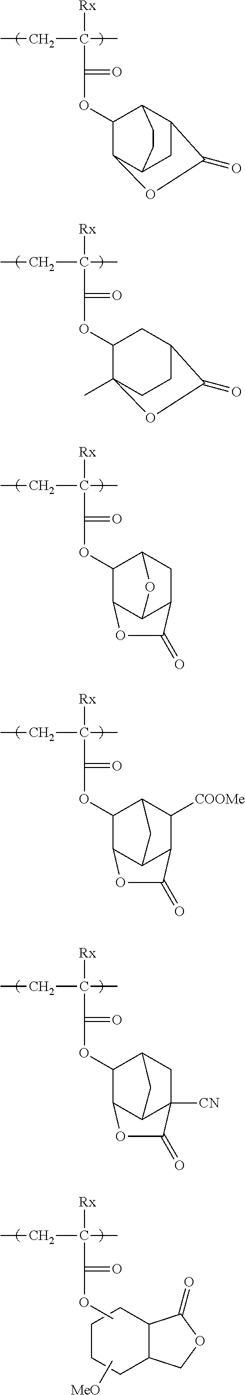

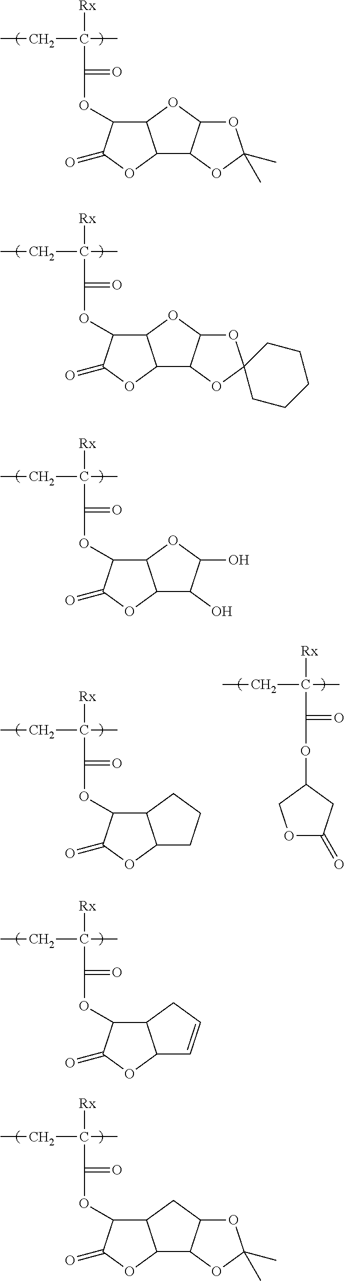

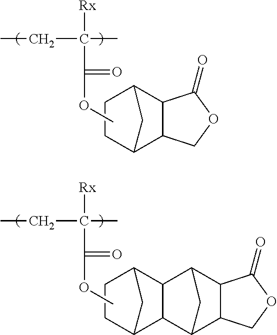

(in the formulae, Rx is H, CH₃, CH₂OH or CF₂)

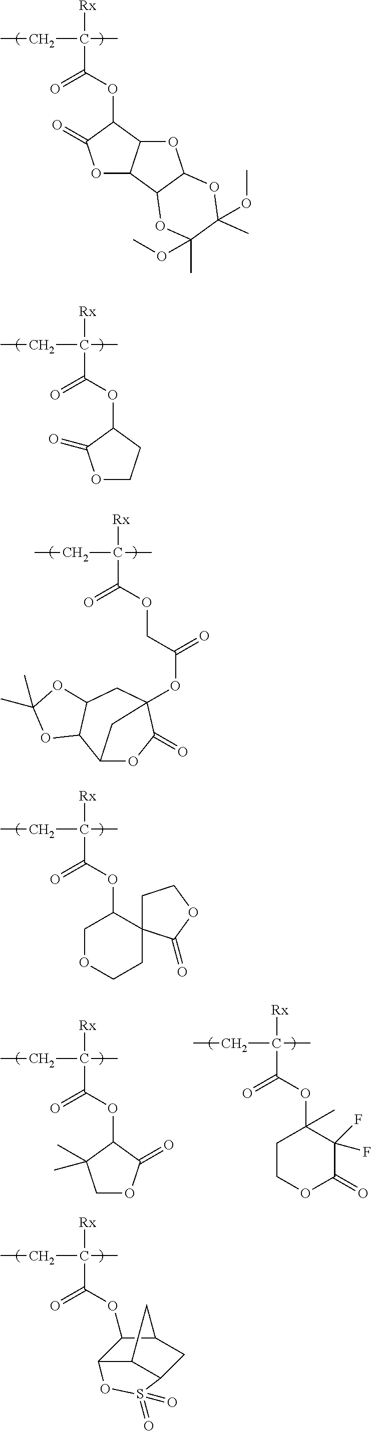

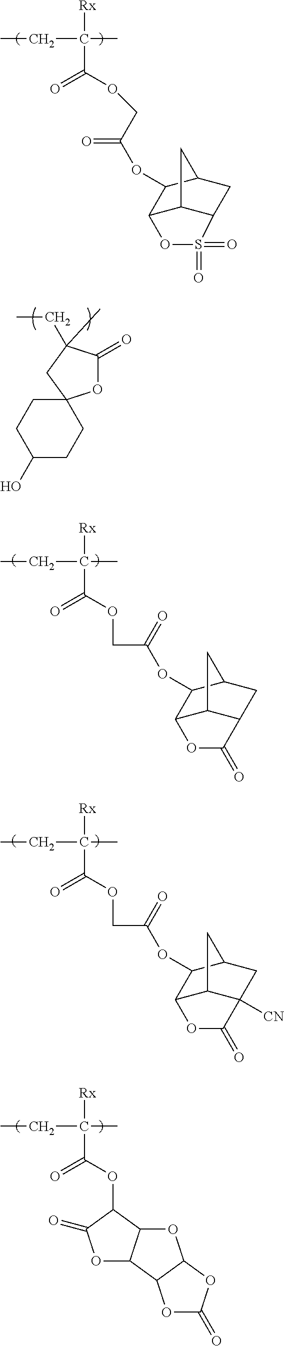

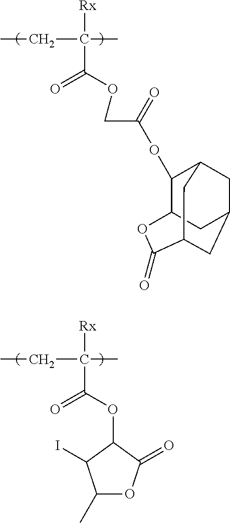

(in the formulae, Rx is H, CH₃, CH₂OH or CF₃)

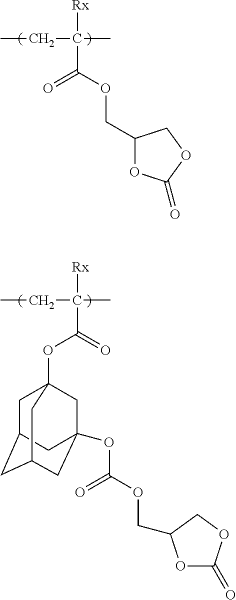

A content of the unit Y is preferably 1% by mole or more, and more preferably 10% by mole or more with respect to all repeating units in the resin (A). In addition, the upper limit value thereof is preferably 85% by mole or less, more preferably 80% by mole or less, still more preferably 70% by mole or less, and particularly preferably 60% by mole or less with respect to all repeating units in the resin (A).

(Repeating Unit Having Photoacid Generating Group)

The resin (A) may have, as a repeating unit other than those described above, a repeating unit having a group which generates an acid by irradiation with actinic ray or radiation (hereinafter, also referred to as a “photoacid generating group”).

Examples of the repeating unit having a photoacid generating group include a repeating unit represented by Formula (4).

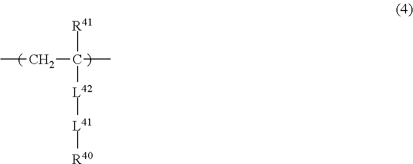

R⁴¹ represents a hydrogen atom or a methyl group. L⁴¹ represents a single bond or a divalent linking group. L⁴² represents a divalent linking group. R⁴⁰ represents a structural moiety which is decomposed by irradiation with actinic ray or radiation to generate an acid in a side chain.

The repeating unit having a photoacid generating group is exemplified below.

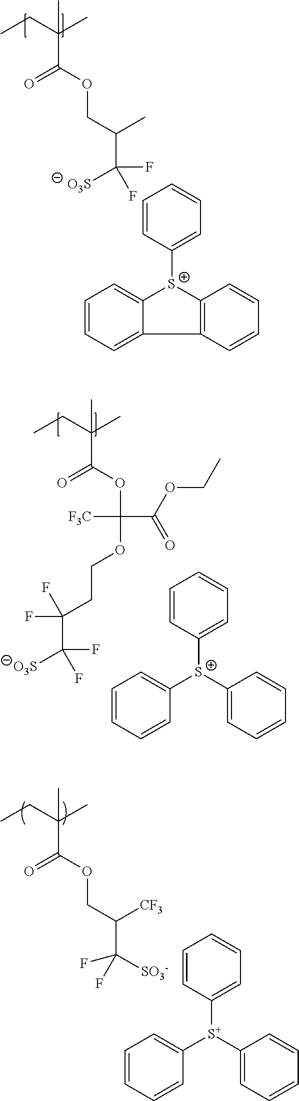

In addition, examples of the repeating unit represented by Formula (4) include repeating units described in paragraphs [0094] to [0105] of JP2014-041327A and repeating units described in paragraphs [0094] of WO2018/193954A.

A content of the repeating unit having a photoacid generating group is preferably 1% by mole or more, and more preferably 5% by mole or more with respect to all repeating units in the resin (A). In addition, the upper limit value thereof is preferably 40% by mole or less, more preferably 35% by mole or less, and still more preferably 30% by mole or less with respect to all repeating units in the resin (A).

(Repeating Unit Represented by Formula (V-1) or Formula (V-2))

The resin (A) may have a repeating unit represented by Formula (V-1) or Formula (V-2).

The repeating unit represented by Formula (V-1) and Formula (V-2) is preferably a repeating unit different from the above-described repeating units.

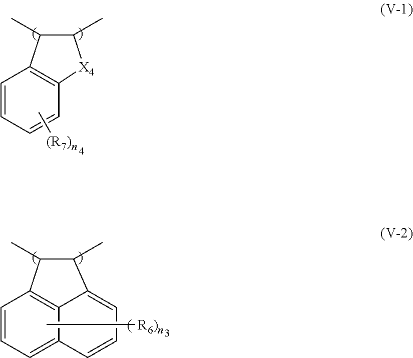

In the formulae,
R₆ and R₇ each independently represent a hydrogen atom, a hydroxyl group, an alkyl group, an alkoxy group, a cycloalkyl group, an acyloxy group, a cyano group, a nitro group, an amino group, a halogen atom, an ester group (—OCOR or —COOR: R is an alkyl group or fluorinated alkyl group having 1 to 6 carbon atoms), or a carboxyl group. As the alkyl group, a linear or branched alkyl group having 1 to 10 carbon atoms is preferable.

The cycloalkyl group may be a monocycle (cyclohexyl group or the like) or a polycycle (adamantyl group or the like), and the number of carbon atoms is preferably 3 to 15, more preferably 3 to 10, and still more preferably 3 to 6.

n₃ represents an integer of 0 to 6.

n₄ represents an integer of 0 to 4.

X⁴ is a methylene group, an oxygen atom, or a sulfur atom.

Examples of the repeating unit represented by Formula (V-1) or (V-2) include repeating units described in paragraph [0100] of WO2018/193954A.

(Repeating Unit for Reducing Mobility of Main Chain)

From the viewpoint that excessive diffusion of a generated acid or pattern collapse during development can be suppressed, the resin (A) preferably has a high glass transition temperature (Tg). The Tg is preferably higher than 90° C., more preferably higher than 100° C., still more preferably higher than 110° C., and particularly preferably higher than 125° C. From the viewpoint that a dissolution rate in the developer is excellent, the Tg is preferably 400° C. or lower and more preferably 350° C. or lower.

In the present specification, the glass transition temperature (Tg) of a polymer such as the resin (A) (hereinafter, “Tg of the repeating unit”) is calculated by the following method. First, each Tg of homopolymers consisting of only the respective repeating units included in the polymer is calculated by the Bicerano method. Next, the mass proportion (%) of each repeating unit to all repeating units in the polymer is calculated. Next, the Tg at each mass proportion is calculated using a Fox's equation (described in Materials Letters 62 (2008) 3152, and the like), and these are summed to obtain the Tg (° C.) of the polymer.

The Bicerano method is described in Prediction of polymer properties, Marcel Dekker Inc., New York (1993). In addition, the calculation of a Tg by the Bicerano method can be carried out using MDL Polymer (MDL Information Systems, Inc.), which is software for estimating physical properties of a polymer.

In order to raise the Tg of the resin (A) (preferably to raise the Tg to higher than 90° C.), it is preferable to reduce the mobility of the main chain of the resin (A). Examples of a method for lowering the mobility of the main chain of the resin (A) include the following (a) to (e) methods.
(a) introduction of a bulky substituent into the main chain
(b) introduction of a plurality of substituents into the main chain
(c) introduction of a substituent causing an interaction between the resins (A) into the vicinity of the main chain
(d) formation of the main chain in a cyclic structure
(e) linking of a cyclic structure to the main chain

The resin (A) preferably has a repeating unit in which the homopolymer exhibits a Tg of 130° C. or higher.

The type of the repeating unit in which the homopolymer exhibits a Tg of 130° C. or higher is not particularly limited, and may be any of repeating units in which the homopolymer exhibits a Tg of 130° C. or higher, as calculated by a Bicerano method. It corresponds to a repeating unit having a Tg of a homopolymer exhibiting 130° C. or higher, depending on the type of a functional group in the repeating units represented by Formulae (A) to (E), which will be described later.

As an example of a specific unit for accomplishing (a) above, a method of introducing a repeating unit represented by Formula (A) into the resin (A) may be mentioned.

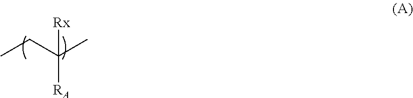

In Formula (A), R_{A} represents a group including a polycyclic structure. Rx represents a hydrogen atom, a methyl group, or an ethyl group. The group including a polycyclic structure is a group including a plurality of ring structures, and the plurality of ring structures may or may not be fused.

Specific examples of the repeating unit represented by Formula (A) include repeating units described in paragraphs [0107] to [0119] of WO2018/193954A.

As an example of a specific unit for accomplishing (b) above, a method of introducing a repeating unit represented by Formula (B) into the resin (A) may be mentioned.

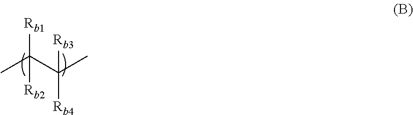

In Formula (B), R_{b1} to R_{b4} each independently represent a hydrogen atom or an organic group, and at least two or more of R_{b1}, . . . , or R_{b4} represent an organic group.

In addition, in a case where at least one of the organic groups is a group in which a ring structure is directly linked to the main chain in the repeating unit, the types of the other organic groups are not particularly limited.

In addition, in a case where none of the organic groups is a group in which a ring structure is directly linked to the main chain in the repeating unit, at least two or more of the organic groups are substituents having three or more constituent atoms excluding hydrogen atoms.

Specific examples of the repeating unit represented by Formula (B) include repeating units described in paragraphs [0113] to [0115] of WO2018/193954A.

As an example of a specific unit for accomplishing (c) above, a method of introducing a repeating unit represented by Formula (C) into the resin (A) may be mentioned.

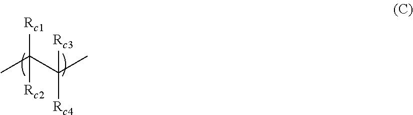

In Formula (C), R_{c1} to R_{c4} each independently represent a hydrogen atom or an organic group, and at least one of R_{c1}, . . . , or R_{c4} is a group including a hydrogen-bonding hydrogen atom with the number of atoms of 3 or less from the main chain carbon. Among these, it is preferable that the group has hydrogen-bonding hydrogen atoms with the number of atoms of 2 or less (on a side closer to the vicinity of the main chain) to cause an interaction between the main chains of the resin (A).

Specific examples of the repeating unit represented by Formula (C) include repeating units described in paragraphs [0119] to [0121] of WO2018/193954A.

As an example of a specific unit for accomplishing (d) above, a method of introducing a repeating unit represented by Formula (D) into the resin (A) may be mentioned.

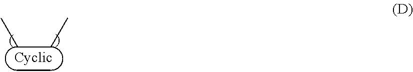

In Formula (D), “Cyclic” is a group which forms a main chain as a cyclic structure. The number of ring-constituting atoms is not particularly limited.

Specific examples of the repeating unit represented by Formula (D) include repeating units described in paragraphs [0126] and [0127] of WO2018/193954A.

As an example of a specific unit for accomplishing (e) above, a method of introducing a repeating unit represented by Formula (E) into the resin (A) may be mentioned.

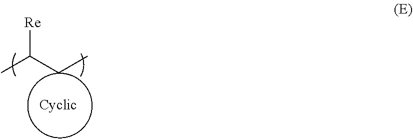

In Formula (E), Re's each independently represent a hydrogen atom or an organic group. Examples of the organic group include an alkyl group, a cycloalkyl group, an aryl group, an aralkyl group, and an alkenyl group, which may have a substituent.

“Cyclic” is a cyclic group including a carbon atom of a main chain. The number of atoms included in the cyclic group is not particularly limited.

Specific examples of the repeating unit represented by Formula (E) include repeating units described in paragraphs [0131] to [0133] of WO2018/193954A.

(Repeating Unit Having at Least One Group Selected from Lactone Group, Sultone Group, Carbonate Group, Hydroxyl Group, Cyano Group, or Alkali-Soluble Group)

The resin (A) may have a repeating unit having at least one group selected from a lactone group, a sultone group, a carbonate group, a hydroxyl group, a cyano group, or an alkali-soluble group.

Examples of the repeating unit having a lactone group, a sultone group, or a carbonate group included in the resin (A) include the repeating units described in <repeating unit having lactone group, sultone group, or carbonate group> described above. A preferred content thereof is also the same as described in <repeating unit having lactone Group, sultone Group, or carbonate group> mentioned above.

The resin (A) may have a repeating unit having a hydroxyl group or a cyano group. As a result, adhesiveness to the substrate and affinity for a developer are improved.

The repeating unit having a hydroxyl group or a cyano group is preferably a repeating unit having an alicyclic hydrocarbon structure substituted with a hydroxyl group or a cyano group.

It is preferable that the repeating unit having a hydroxyl group or a cyano group does not have the acid-decomposable group. Examples of the repeating unit having a hydroxyl group or a cyano group include repeating units described in paragraphs [0081] to [0084] of JP2014-098921A.

As a preferred aspect, examples of the hydroxyl group include an alcoholic hydroxyl group.

In addition, in a case where the above-described resin (A) has a repeating unit having an alcoholic hydroxyl group, a content of the repeating unit having an alcoholic hydroxyl group is preferably 5% by mole or more, and more preferably 10% by mole or more with respect to all repeating units in the resin (A). In addition, the upper limit value thereof is preferably 70% by mole or less, more preferably 60% by mole or less, and still more preferably 50% by mole or less with respect to all repeating units in the resin (A).

The resin (A) may have a repeating unit having an alkali-soluble group.

Examples of the alkali-soluble group include a carboxyl group, a sulfonamide group, a sulfonylimide group, a bissulfonylimide group, and an aliphatic alcohol group (for example, a hexafluoroisopropanol group) in which the α-position is substituted with an electron withdrawing group, and a carboxyl group is preferable. In a case where the resin (A) includes the repeating unit having an alkali-soluble group, resolution for use in contact holes is increased. Examples of the repeating unit having an alkali-soluble group include repeating units described in paragraphs [0085] and [0086] of JP2014-098921A.

(Repeating Unit Having Alicyclic Hydrocarbon Structure and not Exhibiting Acid Decomposability)

The resin (A) may have a repeating unit having an alicyclic hydrocarbon structure and not exhibiting acid decomposability. As a result, it is possible to reduce elution of low-molecular-weight components from the resist film into the immersion liquid during liquid immersion exposure. Examples of such a repeating unit include a repeating unit derived from 1-adamantyl (meth)acrylate, diamantyl (meth)acrylate, tricyclodecanyl (meth)acrylate, or cyclohexyl (meth)acrylate.

(Repeating Unit Represented by Formula (III) Having Neither Hydroxyl Group Nor Cyano Group)

The resin (A) may have a repeating unit represented by Formula (III), which has neither a hydroxyl group nor a cyano group.

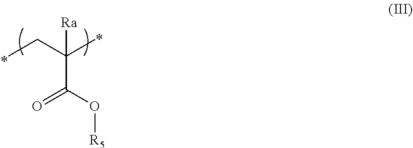

In Formula (III), R₅ represents a hydrocarbon group having at least one cyclic structure and having neither a hydroxyl group nor a cyano group.

Ra represents a hydrogen atom, an alkyl group, or a —CH₂—O—Ra₂ group. In the formula, Ra₂ represents a hydrogen atom, an alkyl group, or an acyl group.

Examples of the repeating unit represented by Formula (III), which has neither a hydroxyl group nor a cyano group, include repeating units described in paragraphs [0087] to

of JP2014-098921A.

(Other Repeating Units)

Furthermore, the resin (A) may have a repeating unit other than the above-described repeating units.

For example, the resin (A) may have a repeating unit selected from the group consisting of a repeating unit having an oxathiane ring group, a repeating unit having an oxazolone ring group, a repeating unit having a dioxane ring group, and a repeating unit having a hydantoin ring group.

Such repeating units are exemplified below.

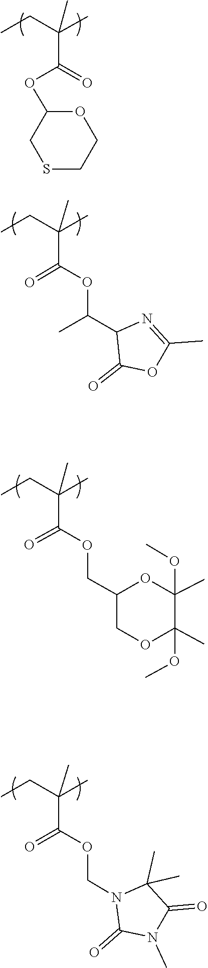

For the purpose of controlling dry etching resistance, suitability for a standard developer, substrate adhesiveness, resist profile, resolution, heat resistance, sensitivity, and the like, the resin (A) may have various repeating units in addition to the repeating units described above.

As the resin (A), (in particular, in a case where the composition is used as an actinic ray-sensitive or radiation-sensitive resin composition with ArF), it is preferable that all repeating units are composed of repeating units derived from a compound having an ethylenically unsaturated bond. In particular, it is also preferable that all repeating units are composed of (meth)acrylate-based repeating units. In this case, any resin of a resin in which all repeating units are methacrylate-based repeating units, a resin in which all repeating units are acrylate-based repeating units, or a resin with all repeating units consisting of a methacrylate-based repeating unit and an acrylate-based repeating unit can be used. In addition, the acrylate-based repeating unit is preferably 50% by mole or less of all repeating units.

The resin (A) can be synthesized in accordance with an ordinary method (for example, a radical polymerization).

A weight-average molecular weight of the resin (A) as a value expressed in terms of polystyrene by a GPC method is preferably 30,000 or less, more preferably 1,000 to 30,000, still more preferably 3,000 to 30,000, and particularly preferably 5,000 to 15,000.

A dispersity (molecular weight distribution) of the resin (A) is preferably 1 to 5, more preferably 1 to 3, still more preferably 1.2 to 3.0, and particularly preferably 1.2 to 2.0. As the dispersity is smaller, resolution and resist shape are more excellent, and a side wall of a resist pattern is smoother and roughness is also more excellent.

In the composition according to the embodiment of the present invention, a content of the resin (A) is preferably 30.0% to 99.9% by mass, more preferably 60.0% to 90.0% by mass, and still more preferably 60.0% to 85.0% by mass with respect to the total solid content of the composition according to the embodiment of the present invention.

The resin (A) may be used alone or in combination of two or more kinds thereof.

As long as effects of the present invention are not impaired, the composition according to the embodiment of the present invention may contain a resin (also referred to as a resin (A′)) different from the resin (A), in addition to the resin (A).

The resin (A′) is not particularly limited as long as it is a resin different from the resin (A), and examples thereof include a resin which, in the resin (A), does not have an acid group, an alcoholic hydroxyl group, or an acid-decomposable group.

In a case where the composition according to the embodiment of the present invention contains the resin (A′), a ratio of the content of the resin (A) and the content of the resin (A′) in the composition according to the embodiment of the present invention is preferably 9:1 to 8:2 in a mass ratio.

<(B) Compound which Generates Acid by Irradiation with Actinic Ray or Radiation>

The composition according to the embodiment of the present invention contains a compound which generates an acid by irradiation with actinic ray or radiation (also referred to as a compound (B), a photoacid generator, or a photoacid generator (B)). The photoacid generator is a compound which generates an acid by exposure.

The photoacid generator (B) may be in a form of a low-molecular-weight compound or a form incorporated into a part of a polymer (for example, the above-described resin (A)). In addition, a combination of the form of a low-molecular-weight compound and the form incorporated into a part of a polymer (for example, the above-described resin (A)) may also be used.

In a case where the photoacid generator (B) is in the form of a low-molecular-weight compound, a molecular weight of the photoacid generator is preferably 3,000 or less, more preferably 2,000 or less, and still more preferably 1,000 or less. The upper limit thereof is not particularly limited, and is preferably 100 or more.

In a case where the photoacid generator (B) is in the form incorporated into a part of a polymer, it may be incorporated into the part of the resin (A) or into a resin which is different from the resin (A).

In the present invention, the photoacid generator (B) is preferably in the form of a low-molecular-weight compound.

The photoacid generator (B) may be an ionic compound having a cation and an anion.

As a preferred aspect, it is preferable that the above-described compound (B) is an ionic compound having a reactive moiety (2) in an anionic moiety. The above-described reactive moiety (2) is not particularly limited, and examples thereof include a partial structure represented by any of General Formula (1), (2), or (3).

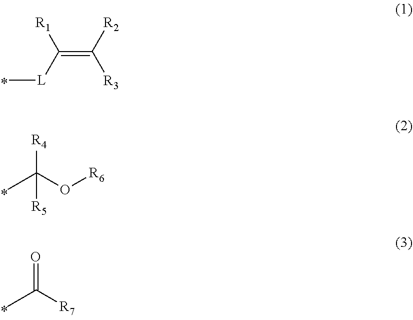

In General Formula (1), R₁ to R₃ each independently represent a hydrogen atom or a substituent, L represents a single bond or a divalent linking group, and * represents a bonding position.

In General Formula (2), R₄ to R₆ each independently represent a hydrogen atom or a substituent, and * represents a bonding position.

In General Formula (3), R₇ represents a hydrogen atom or a substituent, and * represents a bonding position.

The substituent of R₁ to R₃ is not particularly limited, and examples thereof include an alkyl group.

The alkyl group is not particularly limited, examples thereof include a linear or branched alkyl group having 1 to 12 carbon atoms, and an alkyl group having 1 to 6 carbon atoms is preferable and an alkyl group having 1 to 3 carbon atoms is more preferable.

Examples of the divalent linking group of L include —COO—, —CO—, —O—, an alkylene group, a cycloalkylene group, an arylene group, and a linking group in which a plurality of these groups are linked.

The alkylene group is not particularly limited, and may be linear or branched. The number of carbon atoms in the alkylene group is not particularly limited, but is preferably 1 to 10 and more preferably 1 to 3.

The cycloalkylene group is not particularly limited, and may be monocyclic or polycyclic. The number of carbon atoms in the cycloalkylene group is not particularly limited, but is preferably 3 to 10 and more preferably 3 to 6.

The arylene group is not particularly limited, and an arylene group having 6 to 14 carbon atoms is preferable and an arylene group having 6 to 10 carbon atoms is more preferable.

The alkylene group, cycloalkylene group, and arylene group may have a substituent.

As a preferred aspect, the substituent is not particularly limited, and examples thereof include an alkyl group and a halogen atom. The alkyl group is not particularly limited, examples thereof include a linear or branched alkyl group having 1 to 12 carbon atoms, and an alkyl group having 1 to 6 carbon atoms is preferable and an alkyl group having 1 to 3 carbon atoms is more preferable. Examples of the halogen atom include a fluorine atom, a chlorine atom, a bromine atom, and an iodine atom.

L is preferably a single bond, —COO—, or —O—.

R₂ and R₃ preferably represent a hydrogen atom.

The substituent of R₄ to R₆ is not particularly limited, and examples thereof include an alkyl group.

The alkyl group is not particularly limited, examples thereof include a linear or branched alkyl group having 1 to 12 carbon atoms, and an alkyl group having 1 to 6 carbon atoms is preferable and an alkyl group having 1 to 3 carbon atoms is more preferable.

R₆ preferably represents a hydrogen atom.

The substituent of R₇ is not particularly limited, and examples thereof include an alkyl group.

The alkyl group is not particularly limited, examples thereof include a linear or branched alkyl group having 1 to 12 carbon atoms, and an alkyl group having 1 to 6 carbon atoms is preferable and an alkyl group having 1 to 3 carbon atoms is more preferable.

The above-described partial structure is preferably represented by General Formula (1) or General Formula (3) described above.

The above-described partial structure is preferably a partial structure selected from the following structures.

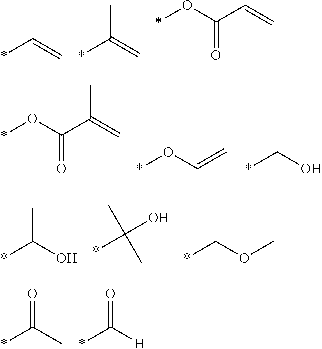

* represents a bonding position.

The above-described partial structure is preferably a partial structure selected from the following structures.

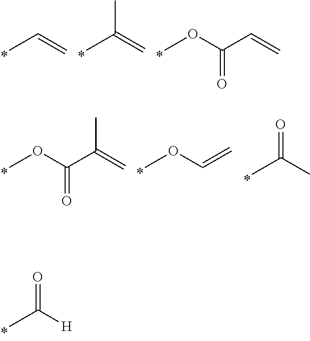

* represents a bonding position.

The reactive species generated from the above-described reactive moiety (2) is not particularly limited, and examples thereof include a moiety which is to be a carbocation in a case where the acid generated from the compound (B) attacks another compound (B) at the above-described partial structure represented by any of General Formula (1), (2), or (3).

In addition, examples of the reactive species also include a carbon atom charged with δ⁺, which is not to be the carbocation.

As a preferred aspect, the above-described compound (B) is an ionic compound having a partial structure represented by any of General Formula (1), (2), or (3) in an anionic moiety.

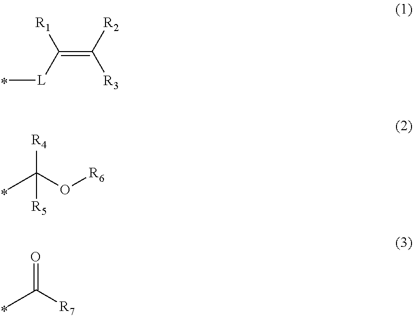

In Formula (1), R₁ to R₃ each independently represent a hydrogen atom or a substituent, L represents a single bond or a divalent linking group, and * represents a bonding position.

In Formula (2), R₄ to R₆ each independently represent a hydrogen atom or a substituent, and * represents a bonding position.

In Formula (3), R₇ represents a hydrogen atom or a substituent, and * represents a bonding position.

Each group in General Formulae (1) to (3) is as described above.

Each of the partial structures represented by any of General Formula (1), (2), or (3) can be the reactive moiety (2). The reactive moiety (2) may be a moiety where a reactive species is generated by actinic ray or radiation or by action of acid.

The above-described partial structure is preferably represented by General Formula (1) or General Formula (3) described above.

The above-described partial structure is preferably a partial structure selected from the following structures.

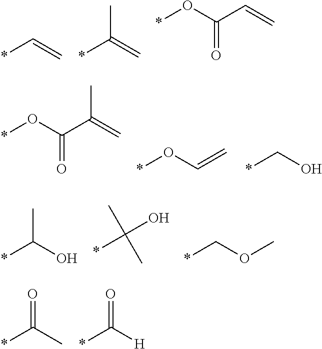

* represents a bonding position.

The above-described partial structure is preferably a partial structure selected from the following structures.

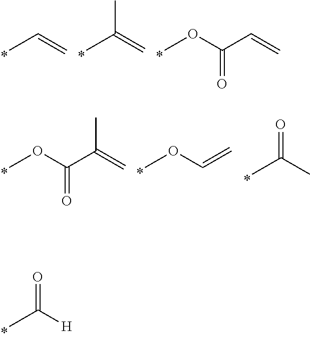

* represents a bonding position.

The above-described compound (B) may or may not have a partial structure represented by any of General Formula (11), . . . , (14) in the anionic moiety, but since the polymerization reaction of the above-described compound (B) tends to proceed, it is preferable that the above-described compound (B) does not have a partial structure represented by any of General Formula (11), . . . , (14) in the anionic moiety.

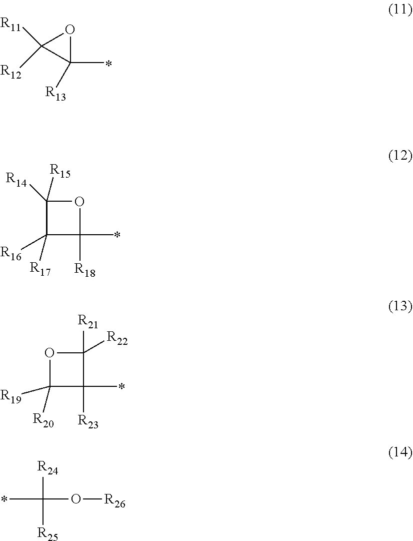

In General Formula (11), R₁ to R₁₃ each independently represent a hydrogen atom or a substituent, and * represents a bonding position.

In General Formula (12), R₁₄ to R₁₅ each independently represent a hydrogen atom or a substituent, and * represents a bonding position.

In General Formula (13), R₁₉ to R₂₃ each independently represent a hydrogen atom or a substituent, and * represents a bonding position.

In General Formula (14), R₂₄ to R₂₆ each independently represent a hydrogen atom or a substituent, and * represents a bonding position.

The substituent of R₁₁ to R₁₃ is not particularly limited as long as it is a monovalent substituent, and examples thereof include the above-described substituent T.

The substituent of R₁₄ to R₁₅ is not particularly limited as long as it is a monovalent substituent, and examples thereof include the above-described substituent T.

The substituent of R₁₉ to R₂₃ is not particularly limited as long as it is a monovalent substituent, and examples thereof include the above-described substituent T.

The substituent of R₂₄ to R₂₆ is not particularly limited as long as it is a monovalent substituent, and examples thereof include the above-described substituent T.

It is preferable that the acid generated from the above-described compound (B) includes an aromatic ring. The aromatic ring is not particularly limited, and may be a monocycle or a polycycle. Examples of the aromatic ring include a benzene ring, a naphthalene ring, and an anthracene ring.

Examples of the photoacid generator (B) include a compound represented by “M⁺X⁻” (onium salt), and a compound which generates an organic acid by exposure is preferable.

Examples of the above-described organic acid include sulfonic acids (an aliphatic sulfonic acid, an aromatic sulfonic acid, a camphor sulfonic acid, and the like), carboxylic acids (an aliphatic carboxylic acid, an aromatic carboxylic acid, an aralkylcarboxylic acid, and the like), a carbonylsulfonylimide acid, a bis(alkylsulfonyl)imide acid, and a tris(alkylsulfonyl)methide acid.

In the compound represented by “M⁺X⁻”, M⁺ represents an organic cation.

The organic cation is not particularly limited. In addition, a valence of the organic cation may be 1 or 2 or more.

Among these, as the above-described organic cation, a cation represented by Formula (ZaI) (hereinafter, also referred to as “cation (ZaI)”) or a cation represented by Formula (ZaII) (hereinafter, also referred to as “cation (ZaII)”) is preferable.

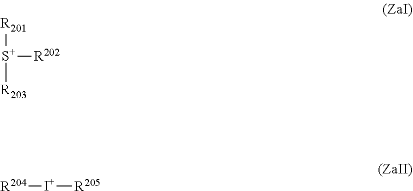

In Formula (ZaI),
R²⁰¹, R²⁰², and R²⁰³ each independently represent an organic group.

The number of carbon atoms in the organic group of R²⁰¹, R²⁰², and R²⁰³ is preferably 1 to 30 and more preferably 1 to 20. In addition, two of R²⁰¹ to R²⁰³ may be bonded to each other to form a ring structure, and the ring structure may include an oxygen atom, a sulfur atom, an ester group, an amide group, or a carbonyl group in the ring. Examples of the group formed by the bonding of two of R²⁰¹ to R²⁰³ include an alkylene group (for example, a butylene group and a pentylene group) and —CH₂—CH₂—O—CH₂—CH₂—.

Examples of suitable aspects of the organic cation in Formula (ZaI) include a cation (ZaI-1), a cation (ZaI-2), an organic cation (cation (ZaI-3b)) represented by Formula (ZaI-3b), and an organic cation (cation (ZaI-4b)) represented by Formula (ZaI-4b), each of which will be described later.

First, the cation (ZaI-1) will be described.

The cation (ZaI-1) is an arylsulfonium cation in which at least one of R²⁰¹, R²⁰², or R²⁰³ of Formula (ZaI) described above is an aryl group.

In the arylsulfonium cation, all of R²⁰¹ to R²⁰³ may be aryl groups, or some of R²⁰¹ to R²⁰³ may be an aryl group and the rest may be an alkyl group or a cycloalkyl group.

In addition, one of R²⁰¹ to R²⁰³ may be an aryl group, the remaining two of R²⁰¹ to R²¹³ may be bonded to each other to form a ring structure, and an oxygen atom, a sulfur atom, an ester group, an amide group, or a carbonyl group may be included in the ring. Examples of the group formed by the bonding of two of R²⁰¹ to R²⁰³ include an alkylene group (for example, a butylene group, a pentylene group, and —CH₂—CH₂—O—CH₂—CH₂—) in which one or more methylene groups may be substituted with an oxygen atom, a sulfur atom, an ester group, an amide group, and/or a carbonyl group.

Examples of the arylsulfonium cation include a triarylsulfonium cation, a diarylalkylsulfonium cation, an aryldialkylsulfonium cation, a diarylcycloalkylsulfonium cation, and an aryldicycloalkylsulfonium cation.

The aryl group included in the arylsulfonium cation is preferably a phenyl group or a naphthyl group, and more preferably a phenyl group. The aryl group may be an aryl group which has a heterocyclic structure having an oxygen atom, a nitrogen atom, a sulfur atom, or the like. Examples of the heterocyclic structure include a pyrrole residue, a furan residue, a thiophene residue, an indole residue, a benzofuran residue, and a benzothiophene residue. In a case where the arylsulfonium cation has two or more aryl groups, the two or more aryl groups may be the same or different from each other.

The alkyl group or the cycloalkyl group included in the arylsulfonium cation as necessary is preferably a linear alkyl group having 1 to 15 carbon atoms, a branched alkyl group having 3 to 15 carbon atoms, or a cycloalkyl group having 3 to 15 carbon atoms, and more preferably a methyl group, an ethyl group, a propyl group, an n-butyl group, a sec-butyl group, a t-butyl group, a cyclopropyl group, a cyclobutyl group, or a cyclohexyl group.

The substituent which may be contained in the aryl group, alkyl group, and cycloalkyl group of R²⁰¹ to R²⁰³ is preferably an alkyl group (for example, having 1 to 15 carbon atoms), a cycloalkyl group (for example, having 3 to 15 carbon atoms), an aryl group (for example, having 6 to 14 carbon atoms), an alkoxy group (for example, having 1 to 15 carbon atoms), a cycloalkyl alkoxy group (for example, having 1 to 15 carbon atoms), a halogen atom (for example, fluorine and iodine), a hydroxyl group, a carboxyl group, an ester group, a sulfinyl group, a sulfonyl group, an alkylthio group, or a phenylthio group.

The substituent may further have a substituent if possible, and it is also preferable that the above-described alkyl group has a halogen atom as the substituent to form an alkyl halide group such as a trifluoromethyl group.

In addition, it is also preferable to form an acid-decomposable group by any combination of the above-described substituents.

The acid-decomposable group is intended to a group which is decomposed by action of acid to form a polar group, and preferably has a structure in which a polar group is protected by a leaving group which is eliminated by action of acid. The above-described polar group and leaving group are as described above.

Next, the cation (ZaI-2) will be described.

The cation (ZaI-2) is a cation in which R²⁰¹ to R²⁰³ in Formula (ZaI) are each independently a cation representing an organic group having no aromatic ring. The aromatic ring also encompasses an aromatic ring including a heteroatom.

The number of carbon atoms in the organic group as R²⁰¹ to R²⁰³, which has no aromatic ring, is preferably 1 to 30, and more preferably 1 to 20.

R²⁰¹ to R²⁰³ are each independently preferably an alkyl group, a cycloalkyl group, an allyl group, or a vinyl group, more preferably a linear or branched 2-oxoalkyl group, a 2-oxocycloalkyl group, or an alkoxycarbonylmethyl group, and still more preferably a linear or branched 2-oxoalkyl group.

Examples of the alkyl group and cycloalkyl group of R²⁰¹ to R²⁰³ include a linear alkyl group having 1 to 10 carbon atoms or a branched alkyl group having 3 to 10 carbon atoms (for example, a methyl group, an ethyl group, a propyl group, a butyl group, and a pentyl group), and a cycloalkyl group having 3 to 10 carbon atoms (for example, a cyclopentyl group, a cyclohexyl group, and a norbornyl group).

R²⁰¹ to R²⁰³ may further be substituted with a halogen atom, an alkoxy group (for example, having 1 to 5 carbon atoms), a hydroxyl group, a cyano group, or a nitro group.

In addition, it is also preferable that the substituents of R²⁰¹ to R²⁰³ each independently form an acid-decomposable group by any combination of the substituents.

Next, the cation (ZaI-3b) will be described.

The cation (ZaI-3b) is a cation represented by Formula (ZaI-3b).

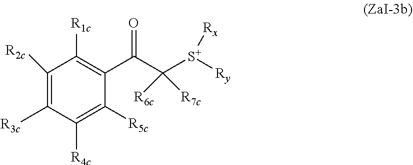

In General Formula (ZaI-3b),
R_{1c} to R_{5c} each independently represent a hydrogen atom, an alkyl group, a cycloalkyl group, an aryl group, an alkoxy group, an aryloxy group, an alkoxycarbonyl group, an alkylcarbonyloxy group, a cycloalkylcarbonyloxy group, a halogen atom, a hydroxyl group, a nitro group, an alkylthio group, or an arylthio group,
R_{6c} and R_{7c} each independently represent a hydrogen atom, an alkyl group (for example, a t-butyl group and the like), a cycloalkyl group, a halogen atom, a cyano group, or an aryl group, and
Rₓ and R_{y} each independently represent an alkyl group, a cycloalkyl group, a 2-oxoalkyl group, a 2-oxocycloalkyl group, an alkoxycarbonylalkyl group, an allyl group, or a vinyl group.

In addition, it is also preferable that the substituents of R_{1c} to R_{7c}, Rₓ, and R_{y} each independently form an acid-decomposable group by any combination of the substituents.

Any two or more of R_{1c}, . . . , or R_{5c}, R_{5c} and R_{6c}, R_{6c} and R_{7c}, R_{5c} and Rₓ, and Rₓ and R_{y} may each be bonded to each other to form a ring, and the rings may each independently include an oxygen atom, a sulfur atom, a ketone group, an ester bond, or an amide bond.

Examples of the above-described ring include an aromatic or non-aromatic hydrocarbon ring, an aromatic or non-aromatic heterocyclic ring, and a polycyclic fused ring formed by a combination of two or more of these rings. Examples of the ring include a 3- to 10-membered ring, and the ring is preferably a 4- to 8-membered ring and more preferably a 5- or 6-membered ring.

Examples of the group formed by the bonding of any two or more of R_{1c}, . . . , or R_{5c}, R_{6c} and R_{7c}, and Rₓ and R_{y} include an alkylene group such as a butylene group and a pentylene group. A methylene group in this alkylene group may be substituted with a heteroatom such as an oxygen atom.

As the group formed by the bonding of R_{5c} and R_{6c}, and R_{5c} and Rₓ, a single bond or an alkylene group is preferable. Examples of the alkylene group include a methylene group and an ethylene group.

The ring formed by bonding R_{1c} to R_{5c}, R_{6c}, R_{7c}, Rₓ, R_{y}, any two or more of R_{1c}, . . . , or R_{5c}, R_{5c} and R_{6c}, R_{6c} and R_{7c}, R_{5c} and Rₓ, and Rₓ and R_{y} to each other may have a substituent.

Next, the cation (ZaI-4b) will be described.

The cation (ZaI-4b) is a cation represented by Formula (ZaI-4b).

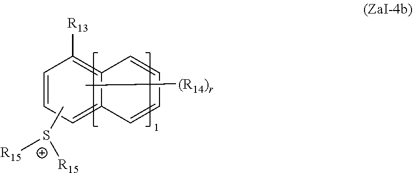

In Formula (ZaI-4b),
l represents an integer of 0 to 2.
r represents an integer of 0 to 8.
R₁₃ represents a hydrogen atom, a halogen atom (for example, a fluorine atom, an iodine atom, or the like), a hydroxyl group, an alkyl group, an alkyl halide group, an alkoxy group, a carboxyl group, an alkoxycarbonyl group, or a group having a cycloalkyl group (which may be the cycloalkyl group itself or a group including the cycloalkyl group in a part thereof). These groups may have a substituent.
R₁₄ represents a hydroxyl group, a halogen atom (for example, a fluorine atom, an iodine atom, or the like), an alkyl group, an alkyl halide group, an alkoxy group, an alkoxycarbonyl group, an alkylcarbonyl group, an alkylsulfonyl group, a cycloalkylsulfonyl group, or a group including a cycloalkyl group (which may be the cycloalkyl group itself or a group including the cycloalkyl group in a part thereof). These groups may have a substituent. In a case of a plurality of R₁₄'s, R₁₄'s each independently represent the above-described group such as a hydroxyl group.
R₁₅'s each independently represent an alkyl group, a cycloalkyl group, or a naphthyl group. Two R₁₅'s may be bonded to each other to form a ring. In a case where two R₁₅'s are bonded to each other to form a ring, the ring skeleton may include a heteroatom such as an oxygen atom and a nitrogen atom.

In one aspect, it is preferable that two R₁₅'s are alkylene groups and are bonded to each other to form a ring structure. The above-described alkyl group, the above-described cycloalkyl group, the above-descried naphthyl group, and the ring formed by bonding two R₁₅'s to each other may have a substituent.

In Formula (ZaI-4b), the alkyl group of R₁₃, R₁₄, and R₁₅ may be linear or branched. The number of carbon atoms in the alkyl group is preferably 1 to 10. The alkyl group is preferably a methyl group, an ethyl group, an n-butyl group, a t-butyl group, or the like.

In addition, it is also preferable that the substituents of R₁₃ to R₁₅, Rₓ, and R_{y} each independently form an acid-decomposable group by any combination of the substituents.

Next, Formula (ZaII) will be described.

In Formula (ZaII), R²⁰⁴ and R²⁰⁵ each independently represent an aryl group, an alkyl group, or a cycloalkyl group.

The aryl group of R²⁰⁴ and R²⁰⁵ is preferably a phenyl group or a naphthyl group, and more preferably a phenyl group. The aryl group of R²⁰⁴ and R²⁰⁵ may be an aryl group which has a heterocyclic ring having an oxygen atom, a nitrogen atom, a sulfur atom, or the like. Examples of a skeleton of the aryl group having a heterocyclic ring include pyrrole, furan, thiophene, indole, benzofuran, and benzothiophene.

The alkyl group and cycloalkyl group of R²⁰⁴ and R²⁰⁵ are preferably a linear alkyl group having 1 to 10 carbon atoms or a branched alkyl group having 3 to 10 carbon atoms (for example, a methyl group, an ethyl group, a propyl group, a butyl group, and a pentyl group), or a cycloalkyl group having 3 to 10 carbon atoms (for example, a cyclopentyl group, a cyclohexyl group, and a norbornyl group).

The aryl group, the alkyl group, and the cycloalkyl group of R²⁰⁴ and R²⁰⁵ may each independently have a substituent. Examples of the substituent which may be included in each of the aryl group, the alkyl group, and the cycloalkyl group of R²⁰⁴ and R²⁰⁵ include an alkyl group (for example, having 1 to 15 carbon atoms), a cycloalkyl group (for example, having 3 to 15 carbon atoms), an aryl group (for example, having 6 to 15 carbon atoms), an alkoxy group (for example, having 1 to 15 carbon atoms), a halogen atom, a hydroxyl group, and a phenylthio group. In addition, it is also preferable that the substituents of R²⁰⁴ and R²⁰⁵ each independently form an acid-decomposable group by any combination of the substituents.

Specific examples of the organic cation are shown below, but the present invention is not limited thereto.

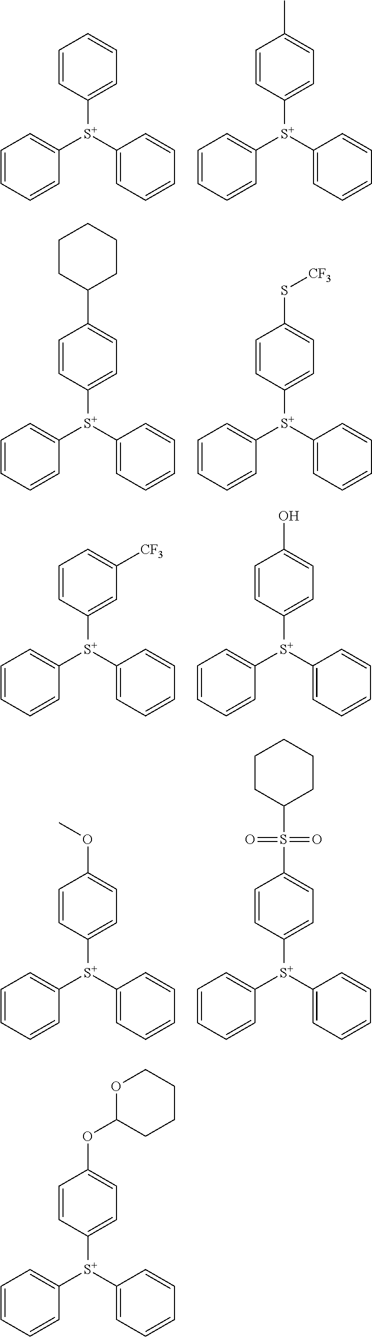

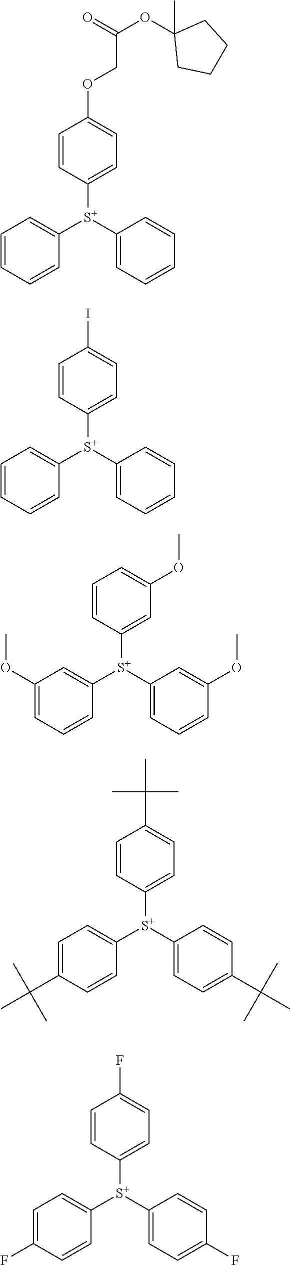

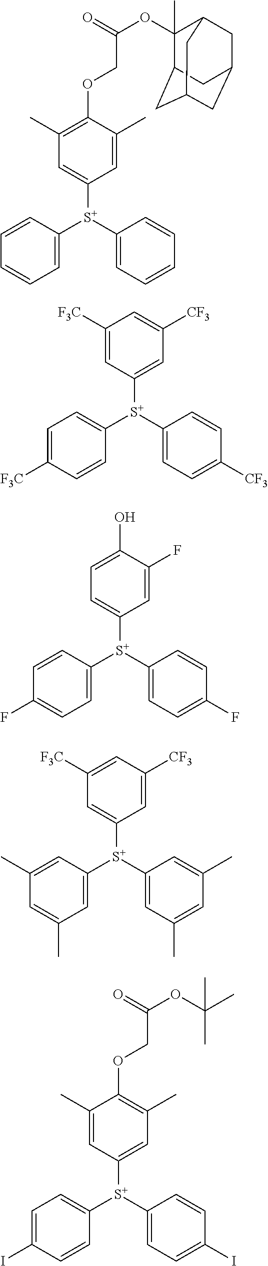

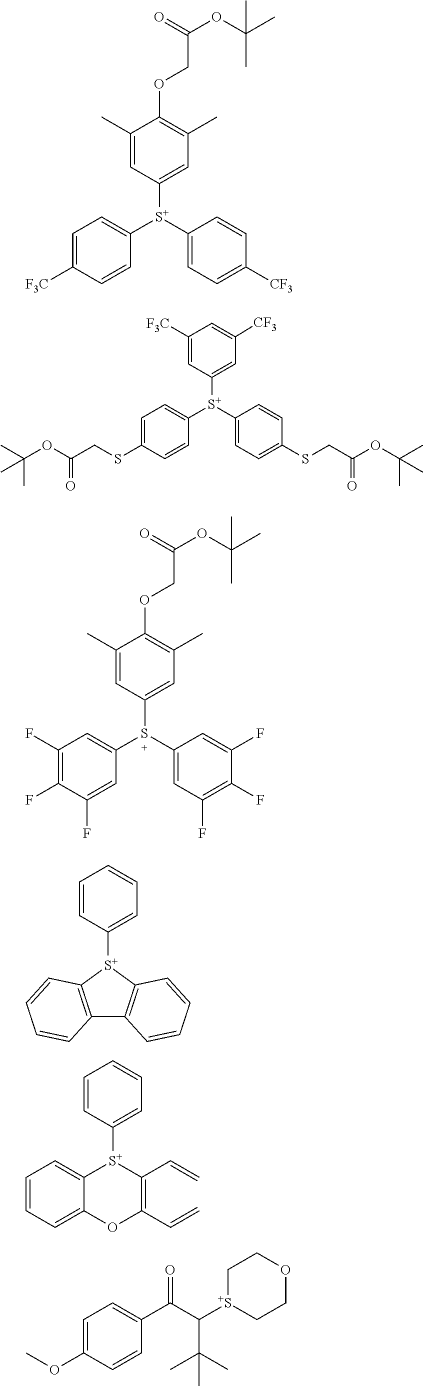

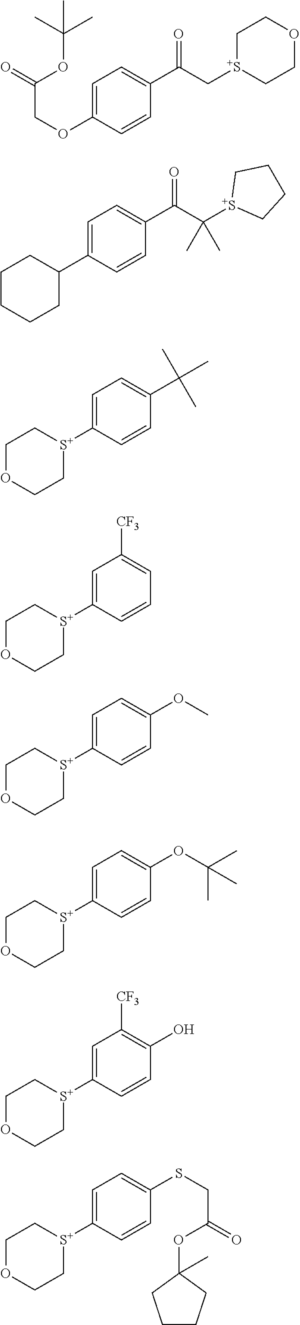

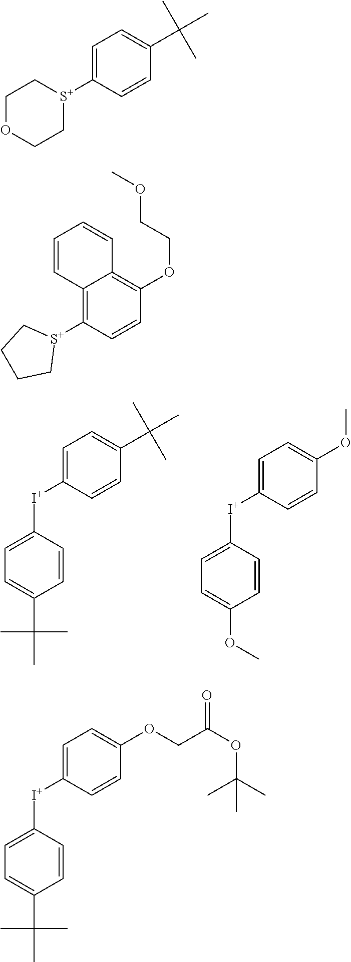 [SMILES: CC(C)(C)c1ccc([S+]2CCOCC2)cc1 ; COCCOc1ccc([S+]2CCCC2)c2ccccc12 ; CC(C)(C)c1ccc([I+]c2ccc(C(C)(C)C)cc2)cc1 ; COc1ccc([I+]c2ccc(OC)cc2)cc1 ; CC(C)(C)OC(=O)COc1ccc([I+]c2ccc(C(C)(C)C)cc2)cc1]

In the compound represented by “M⁺X⁻”, X⁻ represents an organic anion.

The organic anion is not particularly limited, and examples thereof include a monovalent or di- or higher valent organic anion.

The organic anion is preferably an anion with significantly lower ability to undergo nucleophilic reaction, and more preferably a non-nucleophilic anion.

The organic anion has the above-described partial structure represented by any of General Formula (1), (2), or (3).

Examples of the non-nucleophilic anion include a sulfonate anion (an aliphatic sulfonate anion, an aromatic sulfonate anion, a camphor sulfonate anion, and the like), a carboxylate anion (an aliphatic carboxylate anion, an aromatic carboxylate anion, an aralkyl carboxylate anion, and the like), a sulfonylimide anion, a bis(alkylsulfonyl)imide anion, and a tris(alkylsulfonyl)methide anion.

An aliphatic moiety in the aliphatic sulfonate anion and the aliphatic carboxylate anion may be a linear or branched alkyl group or a cycloalkyl group, and a linear or branched alkyl group having 1 to 30 carbon atoms or a cycloalkyl group having 3 to 30 carbon atoms is preferable.

The above-described alkyl group may be, for example, a fluoroalkyl group (which may have a substituent other than a fluorine atom and may be a perfluoroalkyl group).

An aryl group in the aromatic sulfonate anion and the aromatic carboxylate anion is preferably an aryl group having 6 to 14 carbon atoms, and examples thereof include a phenyl group, a tolyl group, and a naphthyl group.

The alkyl group, cycloalkyl group, and aryl group mentioned above may have a substituent. The substituent is not particularly limited, and examples thereof include a nitro group, a halogen atom such as a fluorine atom and a chlorine atom, a carboxyl group, a hydroxyl group, an amino group, a cyano group, an alkoxy group (preferably having 1 to 15 carbon atoms), an alkyl group (preferably having 1 to 10 carbon atoms), a cycloalkyl group (preferably having 3 to 15 carbon atoms), an aryl group (preferably having 6 to 14 carbon atoms), an alkoxycarbonyl group (preferably having 2 to 7 carbon atoms), an acyl group (preferably having 2 to 12 carbon atoms), an alkoxycarbonyloxy group (preferably having 2 to 7 carbon atoms), an alkylthio group (preferably having 1 to 15 carbon atoms), an alkylsulfonyl group (preferably having 1 to 15 carbon atoms), an alkyliminosulfonyl group (preferably having 1 to 15 carbon atoms), and an aryloxysulfonyl group (preferably having 6 to 20 carbon atoms).

As the aralkyl group in the aralkyl carboxylate anion, an aralkyl group having 7 to 14 carbon atoms is preferable.

Examples of the aralkyl group having 7 to 14 carbon atoms include a benzyl group, a phenethyl group, a naphthylmethyl group, a naphthylethyl group, and a naphthylbutyl group.

Examples of the sulfonylimide anion include a saccharin anion.

As the alkyl group in the bis(alkylsulfonyl)imide anion and the tris(alkylsulfonyl)methide anion, an alkyl group having 1 to 5 carbon atoms is preferable. Examples of a substituent of these alkyl group include a halogen atom, an alkyl group substituted with a halogen atom, an alkoxy group, an alkylthio group, an alkyloxysulfonyl group, an aryloxysulfonyl group, and a cycloalkylaryloxysulfonyl group, and a fluorine atom or an alkyl group substituted with a fluorine atom is preferable.

In addition, the alkyl groups in the bis(alkylsulfonyl)imide anion may be bonded to each other to form a ring structure. As a result, acid strength is increased.

Examples of other non-nucleophilic anions include fluorinated phosphorus (for example, PF₆⁻), fluorinated boron (for example, BF₄⁻), and fluorinated antimony (for example, SbF₆⁻).

As the non-nucleophilic anion, an aliphatic sulfonate anion in which at least an α-position of the sulfonic acid is substituted with a fluorine atom, an aromatic sulfonate anion substituted with a fluorine atom or a group having a fluorine atom, a bis(alkylsulfonyl)imide anion in which an alkyl group is substituted with a fluorine atom, or a tris(alkylsulfonyl)methide anion in which an alkyl group is substituted with a fluorine atom is preferable. Among these, a perfluoroaliphatic sulfonate anion (preferably having 4 to 8 carbon atoms) or benzenesulfonate anion having a fluorine atom is more preferable, and a nonafluorobutanesulfonate anion, a perfluorooctanesulfonate anion, a pentafluorobenzenesulfonate anion, or a 3,5-bis(trifluoromethyl)benzenesulfonate anion is still more preferable.

As the non-nucleophilic anion, an anion represented by Formula (AN1) is also preferable.

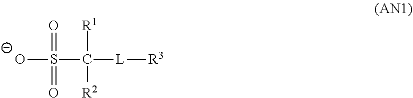

In Formula (AN1), R¹ and R² each independently represent a hydrogen atom or a substituent.

The substituent is not particularly limited, but a group which is not an electron withdrawing group is preferable. Examples of the group which is not an electron withdrawing group include a hydrocarbon group, a hydroxyl group, an oxyhydrocarbon group, an oxycarbonyl hydrocarbon group, an amino group, a hydrocarbon-substituted amino group, and a hydrocarbon-substituted amide group.

In addition, the groups which are not an electron withdrawing group are each independently preferably —R′, —OH, —OR′, —OCOR′, —NH₂, —NR′₂, —NHR′, or —NHCOR′. R′ is a monovalent hydrocarbon group.

Examples of the above-described monovalent hydrocarbon group represented by R′ include an alkyl group such as a methyl group, an ethyl group, a propyl group, and a butyl group; an alkenyl group such as an ethenyl group, a propenyl group, and a butenyl group; a monovalent linear or branched hydrocarbon group of an alkynyl group or the like, such as an ethynyl group, a propynyl group, and a butynyl group; a cycloalkyl group such as a cyclopropyl group, a cyclobutyl group, a cyclopentyl group, a cyclohexyl group, a norbornyl group, and an adamantyl group; a monovalent alicyclic hydrocarbon group of a cycloalkenyl group or the like, such as a cyclopropenyl group, a cyclobutenyl group, a cyclopentenyl group, and a norbornenyl group; an aryl group such as a phenyl group, a tolyl group, a xylyl group, a mesityl group, a naphthyl group, a methylnaphthyl group, an anthryl group, and a methylanthryl group; and a monovalent aromatic hydrocarbon group of an aralkyl group or the like, such as a benzyl group, a phenethyl group, a phenylpropyl group, a naphthylmethyl group, and an anthrylmethyl group.

Among these, R¹ and R² are each independently a hydrocarbon group (preferably, a cycloalkyl group) or a hydrogen atom.

L represents a divalent linking group.
In a case of a plurality of L's, L's may be the same or different from each other.
Examples of the divalent linking group include —O—CO—O—, —COO—, —CONH—, —CO—, —O—, —S—, —SO—, —SO₂—, an alkylene group (preferably having 1 to 6 carbon atoms), a cycloalkylene group (preferably having 3 to 15 carbon atoms), an alkenylene group (preferably having 2 to 6 carbon atoms), and a divalent linking group formed by a combination of a plurality of these groups. Among these, as the divalent linking group, —O—CO—O—, —COO—, —CONH—, —CO—, —O—, —SO₂—, —O—CO—O-alkylene group-, —COO-alkylene group-, or —CONH-alkylene group- is preferable, and —O—CO—O—, —O—CO—O-alkylene group-, —COO—, —CONH—, —SO₂—, —SO₂—O—, or —COO-alkylene group- is more preferable.

For example, L is preferably a group represented by Formula (AN1-1).

*^{a}—(CR^{2a}₂)_{X}-Q-(CR^{2b}₂)_{Y}—*^{b} (AN1-1)

In Formula (AN1-1), *^{a} represents a bonding position with R³ in Formula (AN1).

*^{b} represents a bonding position —C(R¹)(R²)— in Formula (AN1).

X and Y each independently represent an integer of 0 to 10, preferably an integer of 0 to 3.

R^{2a} and R^{2b} each independently represent a hydrogen atom or a substituent.

In a case where R^{2a}'s and R^{2b}'s are present in a plural number, the R^{2a}'s and R^{2b}'s which are present in a plural number may be the same or different from each other.

However, in a case where Y is 1 or more, R^{2b} in CR^{2b}₂ which is directly bonded to —C(R¹)(R²)— in Formula (AN1) is not a fluorine atom.

Q represents *^{A}—O—CO—O—*^{B}, *^{A}—CO—*^{B}, *^{A}—CO—O—*^{B}, *^{A}—O—CO—*^{B}, *^{A}—O—*^{B}, *^{A}—S—*^{B} or *^{A}—SO₂*^{B}.

However, in a case where X+Y in Formula (AN1-1) is 1 or more and R^{2a} and R^{2b} in Formula (AN1-1) are all hydrogen atoms, Q represents *^{A}—O—CO—O—*^{B}, *^{A}—CO—*^{B}, *^{A}—O—CO—*^{B}, *^{A}—O—*^{B}, *^{A}—S—*^{B}, or *^{A}—SO₂—*^{B}.

*^{A} represents a bonding position on the R³ side in Formula (AN1), and *^{B} represents a bonding position on the —SO₃⁻ side in Formula (AN1).

In Formula (AN1), R³ represents an organic group.

The above-described organic group is not particularly limited as long as it has one or more carbon atoms, and may be a linear group (for example, a linear alkyl group) or a branched group (for example, a branched alkyl group such as a t-butyl group), and may be a cyclic group. The above-described organic group may or may not have a substituent. The above-described organic group may or may not have a heteroatom (oxygen atom, sulfur atom, nitrogen atom, and/or the like).

Among these, R³ is preferably an organic group having a cyclic structure. The above-described cyclic structure may be monocyclic or polycyclic, and may have a substituent. The ring of the organic group including a cyclic structure is preferably directly bonded to L in Formula (AN1).

For example, the above-described organic group having a cyclic structure may or may not have a heteroatom (oxygen atom, sulfur atom, nitrogen atom, and/or the like). The heteroatom may be substituted on one or more carbon atoms forming the cyclic structure.

As the above-described organic group having a cyclic structure, for example, a hydrocarbon group having a cyclic structure, a lactone ring group, or a sultone ring group is preferable. Among these, the above-described organic group having a cyclic structure is preferably a hydrocarbon group having a cyclic structure.

The above-described hydrocarbon group having a cyclic structure is preferably a monocyclic or polycyclic cycloalkyl group. These groups may have a substituent.

The above-described cycloalkyl group may be a monocycle (cyclohexyl group or the like) or a polycycle (adamantyl group or the like), and the number of carbon atoms is preferably 5 to 12.

As the above-described lactone group and sultone group, for example, a group obtained by removing one hydrogen atom from ring member atoms constituting the lactone structure or the sultone structure in any of the structures represented by Formulae (LC1-1) to (LC1-21) described above and the structures represented by Formulae (SL1-1) to (SL1-3) described above is preferable.

Formula (AN1) has the above-described partial structure represented by any of General Formula (1), (2), or (3).

The non-nucleophilic anion may be a benzenesulfonate anion, and is preferably a benzenesulfonate anion substituted with a branched alkyl group or a cycloalkyl group.

As the non-nucleophilic anion, an anion represented by Formula (AN2) is also preferable.

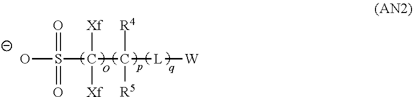

In Formula (AN2), o represents an integer of 1 to 3. p represents an integer of 0 to 10. q represents an integer of 0 to 10.

Xf represents a hydrogen atom, a fluorine atom, an alkyl group substituted with at least one fluorine atom, or an organic group not having a fluorine atom. The number of carbon atoms in the alkyl group is preferably 1 to 10 and more preferably 1 to 4. In addition, the alkyl group substituted with at least one fluorine atom is preferably a perfluoroalkyl group.

Xf is preferably a fluorine atom or a perfluoroalkyl group having 1 to 4 carbon atoms and more preferably a fluorine atom or CF₃, and it is still more preferable that both Xf's are fluorine atoms.

R⁴ and R⁵ each independently represent a hydrogen atom, a fluorine atom, an alkyl group, or an alkyl group substituted with at least one fluorine atom. In a case of a plurality of R⁴'s and R⁵'s, R⁴'s and R⁵'s may be the same or different from each other.

The alkyl group represented by R⁴ and R⁵ preferably has 1 to 4 carbon atoms. The above-described alkyl group may further have a substituent. R₄ and R₅ are preferably a hydrogen atom.

L represents a divalent linking group. The definition of L is synonymous with L in Formula (AN1).

W represents an organic group including a cyclic structure. Among these, a cyclic organic group is preferable.

Examples of the cyclic organic group include an alicyclic group, an aryl group, and a heterocyclic group.

The alicyclic group may be monocyclic or polycyclic. Examples of the monocyclic alicyclic group include a monocyclic cycloalkyl group such as a cyclopentyl group, a cyclohexyl group, and a cyclooctyl group. Examples of the polycyclic alicyclic group include a polycyclic cycloalkyl group such as a norbornyl group, a tricyclodecanyl group, a tetracyclodecanyl group, a tetracyclododecanyl group, and an adamantyl group. Among these, an alicyclic group having a bulky structure with 7 or more carbon atoms, such as a norbornyl group, a tricyclodecanyl group, a tetracyclodecanyl group, a tetracyclododecanyl group, and an adamantyl group, is preferable.

The aryl group may be monocyclic or polycyclic. Examples of the above-described aryl group include a phenyl group, a naphthyl group, a phenanthryl group, and an anthryl group.

The heterocyclic group may be monocyclic or polycyclic. Among these, in a case of a polycyclic heterocyclic group, the diffusion of the acid can be further suppressed. In addition, the heterocyclic group may or may not have aromaticity. Examples of a heterocycle having aromaticity include a furan ring, a thiophene ring, a benzofuran ring, a benzothiophene ring, a dibenzofuran ring, a dibenzothiophene ring, and a pyridine ring. Examples of a heterocycle not having aromaticity include a tetrahydropyran ring, a lactone ring, a sultone ring, and a decahydroisoquinoline ring. As the heterocycle in the heterocyclic group, a furan ring, a thiophene ring, a pyridine ring, or a decahydroisoquinoline ring is preferable.

The above-described cyclic organic group may have a substituent. Examples of the above-described substituent include an alkyl group (may be linear or branched; preferably having 1 to 12 carbon atoms), a cycloalkyl group (may be monocyclic, polycyclic, or spirocyclic; preferably having 3 to 20 carbon atoms), an aryl group (preferably having 6 to 14 carbon atoms), a hydroxyl group, an alkoxy group, an ester group, an amide group, a urethane group, a ureido group, a thioether group, a sulfonamide group, and a sulfonic acid ester group. A carbon constituting the cyclic organic group (carbon contributing to ring formation) may be a carbonyl carbon.

As the anion represented by Formula (AN2), SO₃⁻—CF₂—CH₂—OCO-(L)_{q′}-W, SO₃⁻—CF₂—CHF—CH₂—OCO-(L)_{q′}-W, SO₃⁻—CF₂—COO-(L)_{q′}-W, SO₃⁻—CF₂—CF₂—CH₂—CH₂-(L)_{q}-W, or SO₃⁻—CF₂—CH(CF₃)—OCO-(L)_{q′}-W is preferable. Here, L, q, and W are the same as in Formula (AN2). q′ represents an integer of 0 to 10.

Formula (AN2) has the above-described partial structure represented by any of General Formula (1), (2), or (3).

As the non-nucleophilic anion, an aromatic sulfonate anion represented by Formula (AN3) is also preferable.

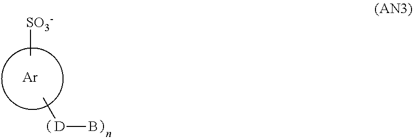

In Formula (AN3), Ar represents an aryl group (phenyl group or the like), and may further have a substituent other than a sulfonate anion and a -(D-B) group. Examples of the substituent which may be further included include a fluorine atom and a hydroxyl group.

n represents an integer of 0 or more. n is preferably 1 to 4, more preferably 2 or 3, and still more preferably 3.

D represents a single bond or a divalent linking group. Examples of the divalent linking group include an ether group, a thioether group, a carbonyl group, a sulfoxide group, a sulfone group, a sulfonic acid ester group, an ester group, and a group consisting of a combination of two or more of these groups.

B represents a hydrocarbon group.

B is preferably an aliphatic hydrocarbon group, and more preferably an isopropyl group, a cyclohexyl group, or an aryl group which may further have a substituent (such as a tricyclohexylphenyl group). B may have a substituent.

Formula (AN3) has the above-described partial structure represented by any of General Formula (1), (2), or (3).

As the non-nucleophilic anion, a methide anion represented by Formula (AN4) is also preferable.

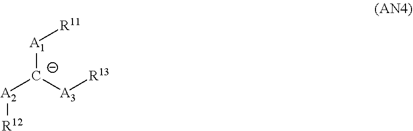

In Formula (AN4), R¹¹, R¹², and R¹³ each independently represent an organic group. A₁ to A₃ each independently represent —C(═O)— or —S(═O)₂—. At least two of R¹¹, R¹², and R¹³ may be bonded to each other to form a ring.

In Formula (AN4), R¹¹, R¹², and R¹³ each independently represent an organic group.

The above-described organic group is not particularly limited as long as it has one or more carbon atoms, and may be a linear group (for example, a linear alkyl group) or a branched group (for example, a branched alkyl group such as a t-butyl group), and may be a cyclic group. The above-described organic group may or may not have a substituent. The above-described organic group may or may not have a heteroatom (oxygen atom, sulfur atom, nitrogen atom, and/or the like).

Among these, the above-described organic group is preferably an organic group having a cyclic structure. The above-described cyclic structure may be monocyclic or polycyclic, and may have a substituent.

For example, the above-described organic group having a cyclic structure may or may not have a heteroatom (oxygen atom, sulfur atom, nitrogen atom, and/or the like). The heteroatom may be substituted on one or more carbon atoms forming the cyclic structure.

As the above-described organic group having a cyclic structure, for example, a hydrocarbon group having a cyclic structure, a lactone ring group, or a sultone ring group is preferable. Among these, the above-described organic group having a cyclic structure is preferably a hydrocarbon group having a cyclic structure.

The above-described hydrocarbon group having a cyclic structure is preferably a monocyclic or polycyclic cycloalkyl group, or an aryl group. These groups may have a substituent.

The above-described cycloalkyl group may be a monocycle (cyclohexyl group or the like) or a polycycle (adamantyl group or the like), and the number of carbon atoms is preferably 5 to 12.

The aryl group may be monocyclic or polycyclic. Examples of the above-described aryl group include a phenyl group, a naphthyl group, a phenanthryl group, and an anthryl group.

As the above-described lactone group and sultone group, for example, a group obtained by removing one hydrogen atom from ring member atoms constituting the lactone structure or the sultone structure in any of the structures represented by Formulae (LC1-1) to (LC1-21) described above and the structures represented by Formulae (SL1-1) to (SL1-3) described above is preferable.

The above-described organic group having a cyclic structure may have a substituent.

Formula (AN4) has the above-described partial structure represented by any of General Formula (1), (2), or (3).

As the non-nucleophilic anion, a disulfonamide anion is also preferable.

The disulfonamide anion is, for example, an anion represented by N⁻(SO₂—R^{q})₂.

Here, R^{q} represents an alkyl group which may have a substituent, and is preferably a fluoroalkyl group and more preferably a perfluoroalkyl group. Two R^{q}'s may be bonded to each other to form a ring. The group formed by bonding two R^{q}'s to each other is preferably an alkylene group which may have a substituent, more preferably a fluoroalkylene group, and still more preferably a perfluoroalkylene group. The number of carbon atoms in the above-described alkylene group is preferably 2 to 4.

R^{q} has the above-described partial structure represented by any of General Formula (1), (2), or (3).

In addition, examples of the non-nucleophilic anion include anions represented by Formulae (d1-1) to (d1-4).

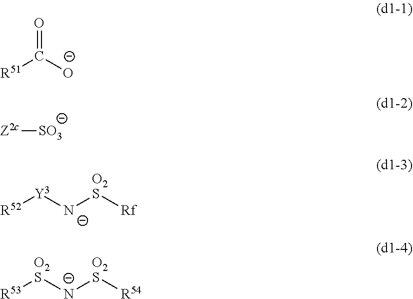

In Formula (d1-1), R⁵¹ represents a hydrocarbon group (for example, an aryl group such as a phenyl group) which may have a substituent (for example, a hydroxyl group).

In Formula (d1-2), Z^{2c} represents a hydrocarbon group having 1 to 30 carbon atoms, which may have a substituent (provided that a carbon atom adjacent to S is not substituted with a fluorine atom).

The above-described divalent hydrocarbon group in Z^{2c} may be linear or branched, may have a cyclic structure. In addition, carbon atoms in the above-described hydrocarbon group (preferably, carbon atoms which are ring member atoms in a case where the above-described hydrocarbon group has a cyclic structure) may be a carbonyl carbon (—CO—). Examples of the hydrocarbon group include a group having a norbornyl group which may have a substituent. Carbon atoms forming the above-described norbornyl group may be a carbonyl carbon.

In addition, it is preferable that “Z^{2c}—S₃⁻” in Formula (d1-2) is different from the above-described anions represented by Formulae (AN1) to (AN3). For example, Z^{2c} is preferably a group other than an aryl group. In addition, for example, in Z^{2c}, atoms at α-position and β-position with respect to —SO₃⁻ are preferably atoms other than carbon atoms having a fluorine atom as a substituent. For example, in Z^{2c}, it is preferable that the atom at α-position and/or the atom at β-position with respect to —SO₃⁻ is a ring member atom in a cyclic group.

In Formula (d1-3), R⁵² represents an organic group (preferably, a hydrocarbon group having a fluorine atom), Y³ represents a linear, branched, or cyclic alkylene group, an arylene group, or a carbonyl group, and Rf represents a hydrocarbon group.

In Formula (d1-4), R⁵³ and R⁵⁴ each independently represent an organic group (preferably, a hydrocarbon group having a fluorine atom). R⁵³ and R⁵⁴ may be bonded to each other to form a ring.

The anions represented by Formulae (d1-1) to (d1-4) have the above-described partial structure represented by any of General Formula (1), (2), or (3).

The organic anion may be used alone or in combination of two or more kinds thereof.

It is also preferable that the photoacid generator is at least one selected from the group consisting of compounds (I) and (II).

(Compound (I))

The compound (I) is a compound having one or more of the following structural moieties X and one or more of the following structural moieties Y, in which the compound generates an acid including the following first acidic moiety derived from the following structural moiety X and the following second acidic moiety derived from the following structural moiety Y by irradiation with actinic ray or radiation.

Structural moiety X: a structural moiety which consists of an anionic moiety A₁⁻ and a cationic moiety M₁⁺, and forms a first acidic moiety represented by HA₁ by irradiation with actinic ray or radiation

Structural moiety Y: a structural moiety which consists of an anionic moiety A₂⁻ and a cationic moiety M₂, and forms a second acidic moiety represented by HA₂ by irradiation with actinic ray or radiation

In addition, the above-described compound (I) satisfies the following condition I.

Condition I: a compound PI, which is formed by, in the compound (I), replacing the cationic moiety M₁⁺ in the structural moiety X and the cationic moiety M₂⁺ in the structural moiety Y with H⁺, has an acid dissociation constant a1 derived from the acidic moiety represented by HA₁, formed by replacing the cationic moiety M₁⁺ in the structural moiety X with H⁺, and has an acid dissociation constant a2 derived from the acidic moiety represented by HA₂, formed by replacing the cationic moiety M₂⁺ in the structural moiety Y with H⁺, in which the acid dissociation constant a2 is larger than the acid dissociation constant a1

Hereinafter, the condition I will be described in more detail.

In a case where the compound (I) is, for example, a compound that generates an acid having one first acidic moiety derived from the above-described structural moiety X and one second acidic moiety derived from the above-described structural moiety Y, the compound PI corresponds to “compound having HA₁ and HA₂”.

As the acid dissociation constant a1 and the acid dissociation constant a2 of such a compound PI, more specifically, in a case of obtaining acid dissociation constants of the compound PI, a pKa in a case where the compound PI is to be “compound having A₁⁻ and HA₂” is defined as the acid dissociation constant a1, and a pKa in a case where the “compound having A₁⁻ and HA₂” is to be “compound having A₁⁻ and A₂⁻” is defined as the acid dissociation constant a2.

In addition, in a case where the compound (I) is, for example, a compound that generates an acid having two first acidic moieties derived from the above-described structural moiety X and one second acidic moiety derived from the above-described structural moiety Y, the compound PI corresponds to “compound having two HA_{I}'s and one HA₂”.

In a case of obtaining acid dissociation constants of such a compound PI, an acid dissociation constant in a case where the compound PI is to be “compound having one A₁⁻, one HA₁, and one HA₂” and an acid dissociation constant in a case where the “compound having one A₁⁻, one HA₁, and one HA₂” is to be “compound having two A₁⁻ and one HA₂” correspond to the above-described acid dissociation constant a1. In addition, an acid dissociation constant in a case where the “compound having two A₁⁻ and one HA₂” is to be “compound having two A₁⁻ and one A₂⁻” corresponds to the acid dissociation constant a2. That is, in the compound PI, in a case of a plurality of acid dissociation constants derived from the acidic moiety represented by HA₁, which is formed by replacing the above-described cationic moiety M₁⁺ in the above-described structural moiety X with H⁺, a value of the acid dissociation constant a2 is larger than the largest value of the plurality of acid dissociation constants a1. In a case where the acid dissociation constant in a case where the compound PI is to be the “compound having one A₁⁻, one HA₁, and one HA₂” is defined as aa, and the acid dissociation constant in a case where the “compound having one A₁⁻, one HA₁, and one HA₂” is to be the “compound having two A₁⁻ and one HA₂” is defined as ab, a relationship between aa and ab satisfies aa<ab.

The acid dissociation constant a1 and the acid dissociation constant a2 can be obtained by the above-described method for measuring an acid dissociation constant.

The above-described compound PI corresponds to an acid generated in a case where the compound (I) is irradiated with actinic ray or radiation.

In a case where the compound (I) has two or more of the structural moieties X, the structural moieties X may be the same or different from each other. In addition, two or more of A₁⁻'s and two or more of M₁⁺ may be the same or different from each other.

In addition, in the compound (I), A₁⁻ and A₂⁻, and M₁⁺ and M₂⁺ may be the same or different from each other, but it is preferable that A₁⁻ and A₂⁻ are different from each other.

In the above-described compound PI, a difference (absolute value) between the acid dissociation constant a1 (in a case of a preferably of acid dissociation constants a1, the maximum value thereof) and the acid dissociation constant a2 is preferably 0.1 or more, more preferably 0.5 or more, and still more preferably 1.0 or more. The upper limit value of the difference (absolute value) between the acid dissociation constant a1 (in a case of a preferably of acid dissociation constants a1, the maximum value thereof) and the acid dissociation constant a2 is not particularly limited, but is, for example, 16 or less.

In the above-described compound PI, the acid dissociation constant a2 is, for example, 20 or less, preferably 15 or less. The lower limit value of the acid dissociation constant a2 is preferably −4.0 or more.

In addition, in the above-described compound PI, the acid dissociation constant a1 is, for example, 2.0 or less, preferably 0 or less. The lower limit value of the acid dissociation constant a1 is preferably −20.0 or more.

The anionic moiety A₁ and the anionic moiety A₂⁻ are structural moieties including a negatively charged atom or atomic group, and examples thereof include structural moieties selected from the group consisting of Formulae (AA-1) to (AA-3), and Formulae (BB-1) to (B1B-6).

The anionic moiety A₁⁻ is preferably an acidic moiety capable of forming an acidic moiety having a small acid dissociation constant, and among these, any one of Formulae (AA-1) to (AA-3) is more preferable and any one of Formula (AA-1) or (AA-3) is still more preferable.

In addition, the anionic moiety A2- is preferably an acidic moiety capable of forming an acidic moiety having a larger acid dissociation constant than the anionic moiety A₁⁻, and any one of Formulae (BB-1) to (BB-6) is more preferable and any one of Formula (BB-1) or (BB-4) is still more preferable.

In Formulae (AA-1) to (AA-3) and Formulae (BB-1) to (BB-6), * represents a bonding position.

In Formula (AA-2), R^{A} represents a monovalent organic group. The monovalent organic group represented by R^{A} is not particularly limited, and examples thereof include a cyano group, a trifluoromethyl group, and a methanesulfonyl group.

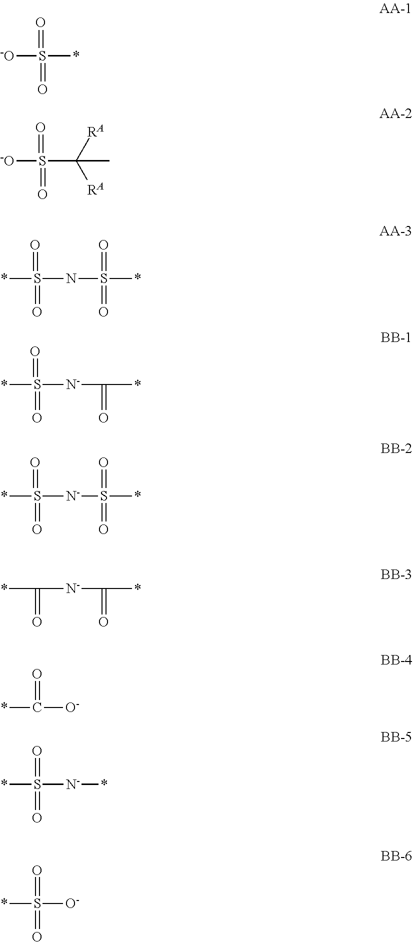

In addition, the cationic moiety M₁⁺ and the cationic moiety M₂⁺ are a structural moiety including a positively charged atom or atomic group, and examples thereof include an organic cation having a charge of 1. Examples of the organic cation include the organic cation represented by M⁺ described above.

The compound (I) has the above-described partial structure represented by any of General Formula (1), (2), or (3) in the anionic moiety.

A specific structure of the compound (I) is not particularly limited, and examples thereof include compounds represented by Formulae (Ia-1) to (Ia-5) described below.

—Compound Represented by Formula (Ia-1)—

Hereinafter, first, the compound represented by Formula (Ia-1) will be described.

M₁₁⁺A₁₁⁻-L₁-A₁₂⁻M₁₂⁺ (Ia-1)

The compound represented by Formula (Ia-1) generates an acid represented by HA₁₁-L₁-A₁₂H by irradiation with actinic ray or radiation.

In Formula (Ia-1), M₁₁⁺ and M₁₂⁺ each independently represent an organic cation.

A₁₁⁻ and A₁₂⁻ each independently represent a monovalent anionic functional group.

L₁ represents a divalent linking group.

M₁₁⁺ and M₁₂⁺ may be the same or different from each other.

A₁₁⁻ and A₁₂⁻ may be the same or different from each other, but it is preferable to be different from each other.

However, in the compound PIa (HA₁₁-L₁-A₁₂H) formed by replacing cations represented by M₁₁⁺ and M₁₂⁺ with H⁺ in Formula (Ia-1), the acid dissociation constant a2 derived from the acidic moiety represented by A₁₂H is larger than the acid dissociation constant a1 derived from the acidic moiety represented by HA₁₁. Suitable values of the acid dissociation constant a1 and the acid dissociation constant a2 are as described above. In addition, the acid generated from the compound PIa and the acid generated from the compound represented by Formula (Ia-1) by irradiation with actinic ray or radiation are the same.

In addition, at least one of M₁₁⁺, M₁₂⁺, A₁₁⁻, A₁₂⁻, or L₁ may have an acid-decomposable group as a substituent.

In Formula (Ia-1), examples of each organic cation represented by M₁₁⁺ and M₁₂⁺ include the above-described organic cation represented by M⁺ described above.

The monovalent anionic functional group represented by A₁₁⁻ is intended to be a monovalent group including the above-described anionic moiety A₁⁻. In addition, the monovalent anionic functional group represented by A₁₂⁻ is intended to be a monovalent group including the above-described anionic moiety A₂⁻.

As the monovalent anionic functional group represented by A₁₁⁻ and A₁₂⁻, a monovalent anionic functional group including the anionic moiety of any of Formulae (AA-1) to (AA-3) and Formulae (BB-1) to (BB-6) described above is preferable, and a monovalent anionic functional group selected from the group consisting of Formulae (AX-1) to (AX-3) and Formulae (BX-1) to (BX-7) is more preferable.

Among these, as the monovalent anionic functional group represented by A₁₁⁻, a monovalent anionic functional group represented by any of Formulae (AX-1) to (AX-3) is preferable.

In addition, as the monovalent anionic functional group represented by A₁₂⁻, a monovalent anionic functional group represented by any of Formulae (BX-1) to (BX-7) is preferable, and a monovalent anionic functional group represented by any of Formulae (BX-1) to (BX-6) is more preferable.

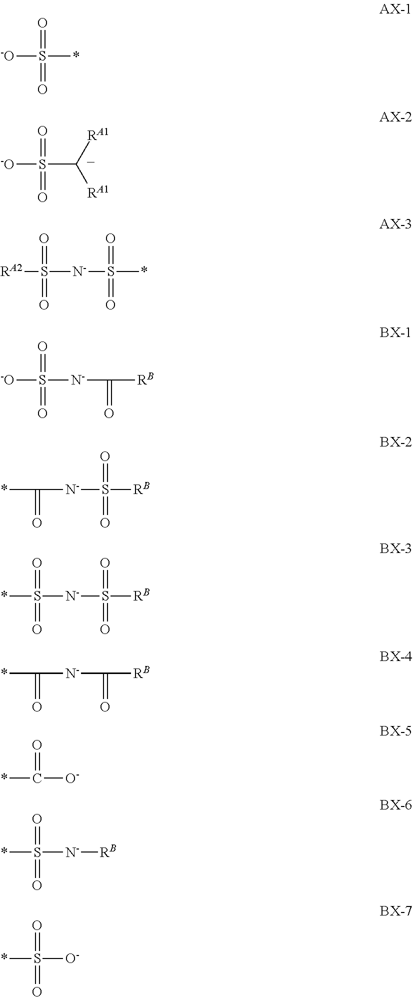

In Formulae (AX-1) to (AX-3), R^{A1} and R^{A2} each independently represent a monovalent organic group. * represents a bonding position.

The monovalent organic group represented by R^{A1} is not particularly limited, and examples thereof include a cyano group, a trifluoromethyl group, and a methanesulfonyl group.

As the monovalent organic group represented by R^{A2}, a linear or branched alkyl group, a cycloalkyl group, or an aryl group is preferable.

The number of carbon atoms in the above-described alkyl group is preferably 1 to 15, more preferably 1 to 10, and still more preferably 1 to 6.

The above-described alkyl group may have a substituent. As the substituent, a fluorine atom or a cyano group is preferable, and a fluorine atom is more preferable. In a case where the above-described alkyl group has a fluorine atom as the substituent, the substituent may be a perfluoroalkyl group.

The above-described cycloalkyl group may be a monocycle (cyclohexyl group or the like) or a polycycle (adamantyl group or the like), and the number of carbon atoms is preferably 3 to 15, more preferably 3 to 10, and still more preferably 3 to 6.

The above-described cycloalkyl group may have a substituent. As the substituent, a fluorine atom or a cyano group is preferable, and a fluorine atom is more preferable.

The above-described aryl group is preferably a phenyl group or a naphthyl group, and more preferably a phenyl group.

The above-described aryl group may have a substituent. As the substituent, a fluorine atom, an iodine atom, a perfluoroalkyl group (for example, preferably having 1 to 10 carbon atoms and more preferably having 1 to 6 carbon atoms), or a cyano group is preferable, and a fluorine atom, an iodine atom, or a perfluoroalkyl group is more preferable.

In Formulae (BX-1) to (BX-4) and (BX-6), R^{B} represents a monovalent organic group. * represents a bonding position.

As the monovalent organic group represented by R^{B}, a linear or branched alkyl group, a cycloalkyl group, or an aryl group is preferable.

The number of carbon atoms in the above-described alkyl group is preferably 1 to 15, more preferably 1 to 10, and still more preferably 1 to 6.

The above-described alkyl group may have a substituent. The substituent is not particularly limited, but a fluorine atom or a cyano group is preferable, and a fluorine atom is more preferable. In a case where the above-described alkyl group has a fluorine atom as the substituent, the substituent may be a perfluoroalkyl group.

In a case where the carbon atom to be the bonding position in the alkyl group (for example, in the case of Formulae (BX-1) and (BX-4), a carbon atom directly bonded to —CO— specified in the alkyl group of the formulae; in the case of Formulae (BX-2) and (BX-3), a carbon atom directly bonded to —SO₂— specified in the alkyl group of the formulae; and in the case of Formula (BX-6), a carbon atom directly bonded to N-specified in the alkyl group of the formula) has a substituent, the substituent is also preferably a fluorine atom or a substituent other than a cyano group.

In addition, the above-described alkyl group may have a carbon atom substituted with a carbonyl carbon.

The above-described cycloalkyl group may be a monocycle (cyclohexyl group or the like) or a polycycle (adamantyl group or the like), and the number of carbon atoms is preferably 3 to 15, more preferably 3 to 10, and still more preferably 3 to 6.

The above-described cycloalkyl group may have a substituent. As the substituent, a fluorine atom or a cyano group is preferable, and a fluorine atom is more preferable.

In a case where the carbon atom to be the bonding position in the cycloalkyl group (for example, in the case of Formulae (BX-1) and (BX-4), a carbon atom directly bonded to —CO— specified in the cycloalkyl group of the formulae; in the case of Formulae (BX-2) and (BX-3), a carbon atom directly bonded to —SO₂— specified in the cycloalkyl group of the formulae; and in the case of Formula (BX-6), a carbon atom directly bonded to N- specified in the cycloalkyl group of the formula) has a substituent, the substituent is also preferably a fluorine atom or a substituent other than a cyano group.

In addition, the above-described cycloalkyl group may have a carbon atom substituted with a carbonyl carbon.

The above-described aryl group is preferably a phenyl group or a naphthyl group, and more preferably a phenyl group.

The above-described aryl group may have a substituent. As the substituent, a fluorine atom, an iodine atom, a perfluoroalkyl group (for example, preferably having 1 to 10 carbon atoms and more preferably having 1 to 6 carbon atoms), a cyano group, an alkyl group (for example, preferably having 1 to 10 carbon atoms and more preferably having 1 to 6 carbon atoms), an alkoxy group (for example, preferably having 1 to 10 carbon atoms and more preferably having 1 to 6 carbon atoms), or an alkoxycarbonyl group (for example, preferably having 2 to 10 carbon atoms and more preferably having 2 to 6 carbon atoms) is preferable; and a fluorine atom, an iodine atom, a perfluoroalkyl group, an alkyl group, an alkoxy group, or an alkoxycarbonyl group is more preferable.

In Formula (Ia-1), the divalent liking group represented by L₁ is not particularly limited, and examples thereof include —CO—, —NR—, —CO—, —O—, —S—, —SO—, —SO₂—, an alkylene group (preferably having 1 to 6 carbon atoms; may be linear or branched), a cycloalkylene group (preferably having 3 to 15 carbon atoms), an alkenylene group (preferably having 2 to 6 carbon atoms), a divalent aliphatic heterocyclic group (preferably a 5- to 10-membered ring, more preferably a 5- to 7-membered ring, and still more preferably a 5- or 6-membered ring; each having at least one of an N atom, an O atom, an S atom, or an Se atom in the ring structure), and a divalent aromatic heterocyclic group (preferably a 5- to 10-membered ring, more preferably a 5- to 7-membered ring, and still more preferably a 5- or 6-membered ring; each having at least one of an N atom, an O atom, an S atom, or an Se atom in the ring structure), a divalent aromatic hydrocarbon ring group (preferably a 6- to 10-membered ring, and more preferably a 6-membered ring), and a divalent linking group formed by a combination of a plurality of these groups. Examples of R include a hydrogen atom and a monovalent organic group. The monovalent organic group is not particularly limited, but is preferably, for example, an alkyl group (preferably having 1 to 6 carbon atoms).

The above-described alkylene group, the above-described cycloalkylene group, the above-described alkenylene group, and the above-described divalent aliphatic heterocyclic group, divalent aromatic heterocyclic group, and divalent aromatic hydrocarbon ring group may have a substituent. Examples of the substituent include a halogen atom (preferably, a fluorine atom).

Among these, as the divalent linking group represented by L₁, a divalent linking group represented by Formula (L1) is preferable.

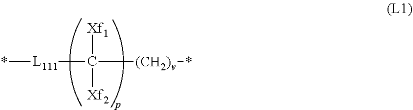

In Formula (L1), L₁₁₁ represents a single bond or a divalent linking group.

The divalent linking group represented by L₁₁₁ is not particularly limited, and examples thereof include —CO—, —NH—, —O—, —SO—, —SO₂—, an alkylene group which may have a substituent (preferably having 1 to 6 carbon atoms; may be linear or branched), a cycloalkylene group which may have a substituent (preferably having 3 to 15 carbon atoms), an aryl group which may have a substituent (preferably having 6 to 10 carbon atoms), and a divalent linking group formed by a combination of a plurality of these groups. The substituent is not particularly limited, and examples thereof include a halogen atom.

p represents an integer of 0 to 3, and preferably represents an integer of 1 to 3.

v represents an integer of 0 or 1.

Xf₁'s each independently represent a fluorine atom or an alkyl group substituted with at least one fluorine atom. The number of carbon atoms in the alkyl group is preferably 1 to 10 and more preferably 1 to 4. In addition, the alkyl group substituted with at least one fluorine atom is preferably a perfluoroalkyl group.

Xf₂'s each independently represent a hydrogen atom, an alkyl group which may have a fluorine atom as a substituent, or a fluorine atom. The number of carbon atoms in the alkyl group is preferably 1 to 10 and more preferably 1 to 4. Among these, Xf₂ preferably represents a fluorine atom or an alkyl group substituted with at least one fluorine atom, and more preferably represents a fluorine atom or a perfluoroalkyl group.

Among these, as each of Xf₁ and Xf₂, a fluorine atom or a perfluoroalkyl group having 1 to 4 carbon atoms is preferable, and a fluorine atom or CF₃ is more preferable. In particular, it is still more preferable that both Xf₁ and Xf₂ are fluorine atoms.

* represents a bonding position.

In a case where L₁₁ in Formula (Ia-1) represents the divalent linking group represented by Formula (L1), it is preferable that the bonding site (*) on the L₁₁₁ side of Formula (L1) is bonded to A₁₂⁻ of Formula (Ia-1).

The anionic moiety in Formula (Ia-1) has the above-described partial structure represented by any of General Formula (1), (2), or (3).

—Compounds Represented by Formulae (Ia-2) to (Ia-4)—

Next, the compounds represented by Formulae (Ia-2) to (Ia-4) will be described.

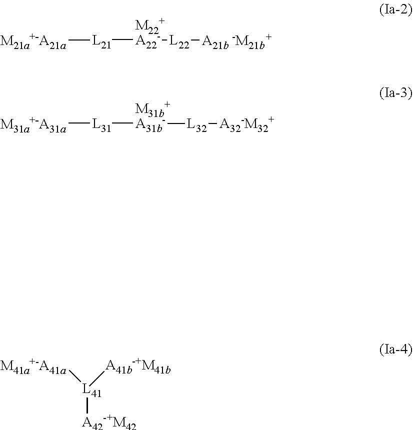

In Formula (Ia-2), A₂₁a and A21b- each independently represent a monovalent anionic functional group. Here, the monovalent anionic functional group represented by A₂₁ₐ⁻ and A_{21b}⁻ is intended to be a monovalent group including the above-described anionic moiety A₁⁻. The monovalent anionic functional group represented by A₂₁ₐ⁻ and A_{21b}⁻ is not particularly limited, and examples thereof include the monovalent anionic functional group selected from the group consisting of Formulae (AX-1) to (AX-3) described above.

A₂₂⁻ represents a divalent anionic functional group. Here, the divalent anionic functional group represented by A₂₂⁻ is intended to be a divalent group including the above-described anionic moiety A₂⁻. Examples of the divalent anionic functional group represented by A₂₂⁻ include a divalent anionic functional group represented by Formulae (BX-8) to (BX-11).

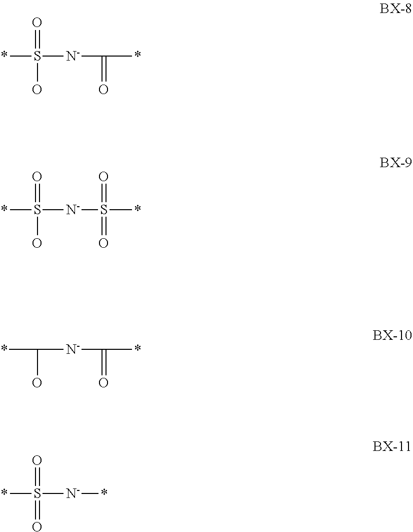

M₂₁ₐ⁺, M_{21b}⁺, and M₂₂⁺ each independently represent an organic cation. The organic cation represented by M₂₁ₐ⁺, M_{21b}⁺, and M₂₂⁺ has the same meaning as M₁₁⁺ described above, and a suitable aspect thereof is also the same.

L₂₁ and L₂₂ each independently represent a divalent organic group.

In addition, in a compound PIa-2 of Formula (Ia-2), in which the organic cation represented by M₂₁ₐ⁺, M_{21b}⁺, and M₂₂⁺ is replaced with H⁺, an acid dissociation constant a2 derived from an acidic moiety represented by A₂₂H is larger than an acid dissociation constant a1-1 derived from A₂₁ₐH and an acid dissociation constant a1-2 derived from an acidic moiety represented by A_{21b}H. The acid dissociation constant a1-1 and the acid dissociation constant a1-2 correspond to the above-described acid dissociation constant a1.

A₂₁ₐ⁻ and A_{21b}⁻ may be the same or different from each other. In addition, M₂₁ₐ⁺, M_{21b}⁺, and M₂₂⁺ may be the same or different from each other.

In addition, at least one of M₂₁ₐ⁺, M_{21b}⁺, M₂₂⁺, A₂₁ₐ⁻, A_{21b}⁻, L₂₁, or L₂₂ may have an acid-decomposable group as a substituent.

In Formula (Ia-3), A₃₁ₐ⁻ and A₃₂⁻ each independently represent a monovalent anionic functional group. The definition of the monovalent anionic functional group represented by A₃₁ₐ⁻ is the same as A₂₁ₐ⁻ and A_{21b}⁻ in Formula (Ia-2), and a suitable aspect thereof is also the same.

The monovalent anionic functional group represented by A₃₂⁻ is intended to be a monovalent group including the above-described anionic moiety A₂⁻. The monovalent anionic functional group represented by A₃₂⁻ is not particularly limited, and examples thereof include the monovalent anionic functional group selected from the group consisting of Formulae (BX-1) to (BX-7) described above.

A_{31b}⁻ represents a divalent anionic functional group. Here, the divalent anionic functional group represented by A_{31b}⁻ is intended to be a divalent group including the above-described anionic moiety A₁⁻. Examples of the divalent anionic functional group represented by A_{31b}⁻ include a divalent anionic functional group represented by Formula (AX-4).

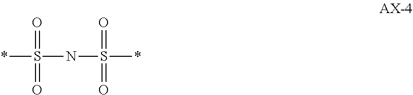 [SMILES: *S(=O)(=O)NS(*)(=O)=O]

M₃₁ₐ⁺, M_{31b}⁺, and M₃₂⁺ each independently represent a monovalent organic cation. The organic cation represented by M₃₁ₐ⁺, M_{31b}⁺, and M₃₂⁺ has the same meaning as M₁₁⁺ described above, and a suitable aspect thereof is also the same.

L₃₁ and L₃₂ each independently represent a divalent organic group.

In addition, in a compound PIa-3 of Formula (Ia-3), in which the organic cation represented by M₃₁ₐ⁺, M_{31b}⁺, and M₃₂⁺ is replaced with H⁺, an acid dissociation constant a2 derived from an acidic moiety represented by A₃₂H is larger than an acid dissociation constant a1-3 derived from an acidic moiety represented by A₃₁ₐH and an acid dissociation constant a1-4 derived from an acidic moiety represented by A_{31b}H. The acid dissociation constant a1-3 and the acid dissociation constant a1-4 correspond to the above-described acid dissociation constant a1.

A₃₁ₐ⁺ and A₃₂⁻ may be the same or different from each other. In addition, M₃₁ₐ⁺, M_{31b}⁺, and M₃₂⁺ may be the same or different from each other.

In addition, at least one of M₃₁ₐ⁺, M_{31b}⁺, M₃₂⁺, A₃₁ₐ⁻, A₃₂⁻, L₃₁, or L₃₂ may have an acid-decomposable group as a substituent.

In Formula (Ia-4), A₄₁ₐ⁻, A_{41b}⁻, and A₄₂⁻ each independently represent a monovalent anionic functional group. The definition of the monovalent anionic functional group represented by A₄₁ₐ⁻ and A_{41b}⁻ is the same as A₂₁ₐ⁻ and A_{21b}⁻ in Formula (Ia-2). In addition, the definition of the monovalent anionic functional group represented by A₄₂⁻ is the same as A₃₂⁻ in Formula (Ia-3), and a suitable aspect thereof is also the same.

M₄₁ₐ⁺, M_{41b}⁺, and M₄₂⁺ each independently represent an organic cation. The organic cation represented by M₄₁ₐ⁺, M_{41b}⁺, and M₄₂⁺ has the same meaning as M₁₁⁺ described above, and a suitable aspect thereof is also the same.

L₄₁ represents a trivalent organic group.

In addition, in a compound PIa-4 of Formula (Ia-4), in which the organic cation represented by M₄₁ₐ⁺, M_{41b}⁺, and M₄₂⁺ is replaced with H⁺, an acid dissociation constant a2 derived from an acidic moiety represented by A₄₂H is larger than an acid dissociation constant a1-5 derived from an acidic moiety represented by A₄₁ₐH and an acid dissociation constant a1-6 derived from an acidic moiety represented by A_{41b}H. The acid dissociation constant a1-5 and the acid dissociation constant a1-6 correspond to the above-described acid dissociation constant a1.

A₄₁ₐ⁻, A_{41b}⁻, and A₄₂⁻ may be the same or different from each other. In addition, M₄₁ₐ⁺, M_{41b}⁺, and M₄₂⁺ may be the same or different from each other.

In addition, at least one of M₄₁ₐ⁺, M_{41b}⁺, M₄₂⁺, A₄₁ₐ⁻, A_{41b}⁻, A₄₂⁻, or L₄₁ may have an acid-decomposable group as a substituent.

The divalent organic group represented by L₂₁ and L₂₂ in Formula (Ia-2) and L₃₁ and L₃₂ in Formula (Ia-3) is not particularly limited, and examples thereof include —CO—, —NR—, —O—, —S—, —SO—, —SO₂—, an alkylene group (preferably having 1 to 6 carbon atoms; may be linear or branched), a cycloalkylene group (preferably having 3 to 15 carbon atoms), an alkenylene group (preferably having 2 to 6 carbon atoms), a divalent aliphatic heterocyclic group (preferably a 5- to 10-membered ring, more preferably a 5- to 7-membered ring, and still more preferably a 5- or 6-membered ring; each having at least one of an N atom, an O atom, an S atom, or an Se atom in the ring structure), and a divalent aromatic heterocyclic group (preferably a 5- to 10-membered ring, more preferably a 5- to 7-membered ring, and still more preferably a 5- or 6-membered ring; each having at least one of an N atom, an O atom, an S atom, or an Se atom in the ring structure), a divalent aromatic hydrocarbon ring group (preferably a 6- to 10-membered ring, and more preferably a 6-membered ring), and a divalent organic group formed by a combination of a plurality of these groups. Examples of R include a hydrogen atom and a monovalent organic group. The monovalent organic group is not particularly limited, but is preferably, for example, an alkyl group (preferably having 1 to 6 carbon atoms).

The above-described alkylene group, the above-described cycloalkylene group, the above-described alkenylene group, and the above-described divalent aliphatic heterocyclic group, divalent aromatic heterocyclic group, and divalent aromatic hydrocarbon ring group may have a substituent. Examples of the substituent include a halogen atom (preferably, a fluorine atom).

As the divalent organic group represented by L₂₁ and L₂₂ in Formula (Ia-2) and L₃₁ and L₃₂ in Formula (Ia-3), for example, a divalent organic group represented by Formula (L2) is also preferable.

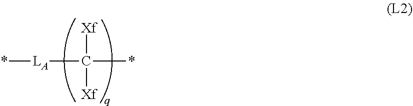

In Formula (L2), q represents an integer of 1 to 3. * represents a bonding position.

Xf's each independently represent a fluorine atom or an alkyl group substituted with at least one fluorine atom. The number of carbon atoms in the alkyl group is preferably 1 to 10 and more preferably 1 to 4. In addition, the alkyl group substituted with at least one fluorine atom is preferably a perfluoroalkyl group.

Xf is preferably a fluorine atom or a perfluoroalkyl group having 1 to 4 carbon atoms, and more preferably a fluorine atom or CF₃. In particular, it is still more preferable that both Xf's are fluorine atoms.

L_{A} represents a single bond or a divalent linking group.

The divalent linking group represented by L_{A} is not particularly limited, and examples thereof include —CO—, —O—, —SO—, —SO₂—, an alkylene group (preferably having 1 to 6 carbon atoms; may be linear or branched), a cycloalkylene group (preferably having 3 to 15 carbon atoms), a divalent aromatic hydrocarbon ring group (preferably a 6- to 10-membered ring, and more preferably a 6-membered ring), and a divalent linking group formed by a combination of a plurality of these groups.

In addition, the above-described alkylene group, the above-described cycloalkylene group, and the divalent aromatic hydrocarbon ring group may have a substituent. Examples of the substituent include a halogen atom (preferably, a fluorine atom).

Examples of the divalent organic group represented by Formula (L2) include *—CF₂—*, *—CF₂—CF₂—*, *—CF₂—CF₂—CF₂—*, *-Ph-O—SO₂—CF₂—*, *-Ph-O—SO₂—CF₂—CF₂—*, *-Ph-O—SO₂—CF₂—CF₂—CF₂—*, and *-Ph-OCO—CF₂—*. Ph is a phenylene group which may have a substituent, and is preferably a 1,4-phenylene group. The substituent is not particularly limited, and an alkyl group (for example, preferably having 1 to 10 carbon atoms and more preferably having 1 to 6 carbon atoms) or an alkoxy group (for example, preferably having 1 to 10 carbon atoms and more preferably having 1 to 6 carbon atoms), or an alkoxycarbonyl group (for example, preferably having 2 to 10 carbon atoms and more preferably having 2 to 6 carbon atoms) is preferable.

In a case where L₂₁ and L₂₂ in Formula (Ia-2) represents the divalent organic group represented by Formula (L2), it is preferable that the bonding site (*) on the L_{A} side of Formula (L2) is bonded to A₂₁ₐ⁻ and A_{21b}⁻ of Formula (Ia-2).

In addition, in a case where L₃₁ and L₃₂ in Formula (Ia-3) represents the divalent organic group represented by Formula (L2), it is preferable that the bonding site (*) on the L_{A} side of Formula (L2) is bonded to A₃₁ₐ⁻ and A₃₂⁻ of Formula (Ia-3).

The anionic moieties in Formulae (Ia-2) to (Ia-4) each independently have the above-described partial structure represented by any of General Formula (1), (2), or (3).

—Compound Represented by Formula (Ia-5)—

Next, Formula (Ia-5) will be described.

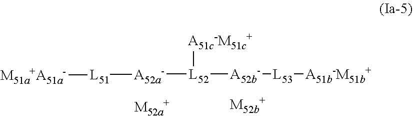

In Formula (Ia-5), A₅₁ₐ⁻, A_{51b}⁻, and A_{51c}⁻ each independently represent a monovalent anionic functional group. Here, the monovalent anionic functional group represented by A₅₁ₐ⁻, A_{51b}⁻, and A_{51c}⁻ is intended to be a monovalent group including the above-described anionic moiety A₁⁻. The monovalent anionic functional group represented by A₅₁ₐ⁻, A_{51b}⁻, and A_{51c}⁻ is not particularly limited, and examples thereof include the monovalent anionic functional group selected from the group consisting of Formulae (AX-1) to (AX-3) described above.

A₅₂ₐ⁻ and A_{52b}⁻ represents a divalent anionic functional group. Here, the divalent anionic functional group represented by A₅₂ₐ⁻ and A_{52b}⁻ is intended to be a divalent group including the above-described anionic moiety A₂⁻. Examples of the divalent anionic functional group represented by A₂₂⁻ include the divalent anionic functional group selected from the group consisting of Formulae (BX-8) to (BX-11) described above.

M₅₁ₐ⁺, M_{51b}⁻, M_{51c}⁺, M₅₂ₐ⁺, and M_{52b}⁺ each independently represent an organic cation. The organic cation represented by M₅₁ₐ⁺, M_{51b}⁺, M_{51c}⁺, M₅₂ₐ⁺, and M₅₂ₐ⁺ has the same meaning as M₁₁⁺ described above, and a suitable aspect thereof is also the same.

L₅₁ and L₅₃ each independently represent a divalent organic group. The divalent organic group represented by L₅₁ and L₅₃ has the same meaning as L₂₁ and L₂₂ in Formula (Ia-2) described above, and a suitable aspect thereof is also the same.

L₅₂ represents a trivalent organic group. The trivalent organic group represented by L₅₂ has the same meaning as L₄₁ in Formula (Ia-4) described above, and a suitable aspect thereof is also the same.

In addition, in a compound PIa-5 of Formula (Ia-5), in which the organic cation represented by M₅₁ₐ⁺, M_{51b}⁺, M_{51c}⁺, M₅₂ₐ⁺, and M_{52b}⁺ is replaced with H⁺, an acid dissociation constant a2-1 derived from an acidic moiety represented by A₅₂ₐH and an acid dissociation constant a2-2 derived from an acidic moiety represented by A_{52b}H are larger than an acid dissociation constant a1-1 derived from an acidic moiety represented by A₅₁ₐH, an acid dissociation constant a1-2 derived from an acidic moiety represented by A_{51b}H, and an acid dissociation constant a1-3 derived from an acidic moiety represented by A_{51c}H. The acid dissociation constants a1-1 to a1-3 correspond to the above-described acid dissociation constant a1, and the acid dissociation constants a2-1 and a2-2 correspond to the above-described acid dissociation constant a2.

A₅₁ₐ⁺, A_{51b}⁺, and A_{51c}⁺ may be the same or different from each other. In addition, A₅₂ₐ⁺ and A_{52b}⁻ may be the same or different from each other. In addition, M₅₁ₐ⁺, M_{51b}⁺, M_{51c}⁺, M₅₂ₐ⁺, and M_{52b}⁺ may be the same or different from each other.

In addition, at least one of M_{51b}⁺, M_{51c}⁺, M₅₂ₐ⁺, M_{52b}⁺, A₅₁ₐ⁻, A_{51b}⁻, A_{51c}⁻, L₅₁, L₅₂, or L₅₃ may have an acid-decomposable group as a substituent.

The anionic moiety in Formula (Ia-5) has the above-described partial structure represented by any of General Formula (1), (2), or (3).

(Compound (II))

The compound (II) is a compound having two or more of the structural moieties X and one or more of the following structural moieties Z, in which the compound generates an acid including two or more of the above-described first acidic moieties derived from the above-described structural moiety X and a structural moiety Z by irradiation with actinic ray or radiation.

Structural Moiety Z: A Non-Ionic Moiety Capable of Neutralizing an Acid

In the compound (II), the definition of the structural moiety X and the definitions of A₁⁻ and M₁⁺ are the same as the definition of the structural moiety X and the definitions of A₁⁻ and M₁⁺ in the above-described compound (I), and suitable aspects thereof are also the same.

In a compound PII formed by replacing the above-described cationic moiety M₁⁺ in the above-described structural moiety X with H⁺ in the above-described compound (II), a suitable range of an acid dissociation constant a1 derived from the acidic moiety represented by HA₁, formed by replacing the above-described cationic moiety M₁⁺ in the above-described structural moiety X with H⁺, is the same as in the acid dissociation constant a1 of the above-described compound PI.

In a case where the compound (II) is, for example, a compound which generates an acid having two of the above-described first acidic moieties derived from the above-described structural moiety X and the above-described structural moiety Z, the compound PII corresponds to “compound having two HA₁”. In a case of obtaining acid dissociation constants of the compound PII, an acid dissociation constant in a case where the compound PII is to be “compound having one A₁⁻ and one HA₁” and an acid dissociation constant in a case where the “compound having one A₁⁻ and one HA₁” is to be “compound having two A₁⁻” correspond to the acid dissociation constant a1.

The acid dissociation constant a1 can be obtained by the above-described method for measuring an acid dissociation constant.

The above-described compound PII corresponds to an acid generated in a case where the compound (II) is irradiated with actinic ray or radiation.

The above-described two or more of the structural moieties X may be the same or different from each other. In addition, two or more of A₁⁻'s and two or more of M₁⁺ may be the same or different from each other.

The non-ionic moiety capable of neutralizing an acid in the structural moiety Z is not particularly limited, and is preferably, for example, a moiety including a functional group having a group or an electron which is capable of electrostatically interacting with a proton.

Examples of the functional group having a group or electron capable of electrostatically interacting with a proton include a functional group with a macrocyclic structure, such as a cyclic polyether, or a functional group having a nitrogen atom having an unshared electron pair not contributing to π-conjugation. For example, the nitrogen atom having the unshared electron pair, which does not contribute to the π-conjugation, is a nitrogen atom having a partial structure represented by the following formula.

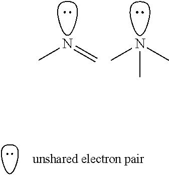

Examples of the partial structure of the functional group having a group or electron which is capable of electrostatically interacting with a proton include a crown ether structure, an azacrown ether structure, primary to tertiary amine structures, a pyridine structure, an imidazole structure, and a pyrazine structure, and among these, primary to tertiary amine structures are preferable.

The compound (II) has the above-described partial structure represented by any of General Formula (1), (2), or (3) in the anionic moiety.

The compound (II) is not particularly limited, and examples thereof include compounds represented by Formula (IIa-1) and Formula (IIa-2).

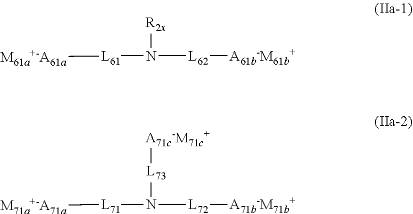

In Formula (IIa-1), A₆₁ₐ⁻ and A_{61b}⁻ have the same meaning as A₁₁⁻ in Formula (Ia-1) described above, and suitable aspects thereof are also the same. In addition, M₆₁ₐ⁺ and M_{61b}⁺ have the same meaning as M₁₁⁺ in Formula (Ia-1) described above, and suitable aspects thereof are also the same.

In Formula (IIa-1), L₆₁ and L₆₂ have the same meaning as L₁ in Formula (Ia-1) described above, and suitable aspects thereof are also the same.

In Formula (IIa-1), R_{2X} represents a monovalent organic group. The monovalent organic group represented by R_{2X} is not particularly limited, and examples thereof include an alkyl group (which preferably has 1 to 10 carbon atoms, and may be linear or branched), a cycloalkyl group (preferably having 3 to 15 carbon atoms), and an alkenyl group (preferably having 2 to 6 carbon atoms), in which —CH₂— may be substituted with one or a combination of two or more selected from the group consisting of —CO—, —NH—, —O—, —S—, —SO—, and —SO₂—.

In addition, the above-described alkylene group, the above-described cycloalkylene group, and the above-described alkenylene group may have a substituent. The substituent is not particularly limited, and examples thereof include a halogen atom (preferably, a fluorine atom).

In addition, in a compound PIIa-1 of Formula (IIa-1), in which the organic cation represented by M₆₁ₐ⁺ and M_{61b}⁺ is replaced with H⁺, an acid dissociation constant a1-7 derived from an acidic moiety represented by A₆₁ₐH and an acid dissociation constant a1-8 derived from an acidic moiety represented by A_{61b}H correspond to the above-described acid dissociation constant a1.

The compound PIIa-1 formed by replacing the above-described cationic moieties M₆₁ₐ⁺ and M_{61b}⁺ in the above-described structural moiety X with H⁺ in the Formula (IIa-1) corresponds to HA₆₁ₐ-L₆₁-N(R_{2X})-L₆₂-A_{61b}H. In addition, the acid generated from the compound PIIa-1 and the acid generated from the compound represented by Formula (IIa-1) by irradiation with actinic ray or radiation are the same.

In addition, at least one of M₆₁ₐ⁺, M_{61b}⁺, A₆₁ₐ⁻, A_{61b}⁻, L₆₁, L₆₂, or R_{2X} may have an acid-decomposable group as a substituent.

Formula (IIa-1) has the above-described partial structure represented by any of General Formula (1), (2), or (3) in the anionic moiety.

In Formula (IIa-2), A₇₁ₐ⁻, A_{71b}⁻, and A_{71c}⁻ have the same meaning as A₁₁⁻ in Formula (Ia-1) described above, and suitable aspects thereof are also the same. In addition, M₇₁ₐ⁺, M_{71b}⁺, and M_{71c}⁺ have the same meaning as M₁₁⁺ in Formula (Ia-1) described above, and suitable aspects thereof are also the same.

In Formula (IIa-2), L₇₁, L₇₂, and L₇₃ have the same meaning as L₁ in Formula (Ia-1) described above, and suitable aspects thereof are also the same.

In addition, in a compound PIIa-2 of Formula (IIa-2), in which the organic cation represented by M₇₁ₐ⁺, M_{71b}⁺, and M_{71c}⁺ is replaced with H⁺, an acid dissociation constant a1-9 derived from an acidic moiety represented by A₇₁ₐH, an acid dissociation constant a1-10 derived from an acidic moiety represented by A_{71b}H, and an acid dissociation constant a1-11 derived from an acidic moiety represented by A_{71c}H correspond to the above-described acid dissociation constant a1.

The compound PIIa-2 formed by replacing the above-described cationic moieties M₇₁ₐ⁺, M_{71b}⁺, and M_{71c}⁺ in the above-described structural moiety X with H⁺ in the Formula (IIa-2) corresponds to HA₇₁ₐ-L₇₁-N(L₇₃-A_{71c}H)-L₇₂-A_{71b}H. In addition, the acid generated from the compound PIIa-2 and the acid generated from the compound represented by Formula (IIa-2) by irradiation with actinic ray or radiation are the same.

In addition, at least one of M₇₁ₐ⁺, M_{71b}⁺, M_{71c}⁺, A₇₁ₐ⁺, A_{71b}⁻, A_{71c}⁻, L₇₁, L₇₂, or L₇₃ may have an acid-decomposable group as a substituent.

Formula (IIa-2) has the above-described partial structure represented by any of General Formula (1), (2), or (3) in the anionic moiety.

Examples of the moiety other than the cation, which can be included in the photoacid generator (B), are shown below.

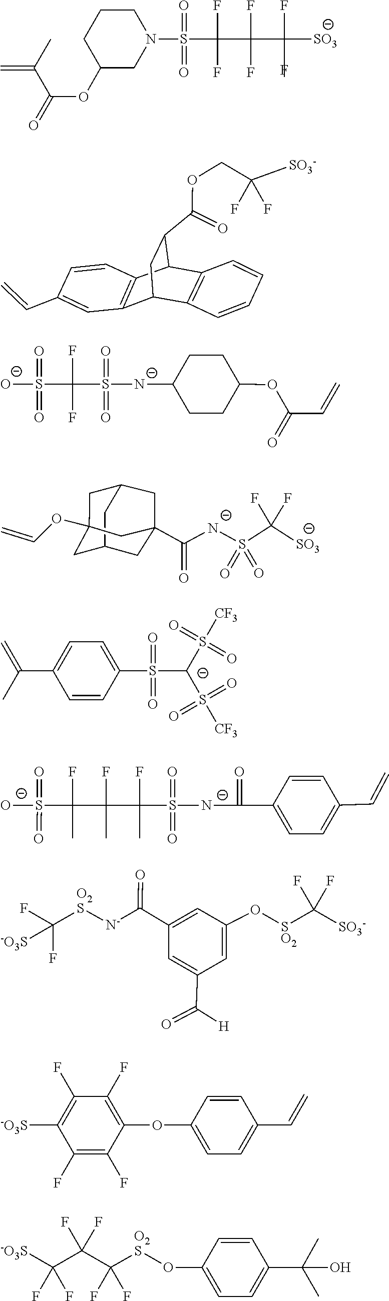

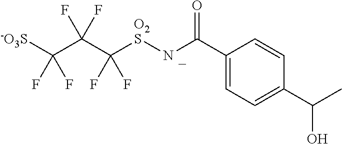

Specific examples of the photoacid generator are shown below, but the present invention is not limited thereto. In the following compounds, the anion and the cation can be optionally exchanged.

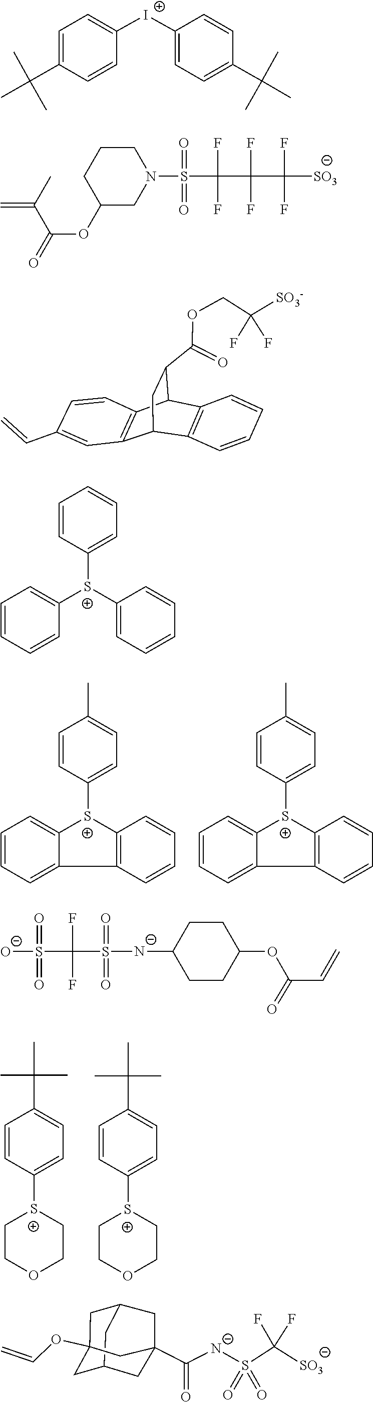

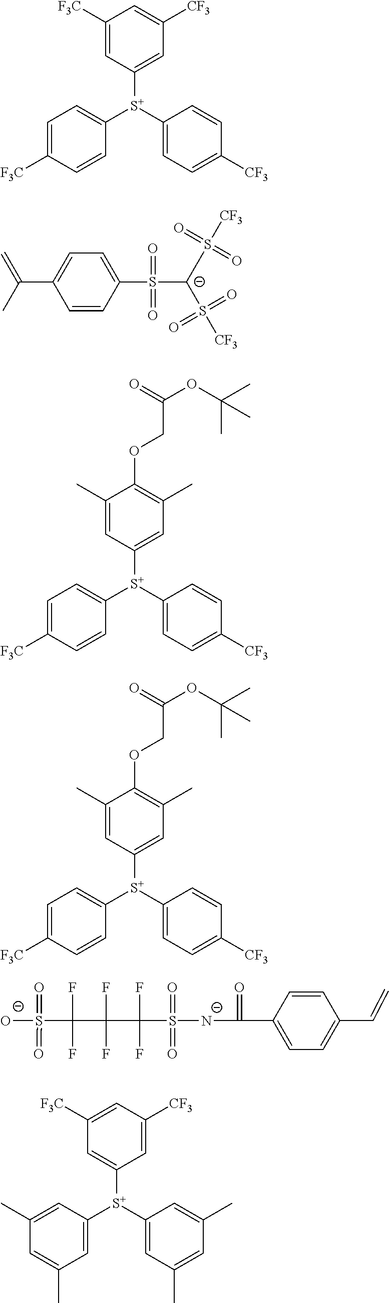

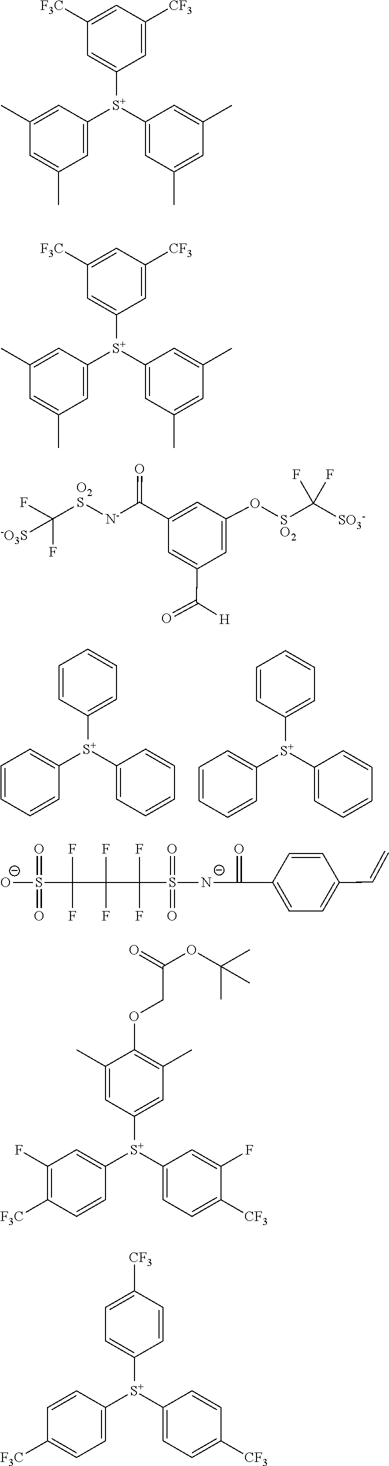

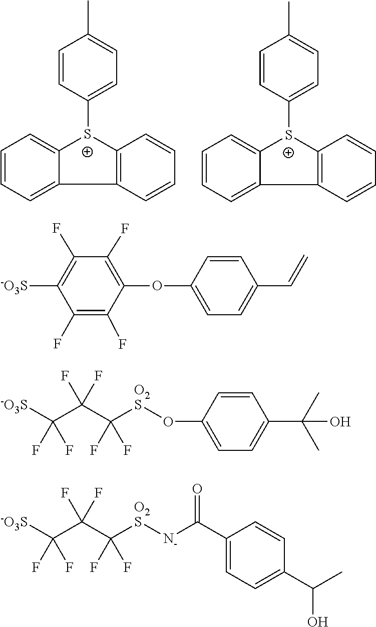

From the viewpoint that the effects of the present invention are more excellent, the photoacid generator (B) preferably has a sulfonamide structure.

As a preferred aspect, the photoacid generator (B) preferably has a sulfonamide structure in the anionic moiety.

Examples of the sulfonamide structure include the following structures.

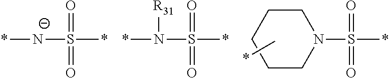

R₃₁ represents a hydrogen atom or an organic group.

* represents a bonding position.

The organic group is not particularly limited, and examples thereof include an organic group having 1 to 20 carbon atoms.

A content of the photoacid generator (B) in the composition according to the embodiment of the present invention is not particularly limited, but from the viewpoint that the effects of the present invention are more excellent, the content is preferably 1.0% by mass or more, more preferably 2.0% by mass or more, and still more preferably 5.0% by mass or more with respect to the total solid content of the composition. In addition, the above-described content is preferably 70.0% by mass or less, more preferably 60.0% by mass or less, and still more preferably 50.0% by mass or less.

The photoacid generator (B) may be used alone or in combination of two or more kinds thereof.

As long as effects of the present invention are not impaired, the composition according to the embodiment of the present invention may contain a compound (C) which is different from the photoacid generator and generates an acid by irradiation with actinic ray or radiation (also referred to as a compound (C) or a photoacid generator (C)), in addition to the photoacid generator (B).

The compound (C) is not particularly limited as long as it is a compound different from the photoacid generator (B), and examples thereof include a compound which does not have the above-described partial structure represented by any of General Formula (1), (2), or (3) in the photoacid generator (B).

In the above-described compounds (I) and (II), examples of the moiety other than the cation, which can be included in the compound not having the above-described partial structure represented by any of General Formula (1), (2), or (3), are shown below.

In addition, as the cation in the above-described compound, the organic cation mentioned as M⁺ in the above-described photoacid generator (B) can be used.

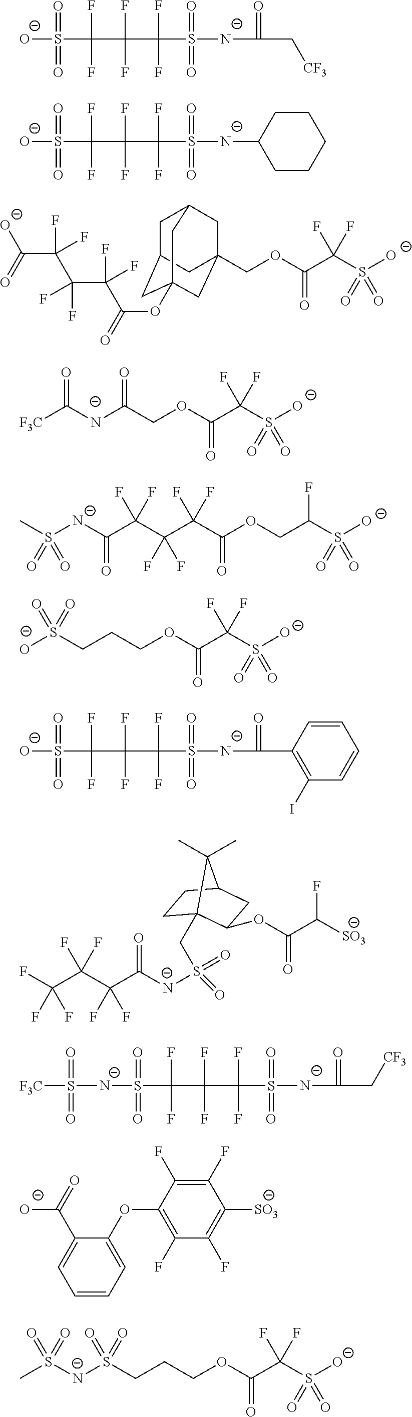

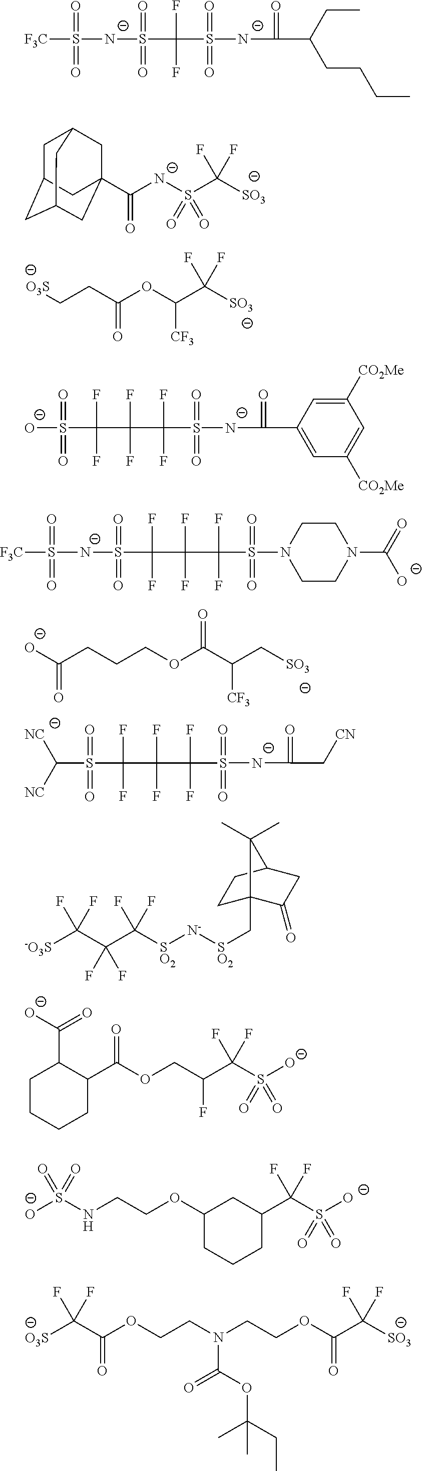

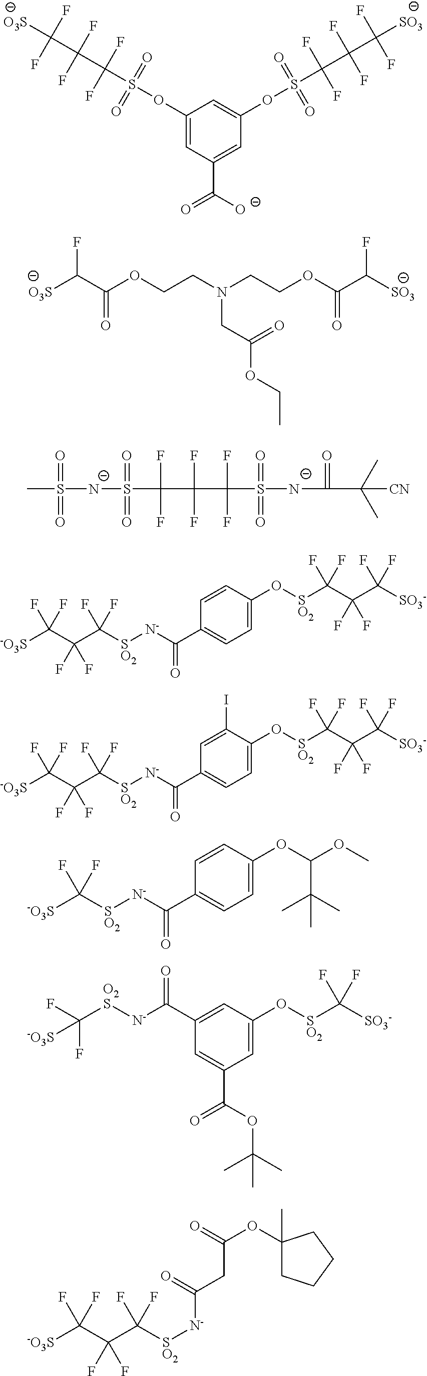

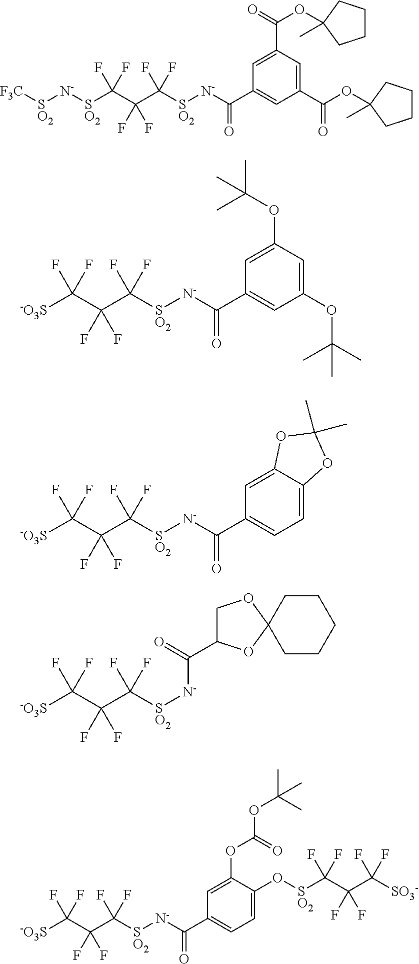

In addition, specific examples of the photoacid generator (C) include the following compounds. In the following compounds, the anion and the cation can be optionally exchanged.

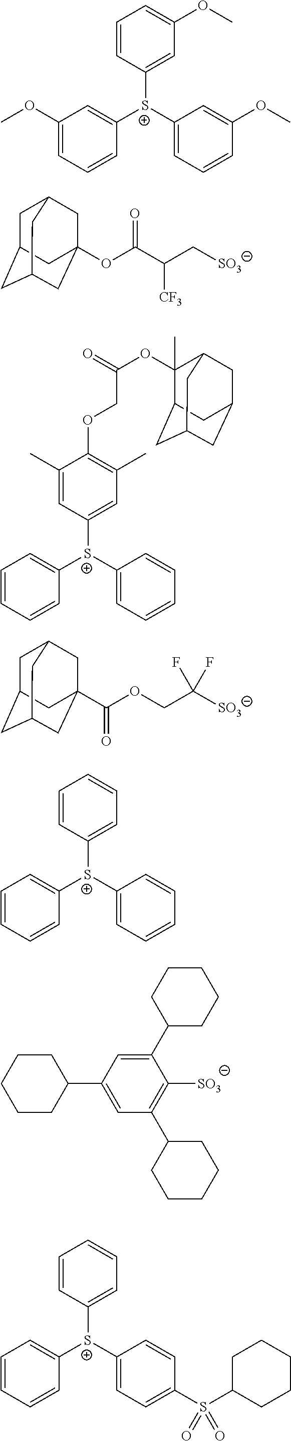

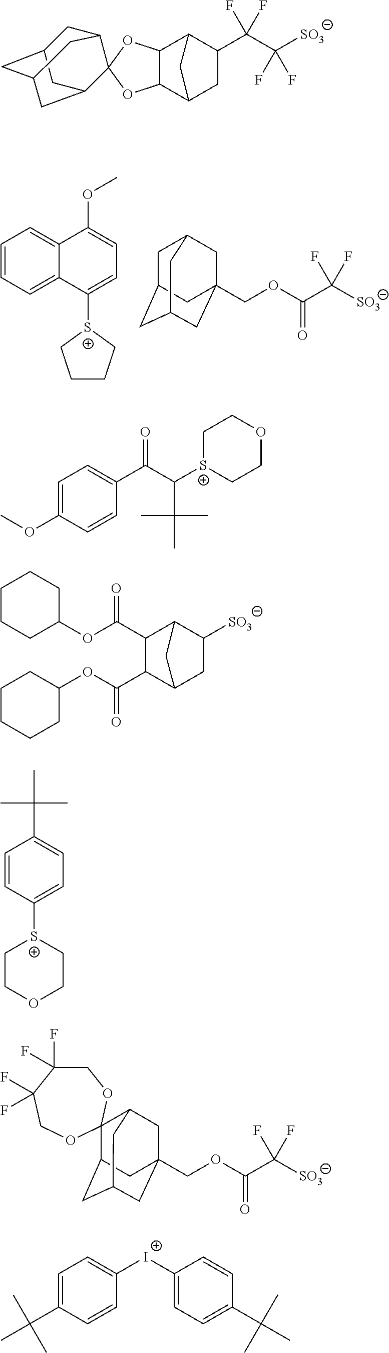

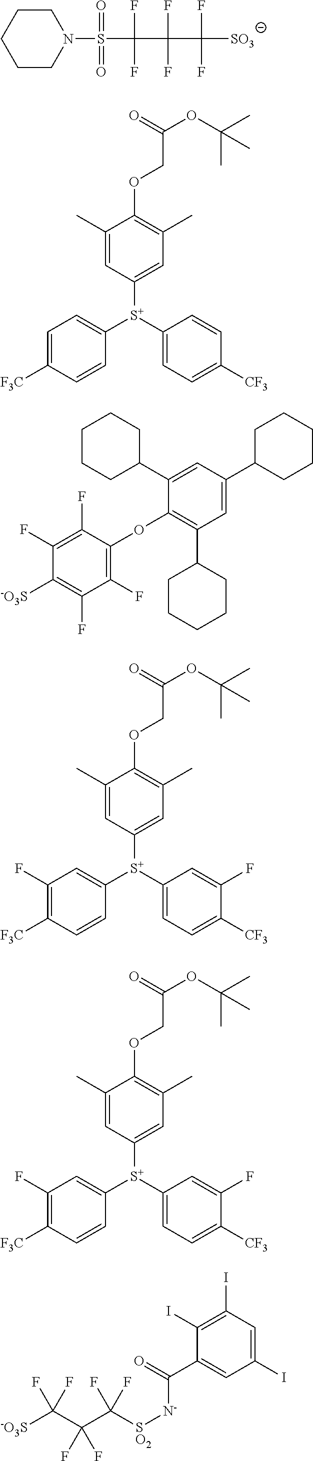

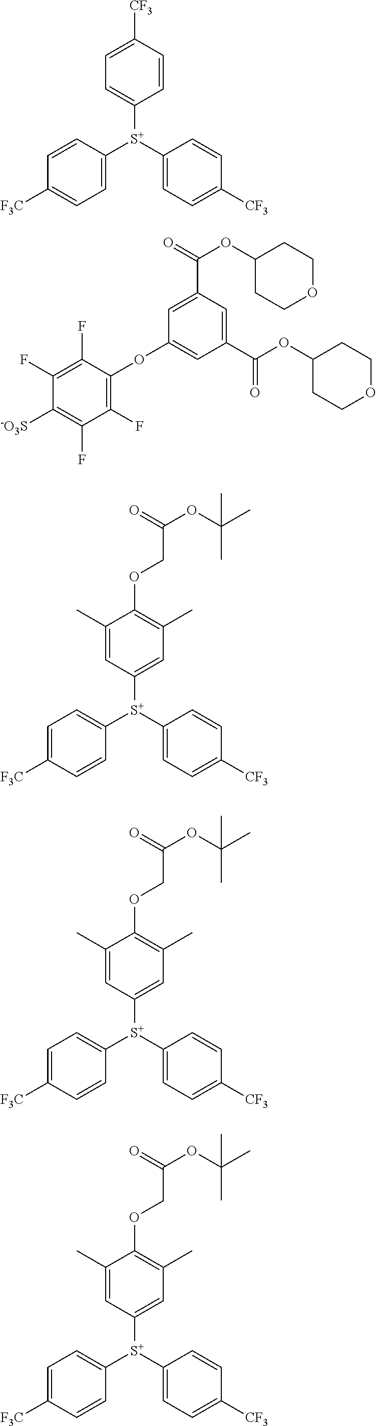

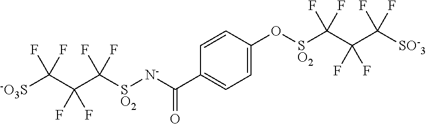

In the composition according to the embodiment of the present invention, a content of the photoacid generator (C) in the total solid content is preferably 0.1% to 20.0% by mass, more preferably 0.5% to 17.5% by mass, and still more preferably 1.0% to 15.0% by mass.

<Acid Diffusion Control Agent>

The composition according to the embodiment of the present invention may contain an acid diffusion control agent.

The acid diffusion control agent acts as a quencher which suppresses a reaction of an acid-decomposable resin in a non-exposed portion by excessive generated acids by trapping the acids generated from the photoacid generator and the like during exposure.

The type of the acid diffusion control agent is not particularly limited, and examples thereof include a basic compound (CA), a low-molecular-weight compound (CB) having a nitrogen atom and a group which is eliminated by action of acid, and a compound (CC) in which ability to control acid diffusion decreases or disappears in a case of being irradiated with actinic ray or radiation.

Examples of the compound (CC) include an onium salt compound (CD) which is a relatively weak acid with respect to the photoacid generator, and a basic compound (CE) in which basicity decreases or disappears in a case of being irradiated with actinic ray or radiation.

In addition, specific examples of the basic compound (CA) include compounds described in paragraphs [0132] to [0136] of WO2020/066824A; specific examples of the basic compound (CE) in which basicity decreases or disappears in a case of being irradiated with actinic ray or radiation include compounds described in paragraphs [0137] to [0155] of WO2020/066824A; specific examples of the low-molecular-weight compound (CB) having a nitrogen atom and a group which is eliminated by action of acid include paragraphs [0156] to [0163] of WO2020/066824A; and specific examples of the basic compound (CE) in which basicity decreases or disappears in a case of being irradiated with actinic ray or radiation include paragraph [0164] of WO2020/066824A.

In addition, specific examples of the onium salt compound (CD) which is a relatively weak acid with respect to the photoacid generator include compounds described in paragraphs [0305] to [0314] of WO2020/158337A.

In addition to above, for example, as the acid diffusion control agent, known compounds described in paragraphs [0627] to [0664] of US2016/0070167A1, paragraphs [0095] to [0187] of US2015/0004544A1, paragraphs [0403] to [0423] of US2016/0237190A1, and paragraphs [0259] to [0328] of US2016/0274458A1 can be suitably used.

In a case where the composition according to the embodiment of the present invention contains the acid diffusion control agent, a content of the acid diffusion control agent (in a case of a plurality of types, the total thereof) is preferably 0.1% to 15.0% by mass, and more preferably 0.5% to 15.0% by mass with respect to the total solid content of the composition. In the composition according to the embodiment of the present invention, the acid diffusion control agent may be used alone or in combination of two or more kinds thereof.

<Hydrophobic Resin>

The composition according to the embodiment of the present invention may further contain a hydrophobic resin different from the resin (A).

Although it is preferable that the hydrophobic resin is designed to be unevenly distributed on a surface of the resist film, it is not necessary to have a hydrophilic group in the molecule as different from a surfactant, and is not necessary to contribute to uniform mixing of polar materials and non-polar materials.

Examples of an effect caused by the addition of the hydrophobic resin include a control of static and dynamic contact angles of a surface of the resist film with respect to water and suppression of outgas.

From the viewpoint of uneven distribution on the film surface layer, the hydrophobic resin preferably has any one or more of a fluorine atom, a silicon atom, and a CH₃ partial structure which is included in a side chain moiety of a resin, and more preferably has two or more kinds thereof. In addition, the above-described hydrophobic resin preferably has a hydrocarbon group having 5 or more carbon atoms. These groups may be included in the main chain of the resin or may be substituted in the side chain of the resin.

Examples of the hydrophobic resin include compounds described in paragraphs [0275] to [0279] of WO2020/004306A.

In a case where the composition according to the embodiment of the present invention contains a hydrophobic resin, a content of the hydrophobic resin is preferably 0.01% to 20.0% by mass, and more preferably 0.1% to 15.0% by mass with respect to the total solid content of the composition.

<Surfactant>

The composition according to the embodiment of the present invention may contain a surfactant. In a case where the surfactant is included, it is possible to form a pattern having more excellent adhesiveness and fewer development defects.

The surfactant is preferably a fluorine-based and/or silicon-based surfactant.

Examples of the fluorine-based and/or silicon-based surfactant include the surfactants disclosed in paragraphs [0218] and [0219] of WO2018/193954A.

The surfactant may be used alone or in combination of two or more kinds thereof.

In a case where the composition according to the embodiment of the present invention contains a surfactant, a content of the surfactant is preferably 0.0001% to 2.0% by mass, more preferably 0.0005% to 1.0% by mass, and still more preferably 0.1% to 1.0% by mass with respect to the total solid content of the composition.

<Solvent>

The composition according to the embodiment of the present invention may contain a solvent.

The solvent preferably includes at least one solvent of (M1) propylene glycol monoalkyl ether carboxylate or (M2) at least one selected from the group consisting of propylene glycol monoalkyl ether, lactic acid ester, acetic acid ester, alkoxypropionic acid ester, chain ketone, cyclic ketone, lactone, and alkylene carbonate. The above-described solvent may further include a component other than the components (M1) and (M2).

The present inventors have found that, by using such a solvent and the above-described resin in combination, a pattern having a small number of development defects can be formed while improving coating property of the composition. A reason for this is not always clear, but the present inventors have considered that, since these solvents have a good balance of solubility, boiling point, and viscosity of the above-described resin, unevenness of a film thickness of a resist film, generation of precipitates during spin coating, and the like can be suppressed.

Details of the component (M1) and the component (M2) are described in paragraphs [0218] to [0226] of WO2020/004306A, the contents of which are incorporated herein by reference.

As described above, the solvent may further include a component other than the components (M1) and (M2). In this case, a content of the component other than the components (M1) and (M2) is preferably 5% to 30% by mass with respect to the total amount of the solvent.

A content of the solvent in the composition according to the embodiment of the present invention is preferably set such that the concentration of solid contents is 0.5% to 30% by mass, and more preferably set such that the concentration of solid contents is 1% to 20% by mass. With this content, the coating property of the composition according to the embodiment of the present invention can be further improved.

The solid content means all components other than the solvent, and as described above, it means components forming the actinic ray-sensitive or radiation-sensitive film.

The concentration of solid contents is a mass percentage of other components excluding the solvent with respect to the total mass of the composition according to the embodiment of the present invention.

The “total solid content” refers to a total mass of components obtained by removing the solvent from the whole composition of the composition according to the embodiment of the present invention. In addition, the “solid content” refers to components excluding the solvent as described above, and the components may be, for example, a solid or a liquid at 25° C.

<Other Additives>

The composition according to the embodiment of the present invention may further contain a dissolution inhibiting compound, a dye, a plasticizer, a photosensitizer, a light absorbing agent, and/or a compound promoting a solubility in a developer (for example, a phenol compound having a molecular weight of 1,000 or less or an alicyclic or aliphatic compound including a carboxyl group), or the like.

The composition according to the embodiment of the present invention may further contain a dissolution inhibiting compound. Here, the “dissolution inhibiting compound” is intended to be a compound having a molecular weight of 3000 or less, in which solubility in an organic developer decreases by decomposition due to action of acid.

The composition according to the embodiment of the present invention is suitably used as a composition for EUV light.

Since the EUV light has a wavelength of 13.5 nm and has a shorter wavelength than that of ArF (wavelength: 193 nm), the number of incident photons in a case of being exposed with the same sensitivity is small. Therefore, influence of “photon shot noise” in which the number of photons varies probabilistically is large, which causes deterioration of LER and bridge defects. In order to reduce the photon shot noise, there is a method of increasing the number of incident photons by increasing an exposure amount, but there is a trade-off with the demand for higher sensitivity.

In a case where an A value obtained by Expression (1) is high, absorption efficiency of EUV light and electron beams of the resist film formed from the composition according to the embodiment of the present invention is high, which is effective in reducing the photon shot noise. The A value represents the absorption efficiency of EUV light and electron beams of the resist film in terms of a mass proportion.

A=([H]×0.04+[C]×1.0+[N]×2.1+[O]×3.6+[F]×5.6+[S]×1.5+[I]×39.5)/([H]×1+[C]×12+[N]×14+[O]×16+[F]×19+[S]×32+[I]×127) Expression (1):

The A value is preferably 0.120 or more. The upper limit thereof is not particularly limited, but in a case where the A value is extremely high, the transmittance of EUV light and electron beams of the resist film is lowered and the optical image profile in the resist film is deteriorated, which results in difficulty in obtaining a good pattern shape, and therefore, the upper limit is preferably 0.240 or less, and more preferably 0.220 or less.

In Expression (1), [H] represents a molar ratio of hydrogen atoms derived from a total solid content with respect to all atoms of the total solid content in the actinic ray-sensitive or radiation-sensitive resin composition, [C] represents a molar ratio of carbon atoms derived from the total solid content with respect to all atoms of the total solid content in the actinic ray-sensitive or radiation-sensitive resin composition, [N] represents a molar ratio of nitrogen atoms derived from the total solid content with respect to all atoms of the total solid content in the actinic ray-sensitive or radiation-sensitive resin composition, [O] represents a molar ratio of oxygen atoms derived from the total solid content with respect to all atoms of the total solid content in the actinic ray-sensitive or radiation-sensitive resin composition, [F] represents a molar ratio of fluorine atoms derived from the total solid content with respect to all atoms of the total solid content in the actinic ray-sensitive or radiation-sensitive resin composition, [S]represents a molar ratio of sulfur atoms derived from the total solid content with respect to all atoms of the total solid content in the actinic ray-sensitive or radiation-sensitive resin composition, and [I] represents a molar ratio of iodine atoms derived from the total solid content with respect to all atoms of the total solid content in the actinic ray-sensitive or radiation-sensitive resin composition.

For example, in a case where the resist composition contains an acid-decomposable resin, a photoacid generator, an acid diffusion control agent, and a solvent, the acid-decomposable resin, the photoacid generator, and the acid diffusion control agent correspond to the solid content. That is, all the atoms of the total solid content correspond to a sum of all the atoms derived from the resin, all the atoms derived from the photoacid generator, and all the atoms derived from the acid diffusion control agent. For example, [H] represents a molar ratio of hydrogen atoms derived from the total solid content with respect to all the atoms in the total solid content, and by way of description based on the example above, [H] represents a molar ratio of a sum of the hydrogen atoms derived from the acid-decomposable resin, the hydrogen atoms derived from the photoacid generator, and the hydrogen atoms derived from the acid diffusion control agent with respect to a sum of all the atoms derived from the acid-decomposable resin, all the atoms derived from the photoacid generator, and all the atoms derived from the acid diffusion control agent.

The A value can be calculated by computation of the structure of constituent components of the total solid content in the resist composition, and the ratio of the number of atoms contained in a case where the content is already known. In addition, even in a case where the constituent component is not known yet, it is possible to calculate a ratio of the number of constituent atoms by subjecting a resist film obtained after evaporating the solvent components of the resist composition to computation according to an analytic approach such as elemental analysis.

In addition, the present invention also relates to an actinic ray-sensitive or radiation-sensitive resin composition containing (A) a resin which is decomposed by action of acid to increase polarity and (B) a compound which generates an acid by irradiation with an actinic ray or a radiation, in which the resin (A) is a resin having an acid group, an alcoholic hydroxyl group, or an acid-decomposable group, and the compound (B) is an ionic compound having a partial structure represented by any of General Formula (1), (2), or (3) in an anionic moiety.

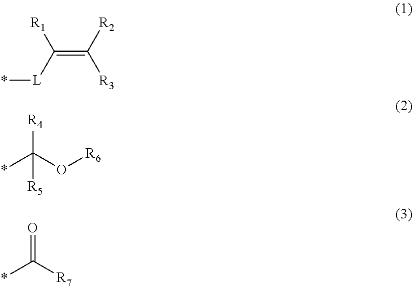

In General Formula (1), R₁ to R₃ each independently represent a hydrogen atom or a substituent, L represents a single bond or a divalent linking group, and * represents a bonding position.

In General Formula (2), R₄ to R₆ each independently represent a hydrogen atom or a substituent, and * represents a bonding position.

In General Formula (3), R₇ represents a hydrogen atom or a substituent, and * represents a bonding position.

In the above-described resin (A), each of the acid group, the alcoholic hydroxyl group, or the acid-decomposable group is as described above.

Examples of the above-described resin (A) include the resin (A) described above.

Each group in the above-described partial structure represented by any of General Formula (1), (2), or (3) is as described above.

Examples of the above-described compound (B) include the compound (B) described above.

The above-described composition is one aspect for achieving the above-described actinic ray-sensitive or radiation-sensitive resin composition containing (A) a resin which is decomposed by action of acid to increase polarity and (B) a compound which generates an acid by irradiation with an actinic ray or a radiation, in which the resin (A) and the acid generated from the compound (B) form a bond by the actinic ray or the radiation or by the action of acid.

[Use]

The composition according to the embodiment of the present invention relates to an actinic ray-sensitive or radiation-sensitive resin composition having properties which change by undergoing a reaction by irradiation with actinic ray or radiation. More specifically, the composition according to the embodiment of the present invention relates to an actinic ray-sensitive or radiation-sensitive resin composition which is used in a step of manufacturing a semiconductor such as an integrated circuit (IC), for manufacturing of a circuit board for a liquid crystal, a thermal head, or the like, manufacturing of a mold structure for imprinting, other photofabrication steps, or production of a planographic printing plate or an acid-curable composition. A pattern formed in the present invention can be used in an etching step, an ion implanting step, a bump electrode forming step, a rewiring forming step, a microelectromechanical system (MEMS), or the like.

[Actinic Ray-Sensitive or Radiation-Sensitive Film]

The present invention also relates to an actinic ray-sensitive or radiation-sensitive film (typically, “resist film”) formed of the actinic ray-sensitive or radiation-sensitive composition according to the present invention. Such a film is formed, for example, by applying the composition according to the embodiment of the present invention onto a support such as a substrate. A thickness of the film is preferably 0.01 to 0.15 μm.

As a method for applying the composition onto the substrate, the composition is applied to the substrate by an appropriate coating method such as a spin coating, a roll coating, a flow coating, a dip coating, a spray coating, and a doctor coating. Among these, a spin coating is preferable, and the rotation speed thereof is preferably 1000 to 3000 rotations per minute (rpm). The coating film is prebaked at 60° C. to 150° C. for 1 to 20 minutes, preferably at 80° C. to 120° C. for 1 to 10 minutes to form a thin film.

For a material constituting the substrate to be processed and an outermost layer thereof, for example, in a case of a semiconductor wafer, a silicon wafer can be used, and examples of the material forming the outermost layer include Si, SiO₂, SiN, SiON, and TiN, WSi, BPSG, SOG, and an organic antireflection film.

[Pattern Forming Method]

A procedure of a pattern forming method using the above-described actinic ray-sensitive or radiation-sensitive resin composition is not particularly limited, but it is preferable to include the following steps.

Step 1: step of forming an actinic ray-sensitive or radiation-sensitive film on a substrate using the actinic ray-sensitive or radiation-sensitive resin composition

Step 2: step of exposing the actinic ray-sensitive or radiation-sensitive film

Step 3: step of developing the exposed actinic ray-sensitive or radiation-sensitive film with a developer to form a pattern Hereinafter, the procedure of each of the above-described steps will be described in detail.

<Step 1: Actinic Ray-Sensitive or Radiation-Sensitive Film Forming Step>

The step 1 is a step of forming an actinic ray-sensitive or radiation-sensitive film on a substrate using the actinic ray-sensitive or radiation-sensitive resin composition.

Examples of the method of forming the actinic ray-sensitive or radiation-sensitive film on the substrate using the actinic ray-sensitive or radiation-sensitive resin composition include a method of applying the actinic ray-sensitive or radiation-sensitive resin composition onto the substrate.

In addition, it is preferable that the actinic ray-sensitive or radiation-sensitive resin composition before the application is filtered through a filter, as desired. A pore size of the filter is preferably 0.1 μm or less, more preferably 0.05 μm or less, and still more preferably 0.03 μm or less. In addition, the filter is preferably a polytetrafluoroethylene-made filter, a polyethylene-made filter, or a nylon-made filter.

The actinic ray-sensitive or radiation-sensitive resin composition can be applied onto the substrate (for example, silicon and silicon dioxide coating) as used in the manufacture of integrated circuit elements by a suitable application method such as an application using a spinner or a coater. The coating method is preferably a spin coating using a spinner. A rotation speed upon the spin application using a spinner is preferably 1000 to 3000 rpm.

After the application of the actinic ray-sensitive or radiation-sensitive resin composition, the substrate may be dried to form the resist film. In addition, various underlying films (an inorganic film, an organic film, or an antireflection film) may be formed on an underlayer of the resist film.

Examples of the drying method include a method of heating and drying. The heating can be carried out using a unit included in an ordinary exposure machine and/or development machine, and may also be carried out using a hot plate or the like. A heating temperature is preferably 80° C. to 150° C., more preferably 80° C. to 140° C., and still more preferably 80° C. to 130° C. A heating time is preferably 30 to 1000 seconds, more preferably 60 to 800 seconds, and still more preferably 60 to 600 seconds.

A film thickness of the actinic ray-sensitive or radiation-sensitive film is not particularly limited, but from the viewpoint that a fine pattern having higher accuracy can be formed, is preferably 10 to 120 nm.

Among these, in a case of performing EUV exposure, the film thickness of the actinic ray-sensitive or radiation-sensitive film is more preferably 10 to 65 nm and still more preferably 15 to 50 nm. In addition, in a case of performing ArF liquid immersion exposure, the film thickness of the actinic ray-sensitive or radiation-sensitive film is more preferably 10 to 120 nm and still more preferably 15 to 90 nm.

A topcoat may be formed on an upper layer of the actinic ray-sensitive or radiation-sensitive film using a topcoat composition.

It is preferable that the topcoat composition is not mixed with the actinic ray-sensitive or radiation-sensitive film and can be uniformly applied onto the upper layer of the actinic ray-sensitive or radiation-sensitive film.

The topcoat is not particularly limited, a topcoat known in the related art can be formed by the methods known in the related art, and for example, the topcoat can be formed based on the description in paragraphs [0072] to [0082] of JP2014-059543A.

For example, it is preferable that a topcoat including a basic compound as described in JP2013-61648A is formed on the actinic ray-sensitive or radiation-sensitive film. Specific examples of the basic compound which can be included in the topcoat include a basic compound which may be contained in the actinic ray-sensitive or radiation-sensitive resin composition described above.

In addition, it is also preferable that the topcoat includes a compound which includes at least one group or bond selected from the group consisting of an ether bond, a thioether bond, a hydroxyl group, a thiol group, a carbonyl bond, and an ester bond.

<Step 2: Exposing Step>

The step 2 is a step of exposing the actinic ray-sensitive or radiation-sensitive film.

Examples of an exposing method include a method in which the formed actinic ray-sensitive or radiation-sensitive film is irradiated with actinic ray-sensitive or radiation through a predetermined mask.

Examples of the actinic ray or radiation include infrared light, visible light, ultraviolet light, far ultraviolet light, extreme ultraviolet light, X-rays, and electron beams, preferably a far ultraviolet light having a wavelength of 250 nm or less, more preferably a far ultraviolet light having a wavelength of 220 nm or less, and particularly preferably a far ultraviolet light having a wavelength of 1 to 200 nm, specifically, KrF excimer laser (248 nm), ArF excimer laser (193 nm), F₂ excimer laser (157 nm), EUV (13 nm), X-rays, and electron beams.

It is preferable to perform baking (heating) before performing development and after the exposure. The baking accelerates a reaction in the exposed portion, and the sensitivity and the pattern shape are improved.

A heating temperature is preferably 80° C. to 150° C., more preferably 80° C. to 140° C., and still more preferably 80° C. to 130° C.

A heating time is preferably 10 to 1000 seconds, more preferably 10 to 180 seconds, and still more preferably 30 to 120 seconds.

The heating can be carried out using a unit included in an ordinary exposure machine and/or development machine, and may also be performed using a hot plate or the like.

This step is also referred to as a post-exposure baking.

<Step 3: Developing Step>

The step 3 is a step of developing the exposed actinic ray-sensitive or radiation-sensitive film with a developer to form a pattern.

The developer may be either an alkali developer or a developer containing an organic solvent (hereinafter, also referred to as an organic developer).

Examples of a developing method include a method in which the substrate is immersed in a tank filled with a developer for a certain period of time (a dipping method), a method in which a development is performed by heaping a developer up onto the surface of the substrate by surface tension, and then leaving it to stand for a certain period of time (a puddle method), a method in which a developer is sprayed on the surface of the substrate (a spraying method), and a method in which a developer is continuously jetted onto the substrate rotating at a constant rate while scanning a developer jetting nozzle at a constant rate (a dynamic dispensing method).

In addition, after the step of performing the development, a step of stopping the development may be carried out while replacing the solvent with another solvent.

A developing time is not particularly limited as long as it is a period of time where the non-exposed portion of the resin is sufficiently dissolved, and is preferably 10 to 300 seconds and more preferably 20 to 120 seconds.

A temperature of the developer is preferably 0° C. to 50° C. and more preferably 15° C. to 35° C.

As the alkali developer, it is preferable to use an aqueous alkali solution including an alkali. The type of the aqueous alkali solution is not particularly limited, and examples thereof include an aqueous alkali solution including a quaternary ammonium salt typified by tetramethylammonium hydroxide, an inorganic alkali, a primary amine, a secondary amine, a tertiary amine, an alcoholamine, a cyclic amine, or the like. Among these, the alkali developer is preferably aqueous solutions of the quaternary ammonium salts typified by tetramethylammonium hydroxide (TMAH). An appropriate amount of alcohols, a surfactant, or the like may be added to the alkali developer. An alkali concentration of the alkali developer is usually 0.1% to 20% by mass. In addition, a pH of the alkali developer is usually 10.0 to 15.0.

The organic developer is preferably a developer containing at least one organic solvent selected from the group consisting of a ketone-based solvent, an ester-based solvent, an alcohol-based solvent, an amide-based solvent, an ether-based solvent, and a hydrocarbon-based solvent.

A plurality of the above-described solvents may be mixed, or the solvent may be used in admixture with a solvent other than those described above or water. A moisture content in the entire developer is preferably less than 50% by mass, more preferably less than 20% by mass, and still more preferably less than 10% by mass, and it is particularly preferable that the entire developer contains substantially no water.

A content of the organic solvent with respect to the organic developer is preferably 50% by mass to 100% by mass, more preferably 80% by mass to 100% by mass, still more preferably 90% by mass to 100% by mass, and particularly preferably 95% by mass to 100% by mass with respect to the total amount of the developer.

<Other Steps>

It is preferable that the above-described pattern forming method includes a step of performing washing using a rinsing liquid after the step 3.

Examples of the rinsing liquid used in the rinsing step after the step of performing development using an alkali developer include pure water. An appropriate amount of a surfactant may be added to the pure water. An appropriate amount of a surfactant may be added to the rinsing liquid.

The rinsing liquid used in the rinsing step after the developing step with an organic developer is not particularly limited as long as the rinsing liquid does not dissolve the pattern, and a solution including a common organic solvent can be used. As the rinsing liquid, a rinsing liquid containing at least one organic solvent selected from the group consisting of a hydrocarbon-based solvent, a ketone-based solvent, an ester-based solvent, an alcohol-based solvent, an amide-based solvent, and an ether-based solvent is preferably used.

A method for the rinsing step is not particularly limited, and examples thereof include a method in which the rinsing liquid is continuously jetted onto the substrate rotated at a constant rate (a spin coating method), a method in which the substrate is immersed in a tank filled with the rinsing liquid for a certain period of time (a dipping method), and a method in which the rinsing liquid is sprayed on the surface of the substrate (a spraying method).

In addition, the pattern forming method according to the embodiment of the present invention may include a heating step (postbaking) after the rinsing step. By this step, the developer and the rinsing liquid remaining between and inside the patterns are removed by baking. In addition, this step also has an effect that a resist pattern is annealed and the surface roughness of the pattern is improved. The heating step after the rinsing step is usually performed at 40° C. to 250° C. (preferably 90° C. to 200° C.) for usually 10 seconds to 3 minutes (preferably 30 seconds to 120 seconds).

In addition, an etching treatment on the substrate may be carried out using the formed pattern as a mask. That is, the substrate (or the underlayer film and the substrate) may be processed using the pattern formed in the step 3 as a mask to form a pattern on the substrate.

A method for processing the substrate (or the underlayer film and the substrate) is not particularly limited, but a method in which a pattern is formed on a substrate by subjecting the substrate (or the underlayer film and the substrate) to dry etching using the pattern formed in the step 3 as a mask is preferable. Oxygen plasma etching is preferable as the dry etching.

It is preferable that various materials (for example, the solvent, the developer, the rinsing liquid, a composition for forming the antireflection film, a composition for forming the topcoat, and the like) used in the composition according to the embodiment of the present invention and the pattern forming method according to the embodiment of the present invention do not include impurities such as metals. A content of the impurities included in these materials is preferably 1 ppm by mass or less, more preferably 10 ppb by mass or less, still more preferably 100 ppt by mass or less, particularly preferably 10 ppt by mass or less, and most preferably 1 ppt by mass or less. The lower limit thereof is not particularly limited, but is preferably 0 ppt by mass or more. Here, examples of the metal impurities include Na, K, Ca, Fe, Cu, Mg, Al, Li, Cr, Ni, Sn, Ag, As, Au, Ba, Cd, Co, Pb, Ti, V, W, and Zn.

Examples of a method for removing the impurities such as metals from the various materials include filtration using a filter. Details of the filtration using a filter are described in paragraph [0321] of WO2020/004306A.

In addition, examples of a method for reducing the impurities such as metals included in the various materials include a method of selecting raw materials having a low content of metals as raw materials constituting the various materials, a method of subjecting raw materials constituting the various materials to filter filtration, and a method of performing distillation under the condition for suppressing the contamination as much as possible by, for example, lining the inside of a device with TEFLON (registered trademark).

In addition to the filter filtration, removal of the impurities by an adsorbing material may be performed, or a combination of filter filtration and an adsorbing material may be used. As the adsorbing material, known adsorbing materials can be used, and for example, inorganic adsorbing materials such as silica gel and zeolite and organic adsorbing materials such as activated carbon can be used. It is necessary to prevent the incorporation of impurities such as metals in the production process in order to reduce the metal impurities included in the above-described various materials. Sufficient removal of the metal impurities from a production device can be confirmed by measuring the content of metal components included in a washing solution used to wash the production device. A content of the metal components included in the washing solution after the use is preferably 100 parts per trillion (ppt) by mass or less, more preferably 10 ppt by mass or less, and still more preferably 1 ppt by mass or less. The lower limit thereof is not particularly limited, but is preferably 0 ppt by mass or more.

A conductive compound may be added to an organic treatment liquid such as the rinsing liquid in order to prevent breakdown of chemical liquid pipes and various parts (a filter, an O-ring, a tube, and the like) due to electrostatic charging, and subsequently generated electrostatic discharging. The conductive compound is not particularly limited, and examples thereof include methanol. An addition amount is not particularly limited, but from the viewpoint that preferred development characteristics or rinsing characteristics are maintained, the addition amount is preferably 10% by mass or less and more preferably 5% by mass or less. The lower limit thereof is not particularly limited, but is preferably 0.01% by mass or more.

For members of the chemical liquid pipe, for example, various pipes coated with stainless steel (SUS), or a polyethylene, polypropylene, or a fluororesin (a polytetrafluoroethylene resin, a perfluoroalkoxy resin, or the like) that has been subjected to an antistatic treatment can be used. In the same manner, for the filter or the O-ring, polyethylene, polypropylene, or a fluororesin (polytetrafluoroethylene, a perfluoroalkoxy resin, or the like) that has been subjected to an antistatic treatment can be used.

<Method for Manufacturing Electronic Device>

In addition, the present invention further relates to a method for manufacturing an electronic device, including the above-described pattern forming method, and an electronic device manufactured by this manufacturing method.

Examples of a suitable aspect of the electronic device according to the embodiment of the present invention include an aspect of being mounted on electric and electronic apparatus (for example, home appliances, office automation (OA)-related equipment, media-related equipment, optical equipment, telecommunication equipment, and the like).

### EXAMPLES

Hereinbelow, the present invention will be described in more detail with reference to Examples. The materials, the amounts of materials used, the proportions, the treatment details, the treatment procedure, and the like shown in Examples below may be modified as appropriate as long as the modifications do not depart from the spirit of the present invention. Therefore, the scope of the present invention should not be construed as being limited to Examples shown below.

[Various Components of Actinic Ray-Sensitive or Radiation-Sensitive Resin Composition]

[Resin (A)]

Resins A (resins A-1 to A-25) shown in Table 3 are shown below.

As the resins A-1 to A-25, those synthesized according to a method for synthesizing a resin A-1 (Synthesis Example 1) which will be described later were used. A compositional ratio (ratio in % by mole) of each repeating unit shown below, a weight-average molecular weight (Mw), and a dispersity (Mw/Mn) are shown in Table 1.

The weight-average molecular weight (Mw) and the dispersity (Mw/Mn) of the resins A-1 to A-25 were measured by GPC (solvent: tetrahydrofuran (THF)). In addition, the compositional ratio (ratio in % by mole) of the resin was measured by means of ¹³C-nuclear magnetic resonance (NMR).

TABLE 1

| | Molar ratio of | | Molar ratio of | | Molar ratio of | | Molar ratio of | | | |
|---|---|---|---|---|---|---|---|---|---|---|
| | repeating unit 1 | | repeating unit 2 | | repeating unit 3 | | repeating unit 4 | | Mw | Mw/Mn |
| Resin A-1 | MB-10 | 50 | MA-16 | 50 | | | | | 8500 | 1.60 |
| Resin A-2 | MB-15 | 40 | MA-7 | 60 | | | | | 9000 | 1.70 |
| Resin A-3 | MB-7 | 30 | MB-14 | 10 | MA-6 | 60 | | | 7000 | 1.55 |
| Resin A-4 | MB-5 | 50 | MB-12 | 10 | MA-15 | 40 | | | 7500 | 1.55 |
| Resin A-5 | MB-6 | 30 | MB-19 | 10 | MA-3 | 30 | MA-4 | 30 | 9500 | 1.45 |
| Resin A-6 | MB-9 | 30 | MB-20 | 10 | MA-13 | 60 | | | 12000 | 1.65 |
| Resin A-7 | MB-19 | 20 | MB-4 | 20 | MA-2 | 60 | | | 6000 | 1.55 |
| Resin A-8 | MB-16 | 30 | MA-17 | 70 | | | | | 8000 | 1.40 |
| Resin A-9 | MB-30 | 20 | MA-8 | 80 | | | | | 5500 | 1.65 |
| Resin A-10 | MB-13 | 50 | MA-10 | 50 | | | | | 15000 | 1.75 |
| Resin A-11 | MB-8 | 30 | MA-20 | 70 | | | | | 9000 | 1.60 |
| Resin A-12 | MB-18 | 30 | MB-29 | 30 | MA-14 | 40 | | | 8000 | 1.55 |
| Resin A-13 | MB-1 | 30 | MB-26 | 30 | MA-11 | 40 | | | 18000 | 1.80 |
| Resin A-14 | MB-27 | 60 | MA-5 | 40 | | | | | 7500 | 1.65 |
| Resin A-15 | MB-17 | 30 | MB-24 | 10 | MA-19 | 60 | | | 8000 | 1.70 |
| Resin A-16 | MB-11 | 30 | MB-28 | 40 | MA-9 | 30 | | | 9500 | 1.80 |
| Resin A-17 | MB-2 | 60 | MA-1 | 40 | | | | | 11000 | 1.65 |
| Resin A-18 | MB-19 | 20 | MB-21 | 20 | MA-2 | 30 | MA-8 | 30 | 6500 | 1.60 |
| Resin A-19 | MB-23 | 40 | MA-10 | 40 | MA-18 | 10 | MA-20 | 10 | 8000 | 1.55 |
| Resin A-20 | MB-25 | 30 | MA-2 | 70 | | | | | 7500 | 1.60 |
| Resin A-21 | MB-3 | 40 | MA-3 | 60 | | | | | 9500 | 1.60 |
| Resin A-22 | MB-22 | 40 | MA-4 | 60 | | | | | 10000 | 1.70 |
| Resin A-23 | MB-19 | 15 | MB-21 | 15 | MA-2 | 70 | | | 7500 | 1.60 |
| Resin A-24 | MB-19 | 40 | MA-2 | 30 | MA-8 | 30 | | | 8000 | 1.65 |
| Resin A-25 | MB-31 | 50 | MA-12 | 50 | | | | | 6500 | 1.60 |

Structures of monomers MA-1 to MA-20 and monomers MB-1 to MB-31 corresponding to respective repeating units constituting the resins A-1 to A-25 shown in Table 1 are shown below.

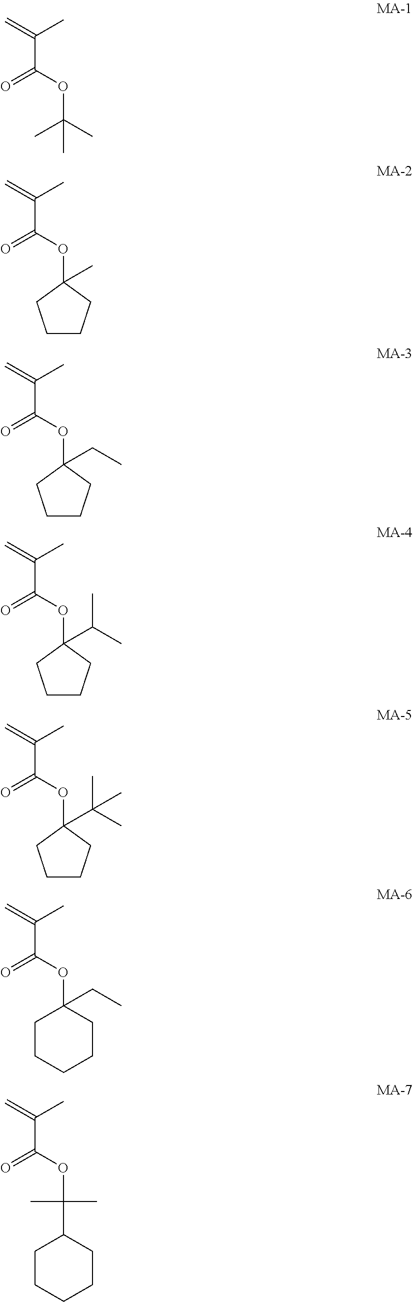

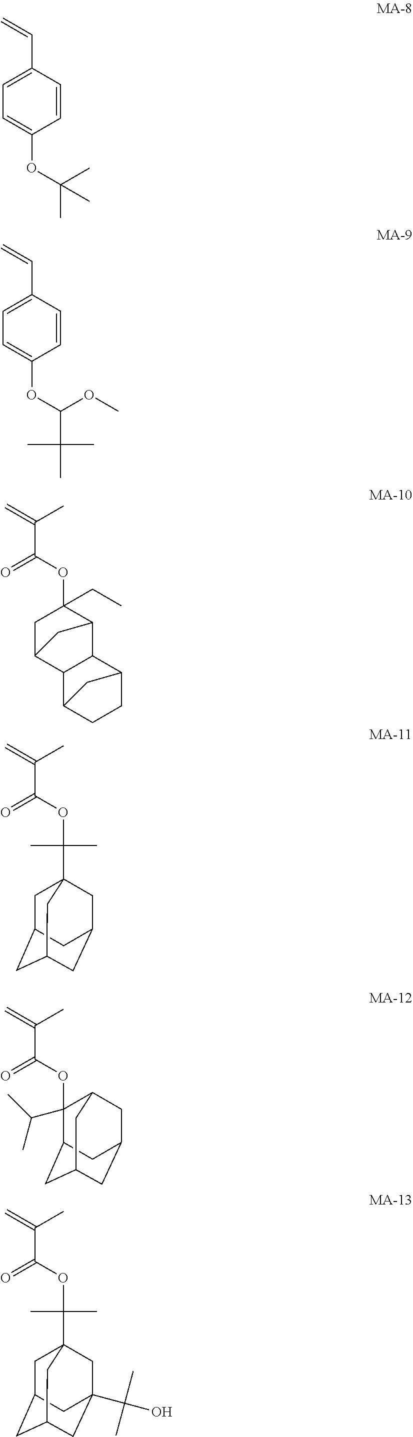

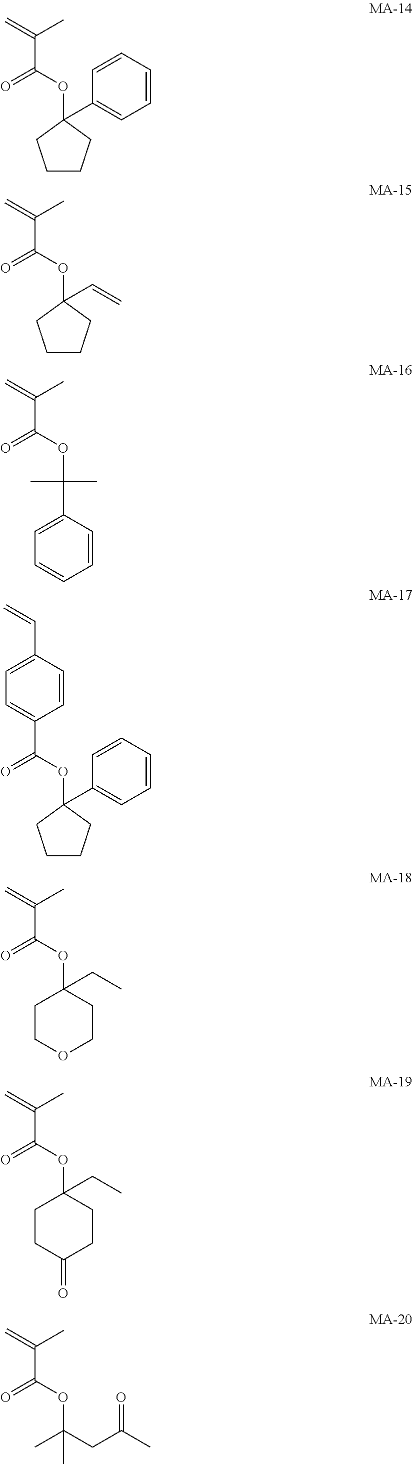

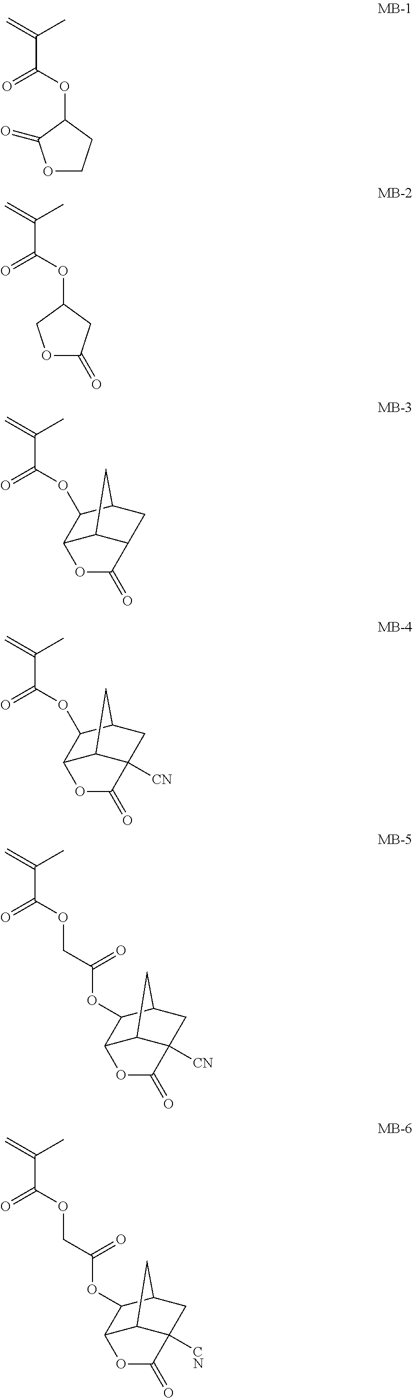

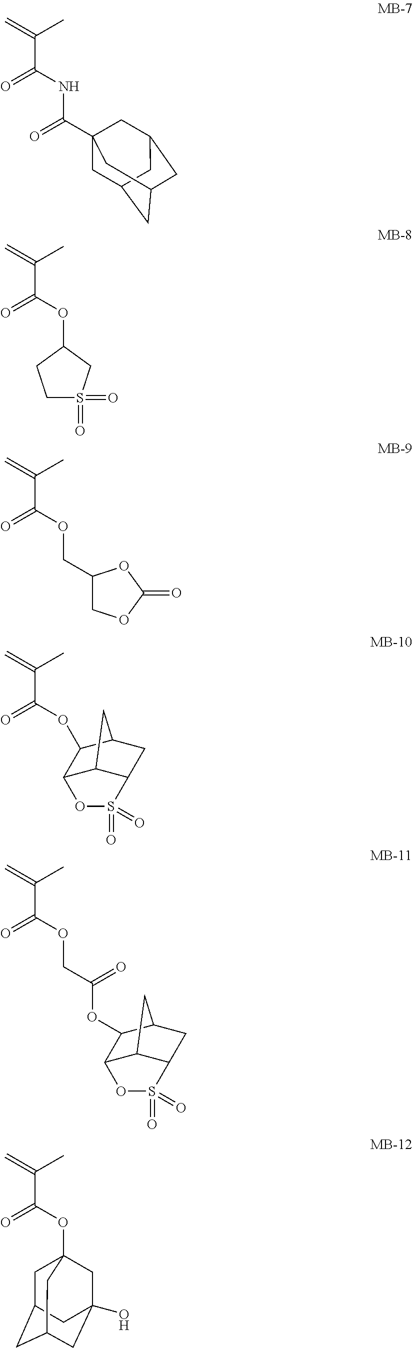

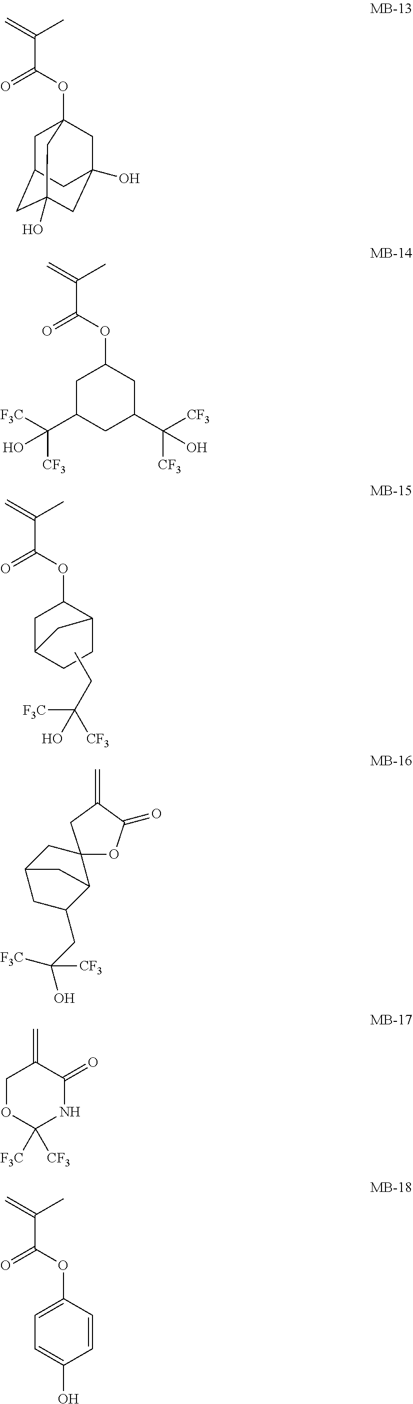

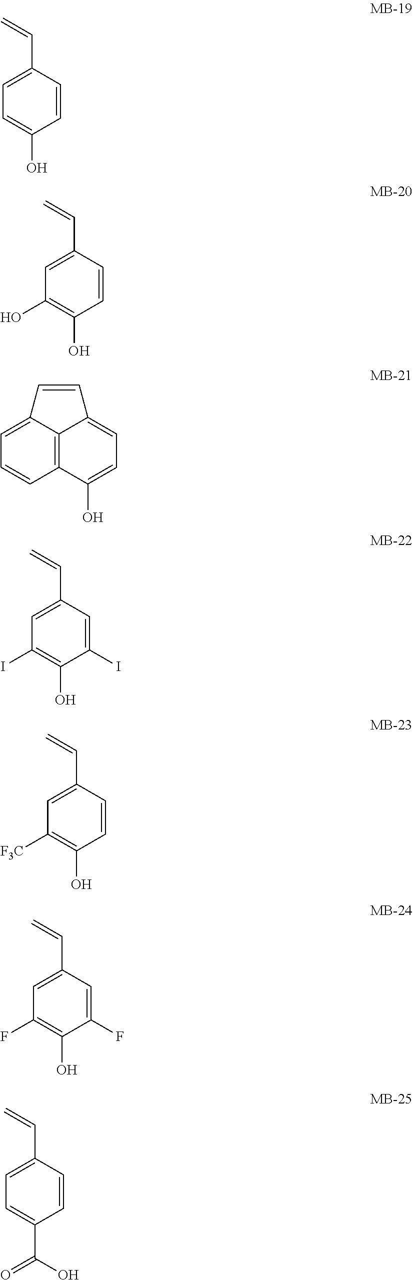

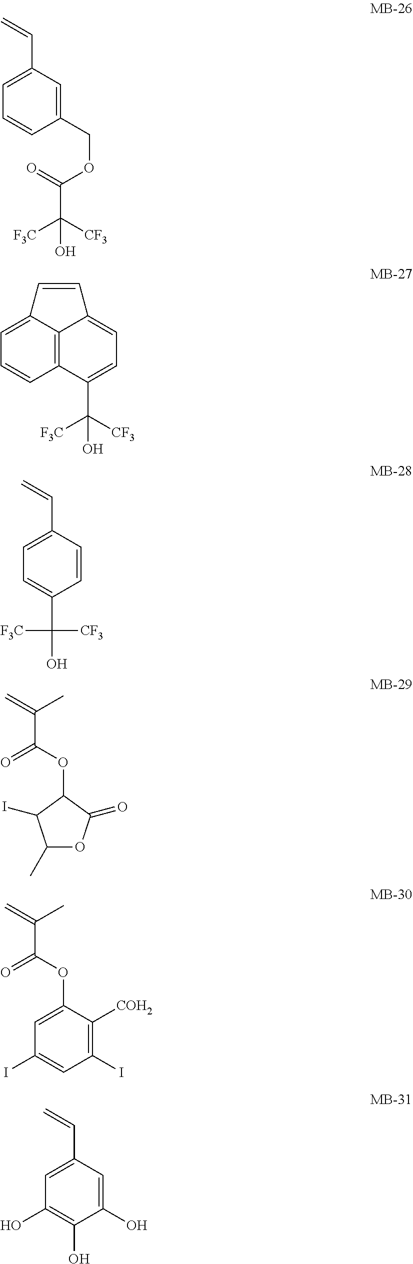

### Synthesis Example 1: Synthesis of Resin A-1

61 parts by mass of cyclohexanone was heated to 85° C. under a nitrogen stream. A mixed solution of 21 parts by mass of a monomer represented by Structural formula MB-10, 16 parts by mass of a monomer represented by Structural Formula MA-16, 60 parts by mass of cyclohexanone, and 3 parts by mass of dimethyl 2,2′-azobisisobutyrate [V-601, manufactured by FUJIFILM Wako Pure Chemical Corporation] was added dropwise to the liquid over 6 hours under stirring to obtain a reaction solution. After completion of the dropwise addition, the reaction solution was further stirred at 85° C. for 2 hours. The reaction solution was cooled, then reprecipitated with a large amount of methanol/water (mass ratio: 9:1), and filtered, and the obtained solid was vacuum-dried to obtain a resin A-1.

The obtained resin A-1 had a weight-average molecular weight (Mw; in terms of polystyrene) of 8500 and a dispersity (Mw/Mn) of 1.6, as determined from GPC (carrier: tetrahydrofuran (THF)). The compositional ratio of the repeating unit derived from MB-10 and the repeating unit derived from MA-16, which was measured by ¹³C-nuclear magnetic resonance (NMR), was 50/50 in a molar ratio.

Other resins were also synthesized in the same manner.

[Photoacid Generator]

<Photoacid Generator (B)>

Structures of photoacid generators (B) (compounds B-1 to B-12) shown in Table 3 are shown below.

As the compounds B-1 to B-12, those synthesized according to a method for synthesizing a compound B-1 (Synthesis Example 2) which will be described later were used.

Although the compound B-9 was not the photoacid generator (B), it is listed in Table 3 for convenience.

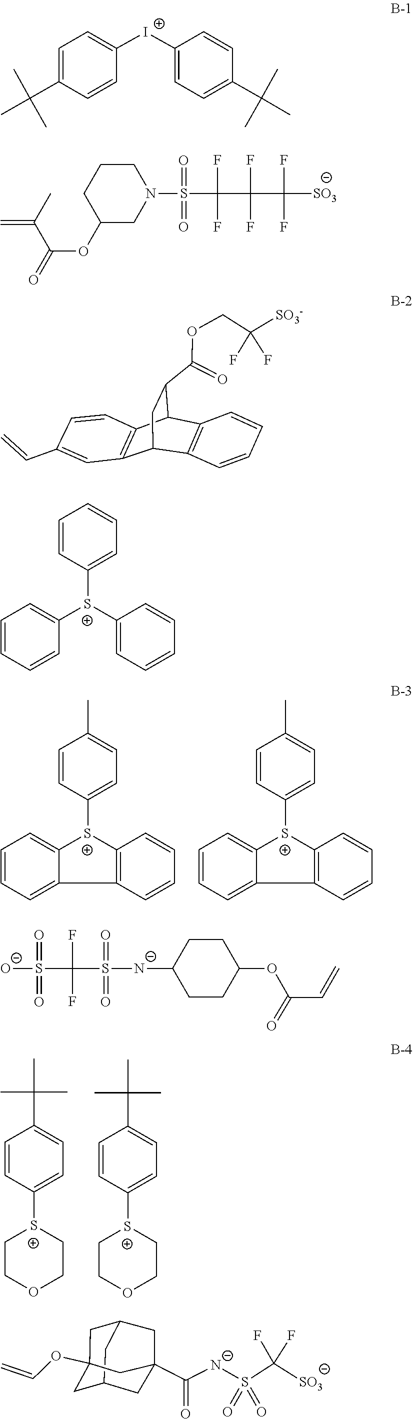

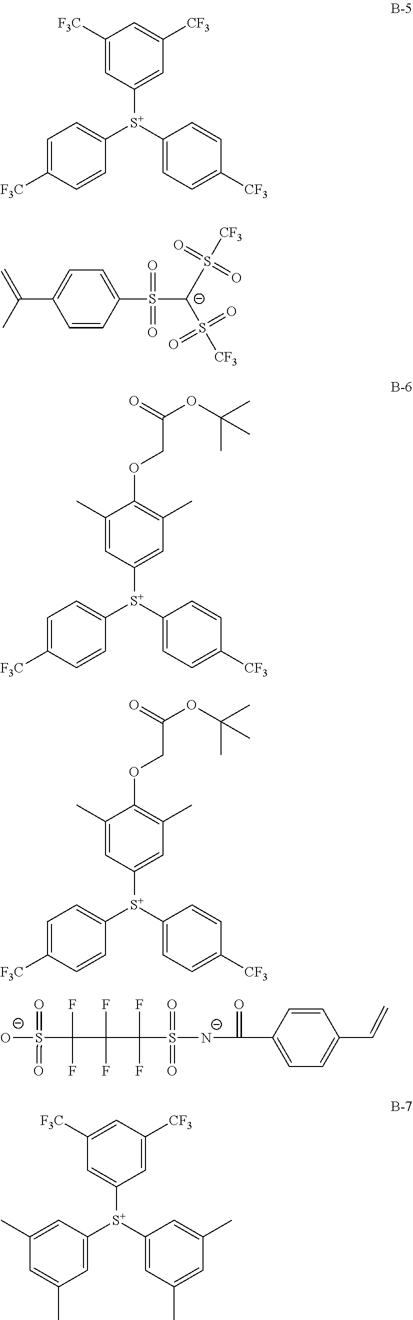

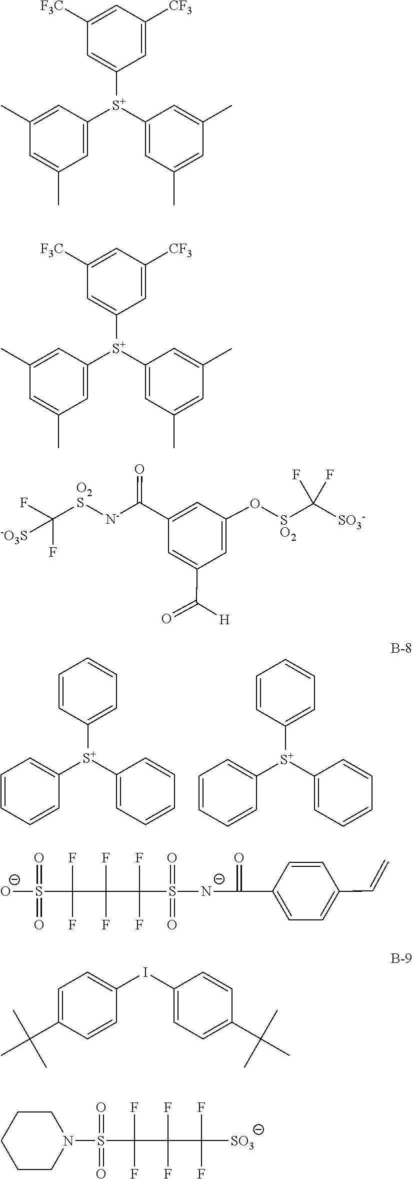

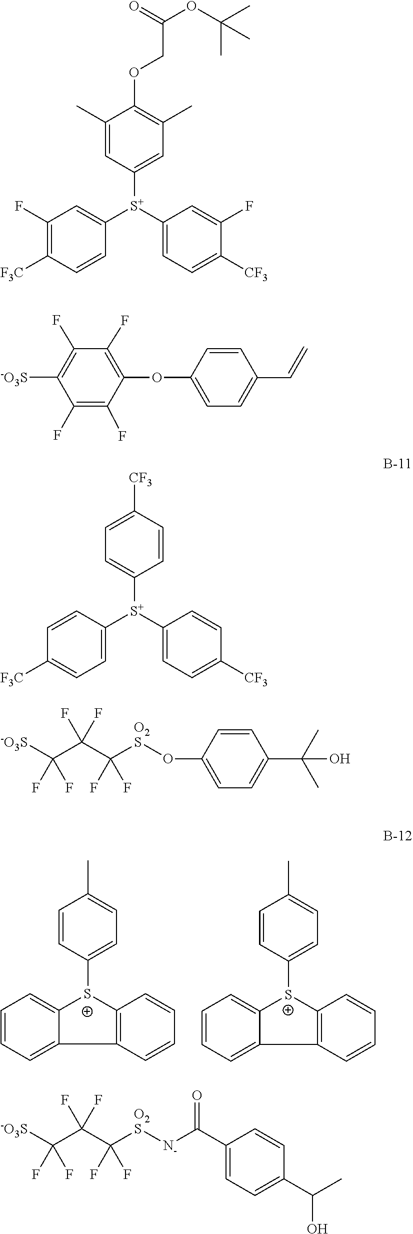

### Synthesis Example 2: Synthesis of Compound B-1

Methylene chloride (100 mL) and water (100 mL) were mixed to obtain a mixed solution. B-1-a (10.0 g) and B-1-b (8.9 g) were added to the above-described mixed solution. The above-described mixed solution was stirred for 1 hour, and then the water phase was removed from the above-described mixed solution. The remaining organic phase was washed with 1% by mass potassium carbonate aqueous solution (100 mL), 0.01 N hydrochloric acid (100 mL), and water (100 mL). The solvent was distilled off from the above-described organic phase to obtain B-1 (15.5 g, yield: 99%).

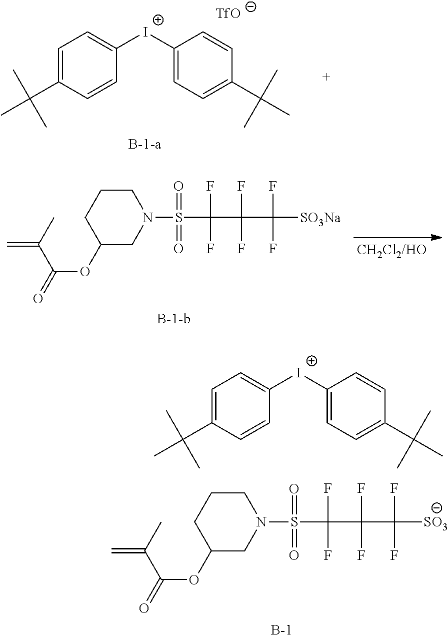

Other photoacid generators were synthesized with reference to the above-described synthesis method.

<Photoacid Generator (C)>

Structures of photoacid generators (C) (compounds C-1 to C-11) shown in Table 3 are shown below.

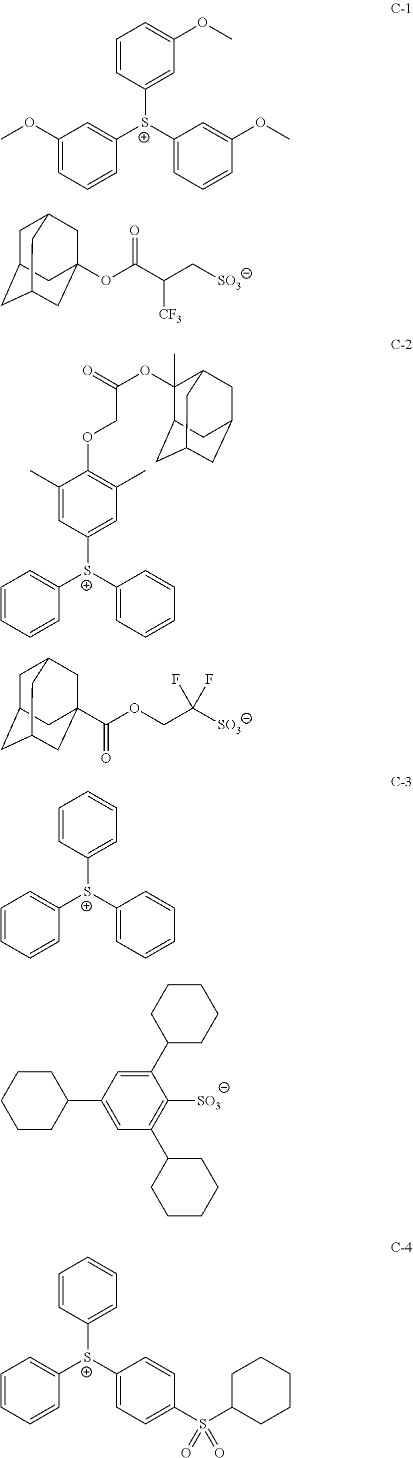

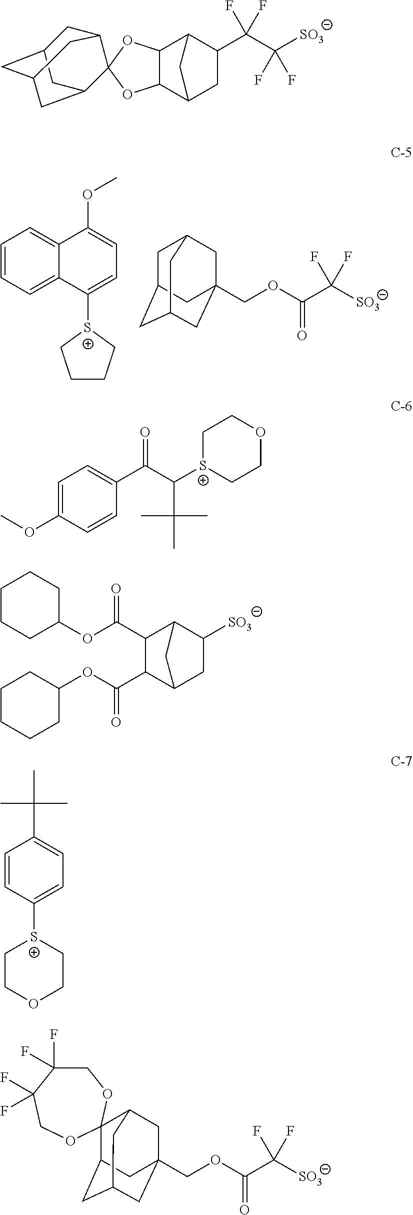

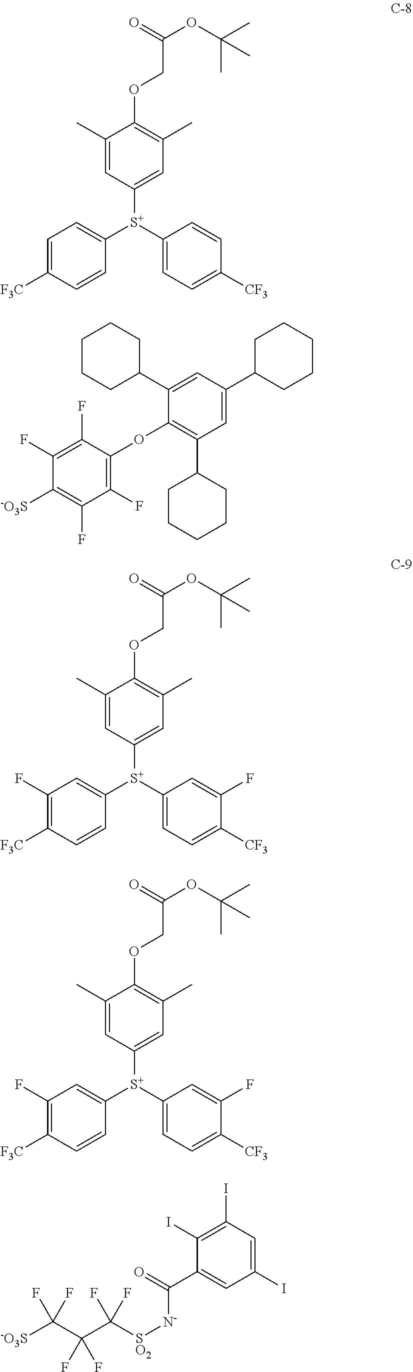

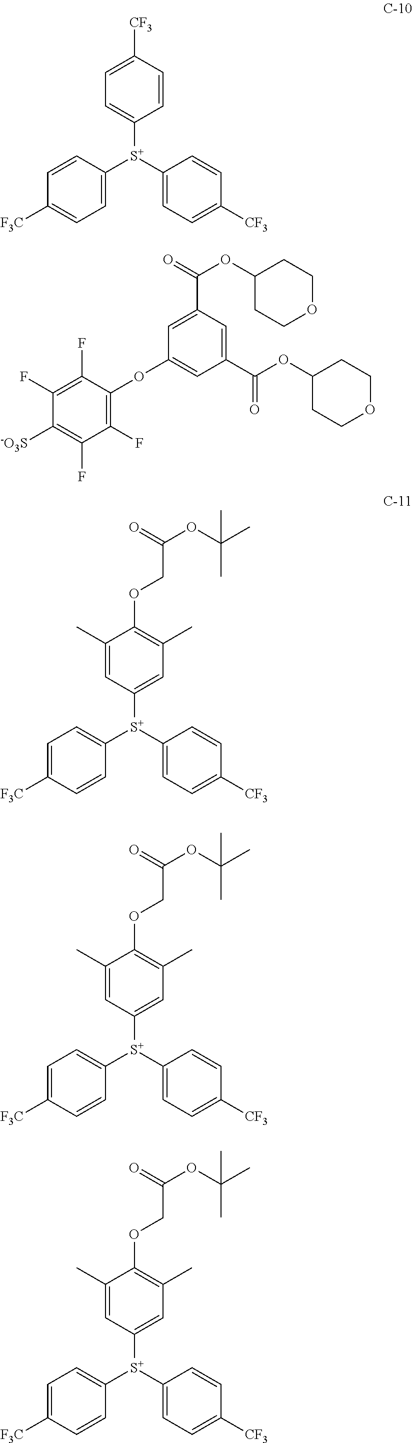

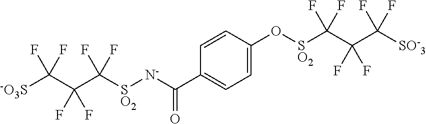

[Acid Diffusion Control Agent]

Structures of acid diffusion control agents (compounds D-1 to D-12) shown in Table 3 are shown below.

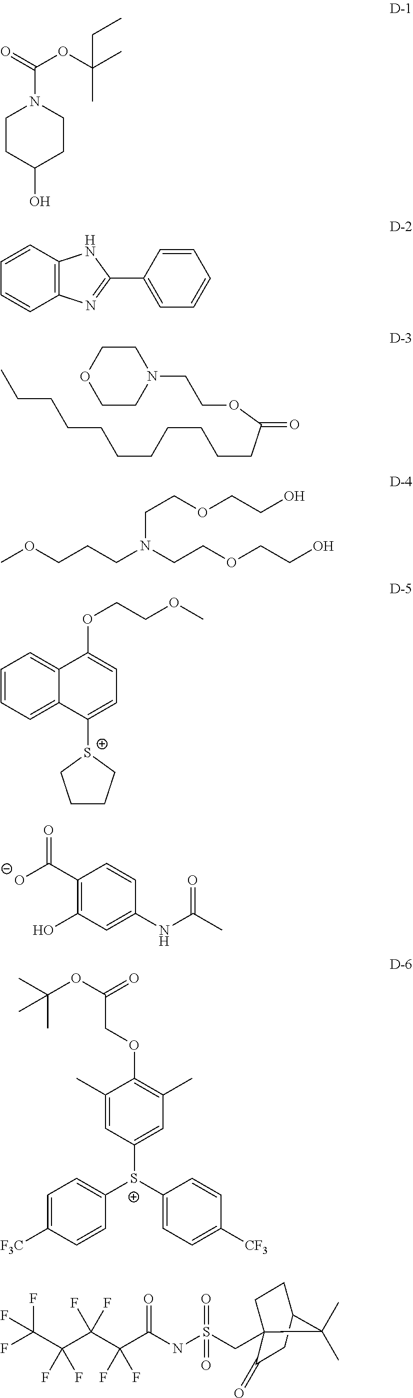

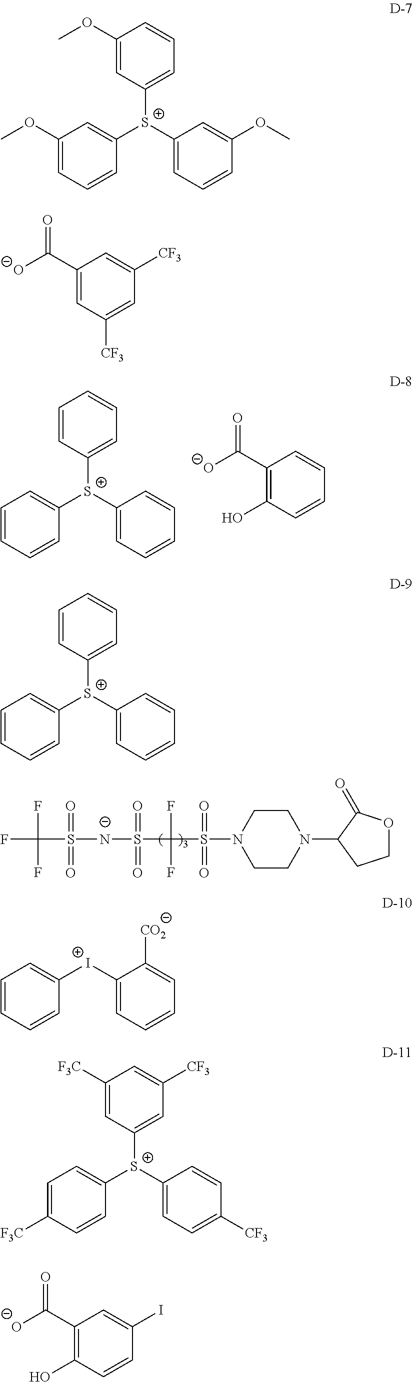

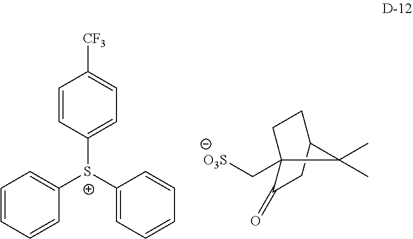

[Hydrophobic Resin]

Hydrophobic resins (resins E-1 to E-12) shown in Table 3 are shown below.

As the resins E-1 to E-12, those synthesized according to the method for synthesizing the resin A-1 (Synthesis Example 1) described above were used. A compositional ratio (ratio in % by mole) of each repeating unit shown below, a weight-average molecular weight (Mw), and a dispersity (Mw/Mn) are shown in Table 2.

The weight-average molecular weight (Mw) and the dispersity (Mw/Mn) of the resins E-1 to E-12 were measured by GPC (solvent carrier: tetrahydrofuran (Tf)) (an amount expressed in terms of polystyrene). In addition, the compositional ratio (ratio in % by mole) of the resin was measured by means of ¹³C-nuclear magnetic resonance (NMR).

TABLE 2

| | Molar ratio of | | Molar ratio of | | Molar ratio of | | Molar ratio of | | | |
|---|---|---|---|---|---|---|---|---|---|---|
| | repeating unit 1 | | repeating unit 2 | | repeating unit 3 | | repeating unit 4 | | Mw | Mw/Mn |
| Resin E-1 | ME-3 | 60 | ME-4 | 40 | | | | | 10000 | 1.40 |
| Resin E-2 | ME-15 | 50 | ME-1 | 50 | | | | | 12000 | 1.50 |
| Resin E-3 | ME-2 | 40 | ME-13 | 50 | ME-9 | 5 | ME-20 | 5 | 6000 | 1.30 |
| Resin E-4 | ME-19 | 50 | ME-14 | 50 | | | | | 9000 | 1.50 |
| Resin E-5 | ME-10 | 50 | ME-2 | 50 | | | | | 15000 | 1.50 |
| Resin E-6 | ME-17 | 50 | ME-15 | 50 | | | | | 10000 | 1.50 |
| Resin E-7 | ME-7 | 100 | | | | | | | 23000 | 1.70 |
| Resin E-8 | ME-5 | 100 | | | | | | | 13000 | 1.50 |
| Resin E-9 | ME-6 | 50 | ME-16 | 50 | | | | | 10000 | 1.70 |
| Resin E-10 | ME-13 | 10 | ME-18 | 85 | ME-12 | 5 | | | 11000 | 1.40 |
| Resin E-11 | ME-8 | 80 | ME-11 | 20 | | | | | 13000 | 1.40 |
| Resin E-12 | ME-15 | 50 | ME-21 | 50 | | | | | 6500 | 1.65 |

Structures of monomers ME-1 to VIE-21 corresponding to respective repeating units constituting resins the E-1 to E-12 shown in Table 2 are shown below.

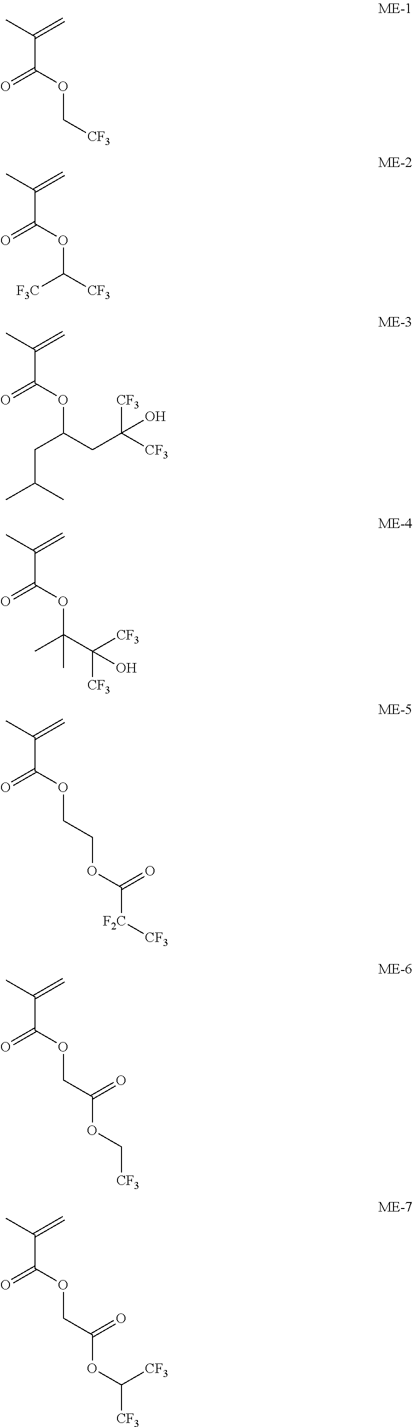

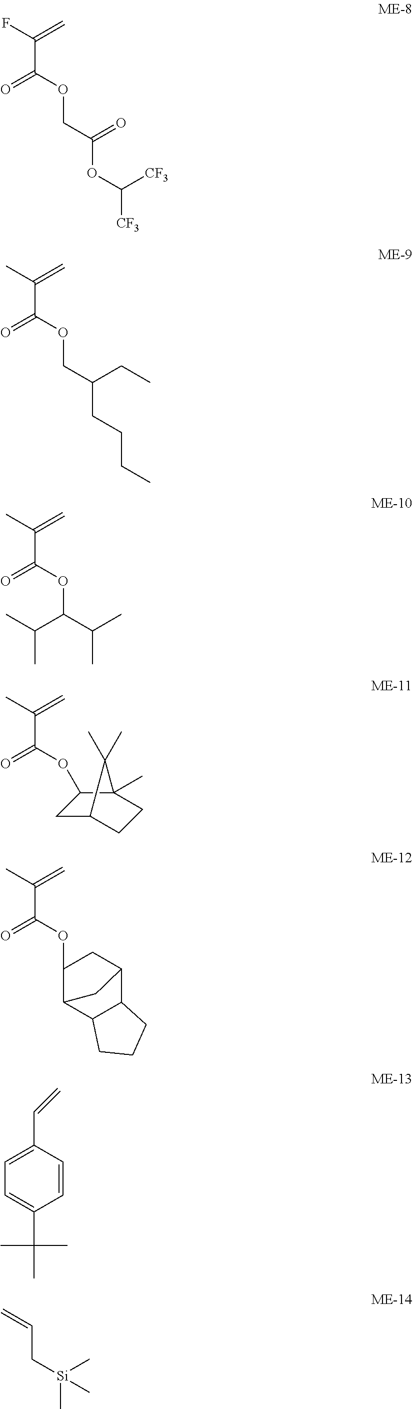

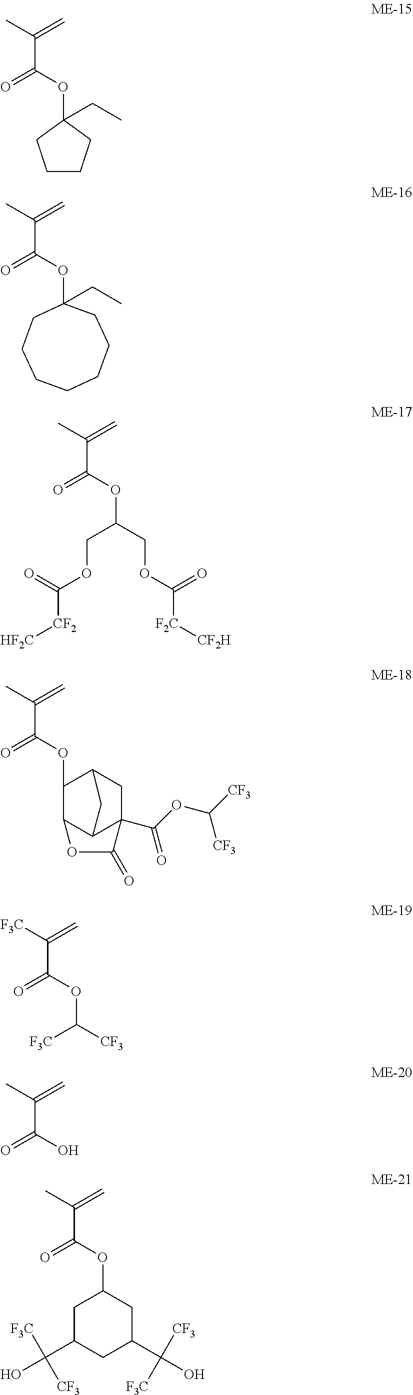

[Surfactant]

Surfactants shown in Table 3 are shown below.

H-1: MEGAFACE F176 (manufactured by DIC Corporation, fluorine-based surfactant)

H-2: MEGAFACE R08 (manufactured by DIC Corporation, fluorine- and silicon-based surfactant)

H-3: PF656 (manufactured by OMNOVA Solutions Inc., fluorine-based surfactant)

[Solvent]

Solvents shown in Table 3 are shown below.

F-1: Propylene glycol monomethyl ether acetate (PGMEA)

F-2: Propylene glycol monomethyl ether (PGME)

F-3: Propylene glycol monoethyl ether (PGEE)

F-4: Cyclohexanone

F-5: Cyclopentanone

F-6: 2-Heptanone

F-7: Ethyl lactate

F-8: -Butyrolactone

F-9: Propylene carbonate

F-10: Diacetone alcohol

### Examples 1 to 29 and Comparative Example 1

<Preparation of Resist Composition> (EB Exposure)

### Examples 1 to 20 and 23 to 29, Comparative Example 1

Components shown in Table 3 were dissolved in the solvent shown in Table 3 to prepare a solution having a concentration of solid contents of 2.3%, and the solution was filtered through a polyethylene filter having a pore size of 0.02 m to prepare a resist composition.

The solid content means all components excluding the solvent. The obtained resist compositions were used in Examples and Comparative Examples.

In addition, in the tables, the column of “Amount” indicates a content (% by mass) of each component with respect to the total solid content in the resist composition. In addition, the tables show the amount (part by mass) of the solvent used.

<Pattern Forming Method (1): EB Exposure and Alkali Development (Positive)>

### Examples 1 to 19 and 23 to 29, Comparative Example 1

The above-described resist composition was applied onto a 6-inch Si wafer pre-treated with hexamethyldisilazane (HAMIDS) using a spin coater Mark 8 manufactured by Tokyo Electron Limited, and dried on a hot plate at 100° C. for 60 seconds to obtain a resist film having a film thickness of 40 nm. Here, 1 inch is 0.0254 m.

In addition, even in a case where the Si wafer was changed to a chromium substrate, the same results could be obtained.

A wafer to which the resist film obtained above had been applied was subjected to patternwise irradiation using an electron beam drawing apparatus (HL750 manufactured by Hitachi, Ltd., accelerating voltage: 50 KeV). In this case, lithography was performed so that a 1:1 line-and-space was formed. After performing electron beam drawing, the wafer was heated on a hot plate at 100° C. for 60 seconds, developed with a 2.38% by mass tetramethylammonium hydroxide aqueous solution for 30 seconds, rinsed with pure water, rotated at a rotation speed of 4,000 rpm for 30 seconds, and then heated at 95° C. for 60 seconds to obtain a resist pattern with a 1:1 line-and-space pattern having a line width of 50 nm.

<Performance Evaluation>

[Resolution]

A cross-sectional shape of the obtained pattern was observed with a scanning electron microscope (5-4300 manufactured by Hitachi, Ltd.). An exposure amount (amount of electron beam irradiation) upon resolution of a 1:1 line-and-space resist pattern having a line width of 50 nm was defined as a sensitivity (Eop).

A critical resolving power (minimum line width at which lines and spaces (line:space=1:1) were separated and resolved) at the above-described exposure amount showing the sensitivity was defined as an L/S resolving power (nm).

<Pattern Forming Method (2): EB Exposure and Organic Solvent Development (Negative)>

### Example 20

The above-described resist composition was applied onto a 6-inch Si wafer pre-treated with hexamethyldisilazane (HMDS) using a spin coater Mark 8 manufactured by Tokyo Electron Limited, and dried on a hot plate at 100° C. for 60 seconds to obtain a resist film having a film thickness of 40 nm.

A wafer to which the resist film obtained above had been applied was subjected to patternwise irradiation using an electron beam drawing apparatus (HL750 manufactured by Hitachi, Ltd., accelerating voltage: 50 KeV). In this case, lithography was performed so that a 1:1 line-and-space was formed. After performing electron beam drawing, the wafer was heated on a hot plate at 100° C. for 60 seconds, developed with n-butyl acetate for 30 seconds, spin-dried, and then heated at 95° C. for 60 seconds to obtain a resist pattern with a 1:1 line-and-space pattern having a line width of 50 nm.

<Performance Evaluation>

[Resolution]

A cross-sectional shape of the obtained pattern was observed with a scanning electron microscope (S-4300 manufactured by Hitachi, Ltd.). An exposure amount (amount of electron beam irradiation) upon resolution of a 1:1 line-and-space resist pattern having a line width of 50 nm was defined as a sensitivity (Eop).

A critical resolving power (minimum line width at which lines and spaces (line:space=1:1) were separated and resolved) at the above-described exposure amount showing the sensitivity was defined as an L/S resolving power (nm).

<Preparation of Resist Composition>(EUV Exposure)

### Examples 21 and 22

Components shown in Table 3 were dissolved in the solvent shown in Table 3 to prepare a solution having a concentration of solid contents of 2.3%, and the solution was filtered through a polyethylene filter having a pore size of 0.02 m to prepare a resist composition.

The solid content means all components excluding the solvent. The obtained resist compositions were used in Examples and Comparative Examples.

In addition, in the tables, the column of “Amount” indicates a content (% by mass) of each component with respect to the total solid content in the resist composition. In addition, the tables show the amount (part by mass) of the solvent used.

<Pattern Forming Method (3): EUV Exposure and Alkali Development (Positive)>

### Example 21

A composition for forming an underlayer film, AL412 (manufactured by Brewer Science, Inc.), was applied to a silicon wafer and baked at 205° C. for 60 seconds to form an underlying film having a film thickness of 20 nm. A resist composition shown in Table was applied thereto and baked at 100° C. for 60 seconds to form a resist film having a film thickness of 40 nm.

The silicon wafer having the obtained resist film was subjected to a pattern irradiation using an EUV exposure device (manufactured by Exitech Ltd., Micro Exposure Tool, NA 0.3, Quadrupole, outer sigma 0.68, inner sigma 0.36). As a reticle, a mask having a line size=20 nm and a line:space=1:1 was used.

The resist film after the exposure was baked at 90° C. for 60 seconds, developed with a tetramethylammonium hydroxide aqueous solution (2.38% by mass) for 30 seconds, and then rinsed with pure water for 30 seconds. Thereafter, the resist film was spin-dried to obtain a positive tone pattern.

<Performance Evaluation>

[Resolution]

A cross-sectional shape of the obtained pattern was observed with a scanning electron microscope (S-4300 manufactured by Hitachi, Ltd.). An exposure amount (amount of electron beam irradiation) upon resolution of a 1:1 line-and-space resist pattern having a line width of 20 nm was defined as a sensitivity (Eop).

A critical resolving power (minimum line width at which lines and spaces (line:space=1:1) were separated and resolved) at the above-described exposure amount showing the sensitivity was defined as an L/S resolving power (nm).

<Pattern Forming Method (4): EUV Exposure and Organic Solvent Development (Negative)>

### Example 22

A composition for forming an underlayer film, AL412 (manufactured by Brewer Science, Inc.), was applied to a silicon wafer and baked at 205° C. for 60 seconds to form an underlying film having a film thickness of 20 nm. A resist composition shown in Table was applied thereto and baked at 100° C. for 60 seconds to form a resist film having a film thickness of 40 nm.

The silicon wafer having the obtained resist film was subjected to a pattern irradiation using an EUV exposure device (manufactured by Exitech Ltd., Micro Exposure Tool, NA 0.3, Quadrupole, outer sigma 0.68, inner sigma 0.36). As a reticle, a mask having a line size=20 nm and a line:space=1:1 was used.

The resist film after the exposure was baked at 90° C. for 60 seconds, developed with n-butyl acetate for 30 seconds, and spin-dried to obtain a negative tone pattern.

<Performance Evaluation>

[Resolution]

A cross-sectional shape of the obtained pattern was observed with a scanning electron microscope (S-4300 manufactured by Hitachi, Ltd.). An exposure amount (amount of electron beam irradiation) upon resolution of a 1:1 line-and-space resist pattern having a line width of 20 nm was defined as a sensitivity (Eop).

A critical resolving power (minimum line width at which lines and spaces (line:space=1:1) were separated and resolved) at the above-described exposure amount showing the sensitivity was defined as an L/S resolving power (nm).

The obtained evaluation results are shown in Table 4.

TABLE 3

| | | Resin | | Photoacid generator | | Photoacid generator | | Acid diffusion | |
|---|---|---|---|---|---|---|---|---|---|
| | Resist | (A) | | (B) | | (C) | | control agent | |
| | composition | Type | Amount | Type | Amount | Type | Amount | Type | Amount |
| Example 1 | Re-1 | A-1 | 70.0 | B-4 | 30.0 | — | — | — | — |
| Example 2 | Re-2 | A-2 | 70.0 | B-7 | 14.0 | C-5 | 10.0 | D-7 | 3.0 |
| Example 3 | Re-3 | A-3 | 55.0 | B-3 | 38.0 | — | — | D-2 | 2.0 |
| Example 4 | Re-4 | A-4 | 78.0 | B-2 | 17.0 | — | — | D-11 | 5.0 |
| Example 5 | Re-5 | A-5 | 55.0 | B-5 | 35.0 | C-4 | 3.0 | D-1 | 5.0 |
| Example 6 | Re-6 | A-6 | 37.9 | B-4 | 60.0 | — | — | D-8 | 2.0 |
| Example 7 | Re-7 | A-7 | 64.0 | B-6 | 36.0 | — | — | — | — |
| Example 8 | Re-8 | A-8 | 89.5 | B-1 | 5.0 | — | — | D-1 | 4.0 |
| Example 9 | Re-9 | A-9 | 50.5 | B-6 | 45.0 | C-1 | 2.5 | — | |
| Example 10 | Re-10 | A-10 | 70.0 | B-4 | 30.0 | — | — | — | — |
| Example 11 | Re-11 | A-11 | 70.8 | B-3 | 26.0 | — | — | D-3 | 2.0 |
| Example 12 | Re-12 | A-12 | 67.0 | B-2 | 16.0 | C-2 | 12.0 | D-4 | 5.0 |
| Example 13 | Re-13 | A-13 | 71.5 | B-7 | 22.0 | — | — | D-10 | 3.5 |
| Example 14 | Re-14 | A-14 | 65.0 | B-1 | 34.0 | — | — | D-12 | 1.0 |
| Example 15 | Re-15 | A-15 | 79.9 | B-2 | 15.0 | C-6 | 1.5 | D-4 | 2.5 |
| Example 16 | Re-16 | A-16 | 83.0 | B-3 | 17.0 | — | — | — | — |
| Example 17 | Re-17 | A-17 | 86.5 | B-5 | 7.0 | C-3 | 5.5 | — | — |
| Example 18 | Re-18 | A-18 | 64.0 | B-8 | 32.0 | — | — | D-12 | 4.0 |
| Example 19 | Re-19 | A-19 | 89.9 | B-6 | 4.0 | — | — | D-5 | 3.0 |
| Example 20 | Re-20 | A-20 | 61.0 | B-5 | 34.0 | — | — | D-9 | 3.0 |
| Example 21 | Re-21 | A-21 | 73.0 | B-1 | 20.0 | C-7 | 4.0 | D-6 | 3.0 |
| Example 22 | Re-22 | A-22 | 83.5 | B-7 | 15.0 | — | — | — | — |
| Example 23 | Re-23 | A-23 | 66.5 | B-8 | 28.0 | C-3 | 5.5 | — | — |
| Example 24 | Re-24 | A-24 | 63.9 | B-2 | 36.0 | — | — | — | — |
| Example 25 | Re-25 | A-25 | 64.0 | B-1 | 36.0 | — | — | — | |
| Example 26 | Re-26 | A-7 | 60.0 | B-6 | 36.0 | C-11 | 4.0 | — | — |
| Example 27 | Re-27 | A-18 | 60.5 | B-10 | 32.0 | C-8 | 3.5 | D-12 | 4.0 |
| Example 28 | Re-28 | A-23 | 66.5 | B-11 | 28.0 | C-9 | 5.5 | — | |
| Example 29 | Re-29 | A-1 | 67.0 | B-12 | 30.0 | C-10 | 3.0 | — | |
| Comparative Example 1 | X-1 | A-1 | 70.0 | B-9 | 30.0 | — | — | — | — |

| | Hydrophobic resin | | Surfactant | | Solvent | | Pattern forming |
|---|---|---|---|---|---|---|---|
| | Type | Amount | Type | Amount | Type | Mixed ratio | method |
| Example 1 | — | — | — | — | F-1/F-2 | 80/20 | EB-alkali |
| Example 2 | E-4 | 3.0 | — | — | F-1/F-2/F-8 | 50/40/10 | EB-alkali |
| Example 3 | E-1 | 5.0 | — | — | F-1/F-2/F-8 | 70/25/5 | EB-alkali |
| Example 4 | — | — | — | — | F-1/F-3 | 70/30 | EB-alkali |
| Example 5 | E-5 | 2.0 | — | — | F-1/F-2 | 70/30 | EB-alkali |
| Example 6 | — | — | H-1 | 0.1 | F-1/F-8 | 85/15 | EB-alkali |
| Example 7 | — | — | — | — | F-1/F-10 | 50/50 | EB-alkali |
| Example 8 | E-8 | 1.5 | — | — | F-1/F-8 | 85/15 | EB-alkali |
| Example 9 | E-11 | 2.0 | — | — | F-1 | 100 | EB-alkali |
| Example 10 | — | — | — | — | F-1/F-9 | 90/10 | EB-alkali |
| Example 11 | E-2 | 1.2 | — | — | F-1/F-7 | 90/10 | EB-alkali |
| Example 12 | — | — | — | — | F-1/F-6 | 40/60 | EB-alkali |
| Example 13 | E-6 | 3.0 | — | — | F-1/F-8 | 85/15 | EB-alkali |
| Example 14 | — | — | — | — | F-1/F-8 | 85/15 | EB-alkali |
| Example 15 | E-7 | 1.0 | H-2 | 0.1 | F-1/F-8 | 85/15 | EB-alkali |
| Example 16 | — | — | — | — | F-4 | 100 | EB-alkali |
| Example 17 | E-10 | 1.0 | — | — | F-1/F-10 | 85/15 | EB-alkali |
| Example 18 | — | — | — | — | F-1/F-8 | 85/15 | EB-alkali |
| Example 19 | E-3 | 3.0 | H-3 | 0.1 | F-1/F-8 | 85/15 | EB-alkali |
| Example 20 | E-12 | 2.0 | — | — | F-1/F-5 | 70/30 | EB-organic solvent |
| Example 21 | — | — | — | — | F-9 | 100 | EUV-alkali |
| Example 22 | E-9 | 1.5 | — | — | F-1/F-4 | 70/30 | EUV-organic solvent |
| Example 23 | — | — | — | — | F-1/F-10 | 50/50 | EB-alkali |
| Example 24 | — | — | H-1 | 0.1 | F-1/F-10 | 50/50 | EB-alkali |
| Example 25 | — | — | — | — | F-1/F-10 | 50/50 | EB-alkali |
| Example 26 | — | — | — | — | F-1/F-10 | 50/50 | EB-alkali |
| Example 27 | — | | — | — | F-1/F-8 | 85/15 | EB-alkali |
| Example 28 | — | — | — | — | F-1/F-10 | 50/50 | EB-alkali |
| Example 29 | — | | — | — | F-1/F-2 | 80/20 | EB-alkali |
| Comparative Example 1 | — | — | — | — | F-1/F-2 | 80/20 | EB-alkali |

TABLE 4

| | Pattern | L/S resolving |
|---|---|---|
| | forming method | power (nm) |
| Example 1 | EB-alkali | 19 |
| Example 2 | EB-alkali | 18 |
| Example 3 | EB-alkali | 17 |
| Example 4 | EB-alkali | 18 |
| Example 5 | EB-alkali | 17 |
| Example 6 | EB-alkali | 17 |
| Example 7 | EB-alkali | 16 |
| Example 8 | EB-alkali | 18 |
| Example 9 | EB-alkali | 17 |
| Example 10 | EB-alkali | 18 |
| Example 11 | EB-alkali | 19 |
| Example 12 | EB-alkali | 18 |
| Example 13 | EB-alkali | 17 |
| Example 14 | EB-alkali | 18 |
| Example 15 | EB-alkali | 17 |
| Example 16 | EB-alkali | 19 |
| Example 17 | EB-alkali | 20 |
| Example 18 | EB-alkali | 16 |
| Example 19 | EB-alkali | 15 |
| Example 20 | EB-organic solvent | 18 |
| Example 21 | EUV-alkali | 19 |
| Example 22 | EUV-organic solvent | 15 |
| Example 23 | EB-alkali | 16 |
| Example 24 | EB-alkali | 17 |
| Example 25 | EB-alkali | 18 |
| Example 26 | EB-alkali | 16 |
| Example 27 | EB-alkali | 18 |
| Example 28 | EB-alkali | 17 |
| Example 29 | EB-alkali | 18 |
| Comparative | EB-alkali | 28 |
| Example 1 | | |

As shown in Table 4 above, it was confirmed that the resist composition according to the embodiment of the present invention had excellent resolution in a case where an extremely fine pattern of 20 nm or less was formed by the alkali development or the organic solvent development. On the other hand, the resist compositions of Comparative Examples were insufficient in the performance.

According to the present invention, it is possible to provide an actinic ray-sensitive or radiation-sensitive resin composition in which, in a formation of an extremely fine pattern (for example, line-and-space pattern having a line width or space width of 20 nm or less, hole pattern of a pore diameter of 20 nm or less, and the like), a resolution is extremely excellent.

In addition, according to the present invention, it is possible to provide an actinic ray-sensitive or radiation-sensitive film formed of the actinic ray-sensitive or radiation-sensitive resin composition, a pattern forming method, and a method for manufacturing an electronic device.

The present invention has been described in detail with reference to specific embodiments. To those skilled in the art, it is obvious that various changes or modifications can be added without departing from the gist and scope of the present invention.

The present application is based on Japanese Patent Application (JP2021-47795) filed on Mar. 22, 2021 and Japanese Patent Application (JP2021-126332) filed on Jul. 30, 2021, and the contents of which are incorporated herein by reference.

What is claimed is:

1. An actinic ray-sensitive or radiation-sensitive resin composition comprising:
(A) a resin which is decomposed by action of acid to increase polarity; and
(B) a compound which generates an acid by irradiation with an actinic ray or a radiation,
wherein the resin (A) and the acid generated from the compound (B) form a bond by the actinic ray or the radiation or by the action of acid.

2. The actinic ray-sensitive or radiation-sensitive resin composition according to claim 1,
wherein the resin (A) is a resin having a reactive moiety (1),
the compound (B) is an ionic compound having a reactive moiety (2) in an anionic moiety, and
a reactive species generated from one of the reactive moiety (1) or the reactive moiety (2) by the actinic ray or the radiation or by the action of acid reacts with the other of the reactive moiety (1) and the reactive moiety (2) to form the bond.

3. The actinic ray-sensitive or radiation-sensitive resin composition according to claim 2,
wherein the compound (B) is an ionic compound having, as the reactive moiety (2) in the anionic moiety, a partial structure represented by any of General Formula (1), (2), or (3), 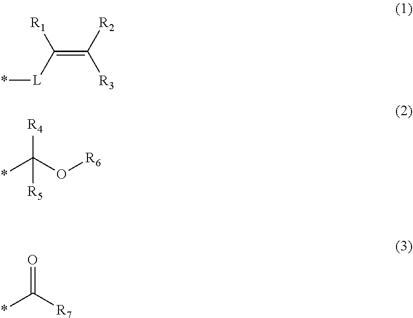
in the General Formula (1), R₁ to R₃ each independently represent a hydrogen atom or a substituent, L represents a single bond or a divalent linking group, and * represents a bonding position,
in the General Formula (2), R₄ to R₆ each independently represent a hydrogen atom or a substituent, and * represents a bonding position,
in the General Formula (3), R₇ represents a hydrogen atom or a substituent, and * represents a bonding position.

4. The actinic ray-sensitive or radiation-sensitive resin composition according to claim 3,
wherein the partial structure is the partial structure represented by the General Formula (1) or the General Formula (3).

5. The actinic ray-sensitive or radiation-sensitive resin composition according to claim 3,
wherein the partial structure is a partial structure selected from the following structures, 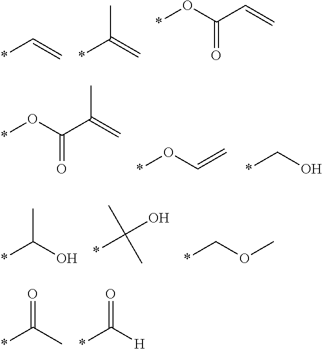
* represents a bonding position.

6. The actinic ray-sensitive or radiation-sensitive resin composition according to claim 5,
wherein the partial structure is a partial structure selected from the following structures, 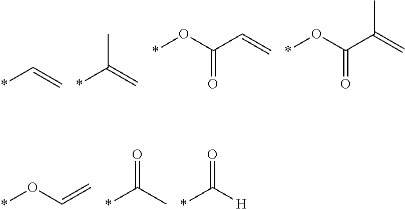
* represents a bonding position.

7. An actinic ray-sensitive or radiation-sensitive resin composition comprising:
(A) a resin which is decomposed by action of acid to increase polarity; and
(B) a compound which generates an acid by irradiation with an actinic ray or a radiation,
wherein the resin (A) is a resin having an acid group, an alcoholic hydroxyl group, or an acid-decomposable group, and
the compound (B) is an ionic compound having a partial structure represented by any of General Formula (1), (2), or (3) in an anionic moiety, 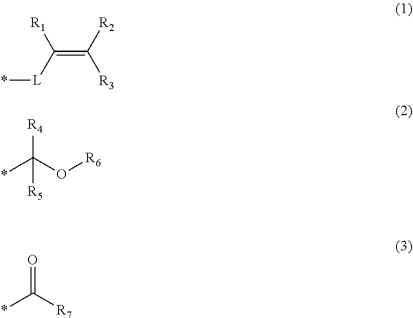
in the General Formula (1), R₁ to R₃ each independently represent a hydrogen atom or a substituent, L represents a single bond or a divalent linking group, and * represents a bonding position,
in the General Formula (2), R₄ to R₅ each independently represent a hydrogen atom or a substituent, and * represents a bonding position,
in the General Formula (3), R₇ represents a hydrogen atom or a substituent, and * represents a bonding position.

8. The actinic ray-sensitive or radiation-sensitive resin composition according to claim 7,
wherein the partial structure is the partial structure represented by the General Formula (1) or the General Formula (3).

9. The actinic ray-sensitive or radiation-sensitive resin composition according to claim 7,
wherein the partial structure is a partial structure selected from the following structures, 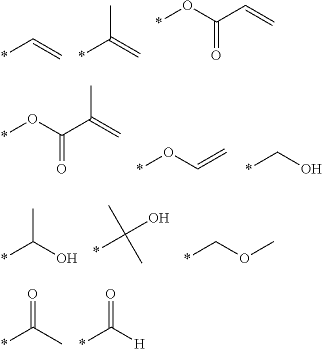
* represents a bonding position.

10. The actinic ray-sensitive or radiation-sensitive resin composition according to claim 9,
wherein the partial structure is a partial structure selected from the following structures, 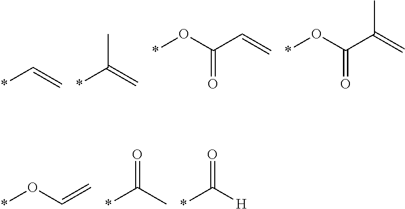
* represents a bonding position.

11. The actinic ray-sensitive or radiation-sensitive resin composition according to claim 2,
wherein the compound (B) has no partial structure represented by any of General Formula (11), (12), (13), or (14) in the anionic moiety, 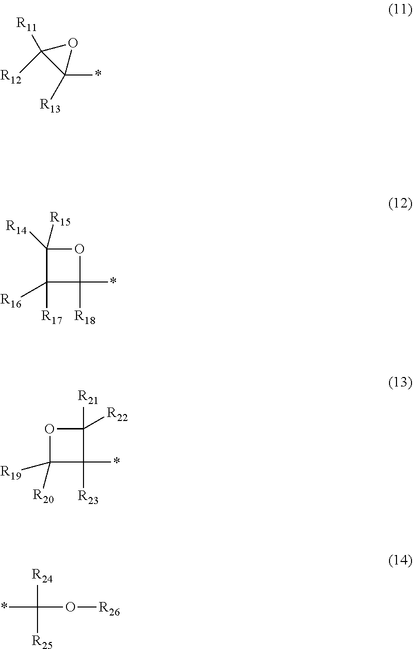
in the General Formula (11), Rₙ to R₁₃ each independently represent a hydrogen atom or a substituent, and * represents a bonding position,
in the General Formula (12), R₁₄ to R₁₅ each independently represent a hydrogen atom or a substituent, and * represents a bonding position,
in the General Formula (13), R₁₉ to R₂₃ each independently represent a hydrogen atom or a substituent, and * represents a bonding position,
in the General Formula (14), R₂₄ to R₂₆ each independently represent a hydrogen atom or a substituent, and * represents a bonding position.

12. The actinic ray-sensitive or radiation-sensitive resin composition according to claim 1,
wherein the resin (A) has a dissociable hydrogen atom.

13. The actinic ray-sensitive or radiation-sensitive resin composition according to claim 1,
wherein the resin (A) has a phenolic hydroxyl group.

14. The actinic ray-sensitive or radiation-sensitive resin composition according to claim 1,
wherein the acid generated from the compound (B) includes an aromatic ring.

15. An actinic ray-sensitive or radiation-sensitive film formed of the actinic ray-sensitive or radiation-sensitive resin composition according to claim 1.

16. A pattern forming method comprising:
forming an actinic ray-sensitive or radiation-sensitive film on a substrate using the actinic ray-sensitive or radiation-sensitive resin composition according to claim 1;
exposing the actinic ray-sensitive or radiation-sensitive film; and
developing the exposed actinic ray-sensitive or radiation-sensitive film with a developer to form a pattern.

17. A method for manufacturing an electronic device, comprising:
the pattern forming method according to claim 16.
